SEQUENCE LISTING (71 sequences)

SEQ ID NO: 1 - DNA, 4308 bp, Streptococcus sobrinus
  source 1..4308: mol_type = unassigned DNA
atggttgacggcaaatactactattatgatcaggacggtaacgtaaagaagaatttcgcggtgagcgttggtgacaaaatctactacttcgatgaaactggtgcatataaggataccagcaaagtggacgccgacaagagcagcagcgcggttagccaaaacgcgaccatctttgcggcgaataaccgtgcgtacagcacctctgcaaagaattttgaagcggtggataactacctgaccgcagacagctggtatcgtccgaaatccatcctgaaggacggcaaaacctggaccgagagcggtaaggatgatttccgtccactgctgatggcatggtggcctgacaccgaaactaagcgcaactacgtgaactatatgaataaagtggtcggtattgacaagacgtacactgcggaaacgtcgcaagcggatttgaccgcagcggcggagctggttcaagcgcgtatcgagcagaagattaccagcgaaaacaacaccaaatggctgcgcgaagcaatctccgcgttcgttaagacgcagcctcagtggaacggcgagtccgaaaagccgtatgacgatcacttgcagaacggtgcgctgctgtttgataaccaaaccgacctgacgccagacacccaaagcaattaccgtttgctgaaccgtaccccgaccaatcagactggtagcctggatagccgttttacgtataatccgaatgacccgttgggcggctacgatttcttgctggcgaacgacgttgacaatagcaatccggtcgtccaggctgaacagttgaactggctgcattatctgctgaactttggctctatttacgctaacgatgccgacgccaattttgacagcattcgcgttgatgccgtcgataatgtcgatgctgatctgctgcaaatcagcagcgattacctgaaagcagcgtatggcatcgacaagaataacaagaatgcgaacaaccatgttagcatcgtcgaagcgtggagcgacaatgataccccgtatttgcacgacgatggcgataatctgatgaacatggacaacaaatttcgcctgtccatgctgtggagcctggcaaagccgctggacaaacgtagcggtttgaacccgctgattcacaatagcctggtggaccgcgaggtggacgatcgtgaagtggaaaccgtgccgtcctacagctttgctcgtgcacatgatagcgaggtgcaggacatcatccgtgacattatcaaggctgagattaacccaaatagctttggttatagcttcactcaagaagagatcgagcaagcctttaagatttacaacgaggatttgaagaaaacggacaagaaatacacccactacaatgtgccgctgagctacaccctgctgctgaccaacaagggcagcatcccgcgtgtgtactatggtgatatgttcaccgatgatggccaatacatggcaaacaagaccgtcaactacgacgcaatcgagagcctgctgaaagcccgtatgaaatatgtcagcggtggccaagcaatgcagaactatcaaattggtaatggcgagattttgaccagcgtgcgctatggtaaaggtgccctgaagcagagcgataagggtgacgcgacgacgcgcactagcggtgttggcgtggttatgggtaatcagccgaacttctccctggacggtaaagttgtggccctgaatatgggtgcggcccatgcgaatcaagaataccgtgcactgatggtcagcactaaagacggtgtggcaacttacgcaaccgatgctgacgcatccaaagcgggcctggtcaagcgtaccgacgagaacggctacctgtacttcctgaatgatgatctgaagggcgtcgcgaaccctcaggtttccggcttcttgcaagtgtgggttccagttggtgccgccgatgaccaggacattcgcgtcgccgccagcgacacggcgagcacggatggtaaaagcctgcatcaagatgcggcgatggacagccgcgtcatgtttgagggtttcagcaattttcaatccttcgcgaccaaagaagaagaatacacgaatgttgttatcgcgaacaatgtcgataagttcgttagctggggtatcaccgattttgaaatggctccgcagtatgttagcagcaccgacggtcagttcttggacagcgtcatccagaatggctatgcgtttactgatcgctatgatctgggtatgtccaaggcgaacaagtatggcacggcagaccaactggttaaggcaatcaaagccctgcacgctaaaggcctgaaagttatggcggactgggtcccggatcaaatgtacacctttccaaaacaggaagttgtgaccgttacccgcaccgacaaattcggtaaaccgatcgccggctctcaaatcaatcacagcttgtatgtgaccgacaccaaatccagcggcgacgactaccaagcgaagtacggcggtgccttcctggatgaactgaaagaaaagtacccggaactgttcacgaaaaagcaaattagcacgggccaagcgattgatccgagcgtgaaaatcaagcagtggagcgcaaaatacttcaatggttcgaatatcctgggtcgcggtgcggactatgtgctgagcgaccaggtcagcaataagtatttcaacgtggcgagcgacaccttgttcctgccgtccagcctgctgggcaaggtcgtggagagcggcattcgttacgacggcaagggttacatctacaacagctccgcgaccggcgatcaggtcaaagcgtctttcattacggaagccggtaacctgtattacttcggcaaagacggttacatggttactggtgcccagacgattaatggcgccaactacttcttcctggaaaacggtacggcactgcgtaatacgatttacaccgatgctcaaggtaatagccactattacgcgaatgatggcaaacgctatgaaaatggctatcaacagttcggtaacgattggcgctactttaaagatggtaacatggcagtcggcctgaccacggttgatggcaacgtgcaatactttgacaaagacggcgtccaggcaaaggataagattatcgtcacccgtgatggcaaggtccgttacttcgatcagcacaacggtaacgcggcgaccaacacgttcattgctgataaaactggccattggtattacctgggtaaagatggcgtcgcggtgactggcgcccagaccgtcggcaaacaaaaactgtacttcgaggccaacggtcaacaagttaaaggtgactttgttacgtccgatgagggcaaactgtatttctatgacgttgattctggtgacatgtggacggacaccttcatcgaggataaggcgggcaactggttctatttgggcaaggatggtgcggcagttacgggtgcccaaacgattcgcggtcagaagctgtacttcaaggccaatggtcaacaggtcaagggtgacattgttaagggcaccgacggtaaaatccgctactatgatgcaaaatccggtgaacaggtgttcaacaaaacggtgaaagctgcggatggcaaaacgtatgttatcggtaatgatggtgtcgcggtggaccctagcgtggttaaaggtcaaacctttaaggacgcttcgggcgctctgcgtttctacaacttgaagggtcaactggtcactggcagcggctggtatgaaaccgcgaaccatgactgggtttacattcagtccggcaaggcactgaccggcgaacagaccattaacggtcaacacctgtatttcaaagaagatggtcaccaagtcaagggtcagttggtcacgggcaccgatggtaaagtgcgttactatgacgccaacagcggtgaccaagcattcaacaagagcgtcactgtgaatggtaaaacctattactttggcaacgatggtacggcgcagactgctggcaacccgaagggtcagacgttcaaggatggctccgacatccgtttttactctatggaaggccaactggtgaccggctcgggttggtacgagaacgcgcaaggccagtggctgtatgtgaaaaacggtaaggtgctgactggtctgcaaaccgttggcagccagcgtgtttacttcgacgagaatggtattcaggccaagggcaaagcagtgcgtaccagcgatggcaaaattcgttatttcgacgaaaacagcggcagcatgatcacgaatcaatggaagttcgtctatggtcagtattactactttggtaacgacggtgcacgtatttaccgtggttggaactaa

SEQ ID NO: 2 - protein, 1435 aa, Streptococcus sobrinus
MVDGKYYYYDQDGNVKKNFAVSVGDKIYYFDETGAYKDTSKVDADKSSSAVSQNATIFAANNRAYSTSAKNFEAVDNYLTADSWYRPKSILKDGKTWTESGKDDFRPLLMAWWPDTETKRNYVNYMNKVVGIDKTYTAETSQADLTAAAELVQARIEQKITSENNTKWLREAISAFVKTQPQWNGESEKPYDDHLQNGALLFDNQTDLTPDTQSNYRLLNRTPTNQTGSLDSRFTYNPNDPLGGYDFLLANDVDNSNPVVQAEQLNWLHYLLNFGSIYANDADANFDSIRVDAVDNVDADLLQISSDYLKAAYGIDKNNKNANNHVSIVEAWSDNDTPYLHDDGDNLMNMDNKFRLSMLWSLAKPLDKRSGLNPLIHNSLVDREVDDREVETVPSYSFARAHDSEVQDIIRDIIKAEINPNSFGYSFTQEEIEQAFKIYNEDLKKTDKKYTHYNVPLSYTLLLTNKGSIPRVYYGDMFTDDGQYMANKTVNYDAIESLLKARMKYVSGGQAMQNYQIGNGEILTSVRYGKGALKQSDKGDATTRTSGVGVVMGNQPNFSLDGKVVALNMGAAHANQEYRALMVSTKDGVATYATDADASKAGLVKRTDENGYLYFLNDDLKGVANPQVSGFLQVWVPVGAADDQDIRVAASDTASTDGKSLHQDAAMDSRVMFEGFSNFQSFATKEEEYTNVVIANNVDKFVSWGITDFEMAPQYVSSTDGQFLDSVIQNGYAFTDRYDLGMSKANKYGTADQLVKAIKALHAKGLKVMADWVPDQMYTFPKQEVVTVTRTDKFGKPIAGSQINHSLYVTDTKSSGDDYQAKYGGAFLDELKEKYPELFTKKQISTGQAIDPSVKIKQWSAKYFNGSNILGRGADYVLSDQVSNKYFNVASDTLFLPSSLLGKVVESGIRYDGKGYIYNSSATGDQVKASFITEAGNLYYFGKDGYMVTGAQTINGANYFFLENGTALRNTIYTDAQGNSHYYANDGKRYENGYQQFGNDWRYFKDGNMAVGLTTVDGNVQYFDKDGVQAKDKIIVTRDGKVRYFDQHNGNAATNTFIADKTGHWYYLGKDGVAVTGAQTVGKQKLYFEANGQQVKGDFVTSDEGKLYFYDVDSGDMWTDTFIEDKAGNWFYLGKDGAAVTGAQTIRGQKLYFKANGQQVKGDIVKGTDGKIRYYDAKSGEQVFNKTVKAADGKTYVIGNDGVAVDPSVVKGQTFKDASGALRFYNLKGQLVTGSGWYETANHDWVYIQSGKALTGEQTINGQHLYFKEDGHQVKGQLVTGTDGKVRYYDANSGDQAFNKSVTVNGKTYYFGNDGTAQTAGNPKGQTFKDGSDIRFYSMEGQLVTGSGWYENAQGQWLYVKNGKVLTGLQTVGSQRVYFDENGIQAKGKAVRTSDGKIRYFDENSGSMITNQWKFVYGQYYYFGNDGARIYRGWN

SEQ ID NO: 3 - DNA, 4026 bp, Streptococcus salivarius
  source 1..4026: mol_type = other DNA
atgatcgacggcaaatactattatgttaatgaggacggtagccacaaagaaaactttgcgattacggttaatggtcaactgctgtatttcggtaaggacggcgcactgacctctagcagcacttacagctttaccccaggtacgacgaacatcgtggatggcttttctatcaacaaccgcgcgtatgactccagcgaagcgtcctttgaactgattgatggctacttgactgccgactcctggtatcgtccggcttccatcatcaaggacggtgtcacgtggcaggccagcaccgcagaggactttcgcccgctgctgatggcgtggtggccaaacgtggatacccaggtgaactatctgaactacatgtctaaagtgtttaacctggacgcaaagtatagcagcaccgataaacaagagactctgaaggttgcagctaaggatattcagattaagatcgagcagaaaattcaggcggagaaaagcacccaatggctgcgcgaaacgatcagcgcttttgtgaaaacccaaccacagtggaacaaagagactgagaattactcgaaaggtggtggtgaggatcatctgcaaggcggtgcactgctgtacgtgaatgatagccgtaccccgtgggcaaatagcgattatcgccgcctgaaccgcaccgctaccaatcaaacgggtacgattgacaagtccattctggacgagcagagcgacccaaatcacatgggcggtttcgacttcctgctggcgaatgatgttgacctgtccaacccggttgtgcaggcagagcagctgaaccagattcactacttgatgaattggggctctatcgtgatgggtgacaaagacgcaaactttgatggtatccgtgtcgatgcagttgacaacgtcgatgccgacatgctgcaactgtataccaactacttccgtgaatactacggtgttaacaaaagcgaagcgaacgcactggcgcacattagcgttttggaagcgtggagcttgaatgataatcactacaacgacaaaaccgatggtgcagcattggcgatggagaataagcagcgtctggcgctgctgtttagcctggctaaaccgattaaagagcgcaccccggcagtgagcccgctgtataacaacaccttcaatacgacccaacgcgatgagaaaaccgactggatcaataaagacggttctaaggcctataacgaggatggtactgtgaagcagagcaccattggtaagtacaatgaaaaatatggtgatgcatcgggcaattatgtgttcatccgtgcccatgataacaatgtccaagacatcattgcggagatcattaagaaagaaatcaacccgaaaagcgatggtttcaccatcactgacgccgaaatgaaacaagcgttcgagatttacaataaggacatgctgagcagcgacaagaagtacaccctgaataacatcccggcagcttatgccgtgatgttgcagaacatggaaacgattacccgtgtctattatggtgacctgtacaccgacgacggccactacatggaaaccaagtccccgtattacgacaccatcgttaacctgatgaaaagccgtatcaagtacgtcagcggtggccaggcccaacgtagctactggctgccgaccgacggcaagatggacaatagcgacgttgagctgtatcgcaccaacgaagtgtataccagcgtccgttacggtaaagacattatgaccgcgaacgataccgagggtagcaagtacagccgcaccagcggccaggtcaccctggttgcaaacaacccgaagctgaccctggaccagagcgcgaagctgaatgtggaaatgggtaagattcacgcgaatcagaaataccgtgccctgattgtgggcacggctgacggtatcaagaatttcaccagcgacgcagatgctatcgcggcaggctacgtgaaagaaaccgactccaatggcgttctgacttttggcgctaatgacatcaaaggttatgaaaccttcgacatgtccggctttgttgctgtttgggtgccggtcggcgcgagcgatgatcaggacattcgtgtcgctcctagcactgaggccaagaaagagggtgaattgaccctgaaagcgaccgaagcatacgattcccagctgatctatgaaggttttagcaattttcaaaccatcccggatggtagcgacccgagcgtgtacaccaatcgcaagatcgcagagaacgtggacctgttcaagtcctggggtgttacctcgtttgaaatggcaccgcagttcgtttccgcagatgatggcacttttctggactctgtgatccaaaacggctatgcgtttgccgatcgttacgatttggcgatgagcaagaacaacaaatacggcagcaaagaggacttgcgtgacgcgctgaaagccctgcataaagcaggcatccaggcgattgcagactgggtcccggaccagatttatcagttgccgggcaaagaagtggtcacggcgactcgcaccgacggcgcaggccgtaaaatcgcggacgcgatcattgatcatagcctgtacgttgcgaacactaagagcagcggcaaagattaccaggcgaagtacggtggtgagttcttggcggagctgaaggccaagtacccggagatgttcaaagtgaacatgatttctaccggcaaaccgattgatgacagcgtcaaactgaaacagtggaaagcagaatactttaacggcaccaacgtcttggagcgcggtgtgggttatgtcctgagcgatgaagccacgggtaaatactttaccgtcacgaaggatggcaacttcattccgttgcagctgacgggtaatgagaaagtcgtgaccggctttagcaatgatggcaaaggtatcacctacttcggtacgagcggcactcaagcgaaatctgcgttcgttacgttcaatggtaatacttactattttgacgctcgtggtcacatggttacgaacggcgagtattcgccgaacggtaaggatgtttaccgtttcctgccgaatggtattatgctgtctaacgctttttacgttgatgcaaatggtaacacgtacctgtacaacagcaagggccaaatgtacaaaggcggttacaccaaatttgacgttaccgaaacggacaaagatggtaaggaaagcaaggtggtgaagtttcgttactttacgaacgaaggtgtcatggcaaaaggcgttaccgtgattgacggcttcacgcaatactttggtgaagatggtttccaagcgaaagacaagctggtcacgttcaagggcaagacgtactacttcgatgcacacaccggcaatgcgatcaaggacacctggcgtaatatcaatggcaagtggtatcatttcgacgcgaacggcgttgcagcgaccggcgctcaggtcatcaatggccaaaaactgtatttcaacgaggacggcagccaagtgaaaggcggtgttgtcaaaaacgcggacggtacgtattctaaatacaaagagggttctggtgaactggttaccaacgagttcttcacgacggatggcaatgtttggtactacgcaggcgcgaatggcaagaccgttacgggtgcccaggtgattaacggccaacacctgtacttcaatgcggacggttcgcaagtgaagggcggtgtggtcaagaacgcggatggcacctatagcaaatatgatgcgtctaccggcgaacgcctgaccaatgagtttttcaccacgggtgataacaactggtactacattggcgcaaacggcaagagcgtgacgggcgaggtcaagatcggtgacgatacctatttctttgccaaagatggcaagcaagttaagggtcaaactgtcagcgcgggtaacggtcgtattagctactactatggtgatagcggtaagcgtgcggtgagcacttggatcgaaatccaaccgggtgtttatgtctacttcgacaagaacggcattgcctatccgcctcgtgtgctgaattaa

SEQ ID NO: 4 - protein, 1341 aa, Streptococcus salivarius [sequence omitted]

SEQ ID NO: 5 - DNA, 3744 bp, Streptococcus salivarius
  source 1..3744: mol_type = unassigned DNA
atgccaagccacattaagaccatcaacggcaaacaatactacgtggaggatgacggtacgattcgcaagaattacgtcctggagcgtatcggtggcagccaatactttaatgcagaaaccggtgaactgtctaatcagaaagagtatcgtttcgacaaaaatggtggtactggtagcagcgcggacagcacgaacaccaacgtgactgtgaacggtgacaaaaacgcattttacggtaccacggacaaagacattgagctggtcgacggctatttcaccgcgaacacctggtatcgcccgaaagaaatcctgaaagacggcaaagaatggaccgccagcacggagaacgataaacgcccgctgctgaccgtctggtggcctagcaaagcaatccaggcgtcttatctgaactacatgaaagagcaaggcctgggtaccaaccaaacgtacacgagcttctccagccaaacccaaatggatcaagcagccctggaagtgcaaaagcgtattgaagagcgcatcgcacgcgagggcaataccgactggctgcgcacgaccatcaagaacttcgtgaaaacccaaccgggttggaacagcacctctgaaaatctggacaataatgatcatctgcaaggtggcgccctgctgtacaataacgactcccgcacgagccacgcgaacagcgactatcgcctgctgaatcgtacgccgaccagccagaccggcaaacacaatccgaaatacaccaaagataccagcaatggtggtttcgaatttctgctggcgaacgacatcgataactctaatccggcggttcaagcagagcaactgaactggctgcattacattatgaacatcggtaccatcacgggcggttctgaggatgaaaacttcgacggcgttcgtgttgacgctgtggataatgtgaatgcggatctgctgcaaatcgcgagcgactatttcaaagcaaaatacggtgctgatcaaagccaagatcaggcgatcaaacacttgagcatcctggaagcgtggtcccataacgacgcctactataacgaagataccaaaggcgcgcagttgccgatggatgatccgatgcacctggctctggtctactcgctgctgcgtccgatcggcaatcgcagcggtgtggaaccgctgatttccaacagcctgaatgaccgtagcgagtccggtaagaacagcaaacgtatggcgaactacgcgttcgtacgcgcgcatgatagcgaggtgcaatcgattattggccagatcatcaaaaacgagatcaatccgcaaagcaccggtaatacgttcaccctggatgagatgaagaaagcgtttgagatttacaacaaggatatgcgtagcgcgaataagcagtatacgcagtacaacatcccgagcgcgtatgcgttgatgctgacccacaaggataccgttccgcgtgtgtattacggtgatatgtatacggacgacggtcagtacatggcgcaaaagagcccatactatgatgcgatcgaaacgctgctgaaaggtcgcatccgctatgccgcaggtggtcaggacatgaaggtcaactatattggttacggtaacactaacggctgggatgctgcgggcgtgctgaccagcgtacgttatggcacgggcgcaaatagcgccagcgatacgggtaccgccgaaacgcgtaatcaaggtatggcagtgattgttagcaaccaaccggcgctgcgtctgactagcaatttgaccattaacatgggtgccgcacaccgtaatcaggcttaccgtccgctgctgctgacgaccaacgatggcgtcgcgacctatttgaacgatagcgatgcgaatggtatcgttaagtacaccgacggtaatggtaatctgaccttctccgcaaacgagattcgtggcatccgtaacccgcaagttgatggctatctggccgtctgggttccggtaggtgcgtcggagaatcaggatgttcgtgtggcgccgagcaaagagaagaacagctccggtctggtttacgagagcaatgctgccctggatagccaagttatctacgaaggcttcagcaacttccaggacttcgttcagaatccgagccagtataccaacaaaaagattgcagagaatgcaaatttgttcaaatcctggggtattaccagctttgaatttgcgccgcagtacgtgagctcggatgatggtagcttcctggacagcgttattcagaacggttatgcgtttacggaccgctacgacattggtatgagcaaagacaacaaatatggttcgctggcggatttgaaggcagcactgaagagcttgcatgccgttggtattagcgcaatcgcggattgggttcctgatcagatctacaatctgccaggcgacgaggtcgtcaccgcaacccgcgttaacaactacggcgaaaccaaagatggtgcaatcattgatcactctttgtacgcggccaaaacccgtacttttggtaacgactaccagggtaagtatggtggtgcgttcctggacgagctgaaacgtctgtatccgcagatctttgaccgcgttcagatttctaccggtaagcgcatgaccacggacgagaagatcacccaatggtctgcaaagtatatgaacggtacgaacatcttggaccgtggctctgaatacgttttgaagaatggtctgaatggttactatggcaccaatggtggcaaagtttcgctgccgaaagttgtgggtagcaatcaaagcacgaatggcgacaatcaaaacggcgacggtagcggcaagtttgaaaagcgtctgttcagcgtgcgttaccgttataacaatggccagtacgcgaaaaatgcctttatcaaagataacgacggcaatgtttactatttcgacaatagcggtcgtatggctgtcggtgagaaaacgattgacggcaagcagtacttcttcctggctaatggcgttcagctgcgtgacggctaccgtcaaaatcgtcgcggtcaggtgttttactacgaccagaatggtgtgctgaacgcaaacggtaaacaagacccgaagcctgacaacaataacaatgcgagcggccgtaatcaattcgtccagatcggtaacaacgtgtgggcgtattatgatggcaatggtaaacgtgtcaccggtcaccagaacatcaacggtcaggagttgtttttcgataacaacggtgtccaggttaagggtcgtacggtgaatgagaacggtgcaattcgctactatgacgcgaatagcggtgagatggcacgcaatcgtttcgcggagattgaaccgggcgtctgggcatactttaacaatgacggcaccgcagtgaagggttctcagaatatcaatggtcaagacctgtacttcgaccagaacggtcgtcaggtcaagggtgcgctggccaatgttgatggcaacctgcgctattacgacgttaacagcggtgagctgtaccgtaatcgtttccacgaaatcgacggcagctggtattactttgatggtaacggtaatgcggtgaagggtatggtcaatatcaacggccaaaatctgttgtttgacaataacggcaaacagattaagggtcatctggtccgcgtcaacggcgtcgtgcgctattttgatccgaactctggtgaaatggcggttaatcgttgggttgaggtgagcccaggttggtgggtttactttgacggtgaaggtcgtggtcagatctaa

SEQ ID NO: 6 - protein, 1247 aa, Streptococcus salivarius
MPSHIKTINGKQYYVEDDGTIRKNYVLERIGGSQYFNAETGELSNQKEYRFDKNGGTGSSADSTNTNVTVNGDKNAFYGTTDKDIELVDGYFTANTWYRPKEILKDGKEWTASTENDKRPLLTVWWPSKAIQASYLNYMKEQGLGTNQTYTSFSSQTQMDQAALEVQKRIEERIAREGNTDWLRTTIKNFVKTQPGWNSTSENLDNNDHLQGGALLYNNDSRTSHANSDYRLLNRTPTSQTGKHNPKYTKDTSNGGFEFLLANDIDNSNPAVQAEQLNWLHYIMNIGTITGGSEDENFDGVRVDAVDNVNADLLQIASDYFKAKYGADQSQDQAIKHLSILEAWSHNDAYYNEDTKGAQLPMDDPMHLALVYSLLRPIGNRSGVEPLISNSLNDRSESGKNSKRMANYAFVRAHDSEVQSIIGQIIKNEINPQSTGNTFTLDEMKKAFEIYNKDMRSANKQYTQYNIPSAYALMLTHKDTVPRVYYGDMYTDDGQYMAQKSPYYDAIETLLKGRIRYAAGGQDMKVNYIGYGNTNGWDAAGVLTSVRYGTGANSASDTGTAETRNQGMAVIVSNQPALRLTSNLTINMGAAHRNQAYRPLLLTTNDGVATYLNDSDANGIVKYTDGNGNLTFSANEIRGIRNPQVDGYLAVWVPVGASENQDVRVAPSKEKNSSGLVYESNAALDSQVIYEGFSNFQDFVQNPSQYTNKKIAENANLFKSWGITSFEFAPQYVSSDDGSFLDSVIQNGYAFTDRYDIGMSKDNKYGSLADLKAALKSLHAVGISAIADWVPDQIYNLPGDEVVTATRVNNYGETKDGAIIDHSLYAAKTRTFGNDYQGKYGGAFLDELKRLYPQIFDRVQISTGKRMTTDEKITQWSAKYMNGTNILDRGSEYVLKNGLNGYYGTNGGKVSLPKVVGSNQSTNGDNQNGDGSGKFEKRLFSVRYRYNNGQYAKNAFIKDNDGNVYYFDNSGRMAVGEKTIDGKQYFFLANGVQLRDGYRQNRRGQVFYYDQNGVLNANGKQDPKPDNNNNASGRNQFVQIGNNVWAYYDGNGKRVTGHQNINGQELFFDNNGVQVKGRTVNENGAIRYYDANSGEMARNRFAEIEPGVWAYFNNDGTAVKGSQNINGQDLYFDQNGRQVKGALANVDGNLRYYDVNSGELYRNRFHEIDGSWYYFDGNGNAVKGMVNINGQNLLFDNNGKQIKGHLVRVNGVVRYFDPNSGEMAVNRWVEVSPGWWVYFDGEGRGQI

SEQ ID NO: 7 - DNA, 4434 bp, Streptococcus salivarius
  source 1..4434: mol_type = unassigned DNA
atggacgaaacgcaggataagaccgtgacgcagagcaacagcggcaccaccgcttccctggtcactagccctgaagccacgaaagaggcggacaaacgcacgaacactaaagaggccgacgttctgacgcctgcaaaagaaacgaacgcagtcgagactgcgaccaccactaacacccaggcgacggcggaggccgccacgaccgcgaccaccgcggacgtcgcggtggctgcggtgccgaacaaagaagcggtcgttaccacggatgctccggcggtcacgaccgagaaagcggaagaacagccggctaccgttaaagcagaagtcgtcaatacggaagtgaaagcgccggaagcggctctgaaagacagcgaggttgaggcagcgctgagcctgaagaacatcaagaacattgatggcaagtattactatgttaatgaggatggcagccacaaagagaatttcgctattaccgtgaatggccagctgctgtactttggtaaagacggtgcgctgacgtcctctagcacgtattcttttaccccaggcactaccaatatcgtggacggttttagcattaacaaccgcgcttacgacagcagcgaggcgagctttgagctgatcgacggttacttgaccgcagacagctggtatcgtccggctagcatcatcaaagatggtgttacgtggcaagcgtccaccgccgaggattttcgtccgctgctgatggcatggtggccgaatgtggatacgcaggtgaactatttgaattacatgtccaaagttttcaacctggacgcgaaatactctagcaccgacaaacaggaaaccctgaaagtggcagcaaaagacattcaaatcaagattgaacaaaagattcaagcggagaagagcacgcagtggctgcgtgaaactatcagcgcctttgtgaaaacccagccgcagtggaacaaagaaaccgagaattacagcaagggtggtggtgaggaccacctgcaaggtggcgcactgctgtatgttaacgacagccgtaccccttgggcgaatagcgattaccgtcgtctgaatcgcaccgcaaccaatcagacgggcacgatcgataagtctattctggacgagcagtctgacccaaaccacatgggcggtttcgactttctgctggcgaacgacgtcgacctgagcaatccggtcgtgcaggctgagcagctgaatcaaatccactatctgatgaattggggttccattgtgatgggtgacaaggatgcgaactttgacggcattcgtgtcgatgcagttgacaacgtggacgcggacatgttgcaactgtataccaattacttccgtgagtactacggtgtgaacaagagcgaagctaacgcactggctcacatcagcgttctggaggcgtggagcctgaatgataatcattacaatgacaagaccgatggtgcggcactggcaatggagaataagcaacgtctggcgctgttgttttcgttggcgaaaccgatcaaagagcgtaccccggcagtgagcccgctgtataacaacaccttcaataccacccagcgtgatgaaaagaccgattggattaacaaagacggtagcaaggcttacaacgaagatggcacggtcaaacaatcgaccatcggtaagtacaacgagaaatacggtgacgcatccggtaactacgttttcatccgtgcccacgataacaacgtccaggacatcatcgccgagatcatcaagaaagagatcaacccgaaaagcgacggcttcaccatcaccgacgccgaaatgaagcaagcctttgaaatctataacaaagatatgctgtcgagcgacaaaaagtataccctgaataacattccggcagcgtatgccgtgatgttgcagaatatggaaacgattacccgcgtctattacggtgatctgtatacggacgacggtcactacatggaaaccaaatctccgtattacgataccatcgtgaatttgatgaagagccgtatcaagtatgtttcgggtggccaggcgcaacgtagctattggctgccgaccgacggtaagatggacaatagcgacgttgagctgtaccgcacgaatgaggtttacacgagcgtgcgctatggtaaggatatcatgaccgctaatgataccgaaggctctaagtattcccgcaccagcggccaagtcaccttggtcgcgaacaatccgaagctgaatctggaccaaagcgccaagttgaatgtggagatgggcaaaatccatgcgaatcagaagtatcgcgcactgattgtcggcactgcggacggcattaagaactttacttccgacgcggacgccattgcagcgggttatgtgaaagaaaccgatagcaacggcgtgctgaccttcggtgctaacgacattaagggctacgaaacgtttgatatgagcggtttcgtggcggtgtgggttccggtgggtgcatctgacaatcaggacattcgtgttgcgccgagcaccgaggcaaagaaagaaggtgagctgaccttgaaggcgacggaagcgtatgatagccagctgatttacgaaggctttagcaatttccagacgatcccagatggcagcgatccgtccgtgtatacgaaccgcaagattgcggagaacgtggatctgttcaaaagctggggtgtcaccagctttgagatggcaccgcaatttgtctcggcggatgatggcacctttctggatagcgttattcagaatggctacgccttcgccgaccgttatgacctggccatgtccaagaacaacaagtatggtagcaaagaggacctgcgtgatgcactgaaagcactgcataaggcgggtattcaagctatcgcagactgggttccagaccagatctaccagctgccgggcaaagaagttgtcaccgccacccgtacggatggtgctggccgtaagatcgcagacgcgattatcgaccattctctgtatgttgcaaacagcaaaagcagcggcaaagattatcaagcaaagtacggtggcgagttcctggccgagctgaaagccaaatacccggaaatgttcaaagttaacatgattagcacgggtaagccgattgatgactccgtgaaattgaagcaatggaaagccgagtacttcaatggcaccaacgttttggaacgtggtgtcggctatgttctgagcgacgaggcgaccggtaagtatttcacggtgaccaaagaaggcaatttcattccgctgcaactgacgggtaaagagaaagttatcacgggtttctccagcgatggtaagggtatcacctatttcggtacgagcggtacgcaggcgaagtctgcgtttgttaccttcaatggtaacacctactatttcgacgcgcgtggccacatggttaccaatagcgaatacagcccgaatggcaaggacgtctaccgttttctgccgaacggtatcatgctgagcaatgcgttttacattgatgcgaacggtaatacctacctgtacaactctaagggtcaaatgtacaaaggcggttacacgaaattcgatgtttctgaaacggataaggacggtaaagagtccaaggtcgtcaagttccgctactttacgaacgaaggcgtcatggccaagggtgttaccgtcattgatggttttacccaatacttcggtgaggacggctttcaagcgaaggataagctggtcaccttcaagggcaagacgtattacttcgacgcacacactggtaatggtatcaaagatacctggcgcaatatcaatggtaaatggtactatttcgacgcgaatggcgttgctgcgaccggtgcgcaggtgattaacggccagaaactgtacttcaacgaggatggctcccaagtcaaaggcggcgtggttaagaacgcagacggcacctatagcaaatacaaagaaggttttggtgagctggttactaacgagtttttcacgactgatggcaatgtttggtactacgccggtgcaaatggtaaaaccgttaccggtgcacaagtgatcaacggccaacatttgtacttcaatgcggacggttcccaggtgaagggtggcgttgtcaagaacgcggatggcacctacagcaagtacaatgctagcactggtgaacgtctgacgaacgagttctttacgaccggtgataacaattggtattacattggcgcaaacggtaagagcgtgacgggtgaggtcaagattggtgatgatacttactttttcgcgaaggatggcaaacaagttaaaggtcaaaccgtcagcgccggtaatggtcgcattagctactactacggtgacagcggcaagcgtgcggttagcacctggattgagattcagccgggtgtttatgtgtatttcgacaaaaacggtttggcgtaccctccgcgtgttctgaattaa

SEQ ID NO: 8 - protein, 1477 aa, Streptococcus salivarius
MDETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 9 - DNA, 4311 bp, Streptococcus downei
  source 1..4311: mol_type = unassigned DNA
atggttgacggcaaatactactactacgatcaggacggcaacgtaaagaaaaacttcgcggttagcgtgggcgagaaaatctattactttgacgaaactggcgcctacaaagacaccagcaaagttgaggcggacaaaagcggcagcgacattagcaaggaagagactaccttcgcggcaaacaaccgcgcctacagcaccagcgcggagaattttgaggcgatcgacaattatctgaccgcggactcctggtatcgtcctaaatccatcctgaaggatggcaaaacgtggacggaaagcagcaaagatgactttcgtccgctgctgatggcgtggtggccggataccgaaacgaagcgcaattacgtgaactacatgaacaaagttgttggcatcgacaagacctataccgcggaaaccagccaggccgacttgaccgctgcggcggaactggtgcaagcacgcattgagcagaagatcacgaccgaacagaacacgaaatggctgcgtgaggcaatctcggcatttgttaaaacgcaaccgcagtggaacggtgaaagcgagaagccgtacgacgatcacctgcaaaacggtgctctgaaatttgataatcagagcgacctgaccccggatacgcaaagcaactaccgtctgttgaaccgtaccccgactaatcagacgggtagcctggacagccgcttcacttataacgcgaacgaccctttgggcggttatgagctgctgctggcaaatgacgtcgataacagcaatccgatcgtgcaggcggagcagctgaactggctgcattacctgctgaattttggtacgatctacgccaaagatgccgacgctaacttcgatagcattcgtgtggacgcggttgataacgtcgatgcggatctgctgcaaattagcagcgattacctgaaagcagcctacggcattgataagaataacaaaaacgcgaacaaccacgtgagcattgtcgaagcctggagcgataatgataccccgtacctgcatgacgatggtgacaacctgatgaatatggataacaaatttcgcctgtccatgctgtggtcgctggccaaaccgctggacaagcgtagcggtctgaacccgctgattcataacagcttggtggatcgtgaagttgatgaccgcgaggttgaaacggttccgagctattcttttgcacgtgcgcatgatagcgaggtccaggacttgatccgtgacatcatcaaggcagagatcaatccgaacgcattcggttatagctttacccaagacgagattgaccaggcctttaagatttacaatgaggatctgaagaaaacggataagaaatacacccactataatgtgccgttgagctacaccctgctgctgacgaataagggtagcatcccacgtgtctactatggtgatatgtttaccgacgatggtcagtatatggcgaacaaaaccgtcaactatgacgccattgaatctctgctgaaagcgcgtatgaagtatgtcgctggcggtcaagcaatgcagaactaccaaatcggtaatggtgagatcctgaccagcgttcgttatggtaagggtgccctgaaacagagcgacaaaggtgatgcgaccacgcgcaccagcggtgtcggtgtcgttatgggcaatcagccaaactttagcttggacggcaaagtggtggctctgaacatgggcgcagctcatgcgaatcaggagtatcgtgcgctgatggttagcacgaaagacggtgttgccacgtatgcgaccgatgcagatgcgagcaaagccggtctggtcaaacgtaccgacgaaaacggctacctgtatttcctgaatgacgacctgaagggtgtggccaatcctcaggtgagcggtttcttgcaggtgtgggttccggtgggtgccgcggatgatcaagatatccgtgttgcagctagcgataccgcatccaccgatggcaagagcctgcaccaagacgccgcgatggatagccgtgttatgtttgaaggcttctctaactttcagtcctttgccacgaaagaagaggaatataccaacgtcgttatcgccaacaatgtggataagttcgttagctggggtatcacggatttcgagatggccccacaatatgtttccagcaccgacggtcaattcctggactctgtcattcagaacggttatgcttttacggaccgttatgacttgggcatgtctaaggcaaacaaatacggcacggccgatcaactggttaaggccattaaggccctgcacgcgaagggcctgaaggttatggcagattgggtgccggatcagatgtataccttcccgaaacaggaagtcgtgaccgttacccgtaccgacaaatttggcaaaccgatcgcaggttcccaaatcaatcatagcctgtatgttaccgataccaagtccagcggcgatgactatcaggccaaatatggtggtgcgtttctggacgagctgaaggagaaatatccggagctgttcacgaagaaacaaatcagcacgggtcaagctattgacccgagcgtgaaaatcaaacagtggtctgctaagtatttcaatggctccaacatcctgggtcgcggtgcggactacgtactgtcggatcaggcgagcaacaaatacctgaacgtgtctgacgataaactgttcctgccgaaaaccttgctgggccaagttgtcgagagcggtatccgctttgacggcactggttatgtgtacaactctagcactacgggtgaaaaagttaccgattccttcattacggaggcaggtaatctgtactacttcggtcaagacggctatatggtgaccggcgcacagaacattaagggcagcaactattacttcctggccaatggtgcggccctgcgtaacaccgtttacaccgatgcgcaaggtcagaatcactattacggcaacgacggcaagcgttatgagaatggttaccaacagttcggcaacgattcttggcgttacttcaaaaatggcgtgatggcgctgggtctgactacggtggatggtcacgtgcagtatttcgataaagatggtgtccaggccaaggataagatcattgtcacccgcgatggcaaagtccgctatttcgaccagcacaacggtaatgcggttactaacacgttcgttgcggacaagacgggtcactggtactatctgggcaaagacggcgtcgcggttaccggtgcgcagactgtgggtaaacagcatttgtactttgaagcgaacggtcaacaagtcaagggtgacttcgtgacggctaaagacggtaaactgtacttctatgatgtggacagcggcgacatgtggaccaatacctttatcgaggataaagcgggtaattggttctacctgggtaaggacggtgcggccgtcaccggtgcacagacgatcaaaggccagaaattgtatttcaaagccaacggtcagcaagttaaaggtgacattgtcaaggacgcggacggtaagatccgttattacgacgctcagaccggtgaacaggtctttaacaagtccgttagcgtcaacggtaagacctactatttcggtagcgacggcaccgcgcaaacccaggcgaatccgaaaggccaaacctttaaggatggtagcggcgttctgcgtttctacaatttggagggccagtatgtctcgggcagcggctggtacgaaacggccgagcacgagtgggtatatgtgaaatccggtaaagttctgaccggtgcccagacgattggtaatcaacgtgtttacttcaaggacaatggtcaccaggtgaaaggccagctggtcacgggtaatgacggtaaattgcgttactacgacgcgaacagcggtgatcaagcattcaacaaatccgtcacggttaacggtaaaacctactactttggcagcgatggtacggcgcagacgcaggctaatcctaagggtcagaccttcaaagatggtagcggcgtgctgcgtttttacaacttggaaggccaatacgtgtctggcagcggttggtacaagaatgcgcagggccagtggctgtacgtgaaagatggcaaggtcctgaccggtctgcaaacggtcggcaatcagaaggtctacttcgacaaaaatggcatccaagcaaagggtaaggccgttcgcacgtccgatggtaaagtgcgctactttgatgagaatagcggtagcatgattacgaaccaatggaagttcgtttacggtcaatactattacttcggttctgacggcgcagcggtttaccgtggttggaactaa

SEQ ID NO: 10 - protein, 1436 aa, Streptococcus downei
MVDGKYYYYDQDGNVKKNFAVSVGEKIYYFDETGAYKDTSKVEADKSGSDISKEETTFAANNRAYSTSAENFEAIDNYLTADSWYRPKSILKDGKTWTESSKDDFRPLLMAWWPDTETKRNYVNYMNKVVGIDKTYTAETSQADLTAAAELVQARIEQKITTEQNTKWLREAISAFVKTQPQWNGESEKPYDDHLQNGALKFDNQSDLTPDTQSNYRLLNRTPTNQTGSLDSRFTYNANDPLGGYELLLANDVDNSNPIVQAEQLNWLHYLLNFGTIYAKDADANFDSIRVDAVDNVDADLLQISSDYLKAAYGIDKNNKNANNHVSIVEAWSDNDTPYLHDDGDNLMNMDNKFRLSMLWSLAKPLDKRSGLNPLIHNSLVDREVDDREVETVPSYSFARAHDSEVQDLIRDIIKAEINPNAFGYSFTQDEIDQAFKIYNEDLKKTDKKYTHYNVPLSYTLLLTNKGSIPRVYYGDMFTDDGQYMANKTVNYDAIESLLKARMKYVAGGQAMQNYQIGNGEILTSVRYGKGALKQSDKGDATTRTSGVGVVMGNQPNFSLDGKVVALNMGAAHANQEYRALMVSTKDGVATYATDADASKAGLVKRTDENGYLYFLNDDLKGVANPQVSGFLQVWVPVGAADDQDIRVAASDTASTDGKSLHQDAAMDSRVMFEGFSNFQSFATKEEEYTNVVIANNVDKFVSWGITDFEMAPQYVSSTDGQFLDSVIQNGYAFTDRYDLGMSKANKYGTADQLVKAIKALHAKGLKVMADWVPDQMYTFPKQEVVTVTRTDKFGKPIAGSQINHSLYVTDTKSSGDDYQAKYGGAFLDELKEKYPELFTKKQISTGQAIDPSVKIKQWSAKYFNGSNILGRGADYVLSDQASNKYLNVSDDKLFLPKTLLGQVVESGIRFDGTGYVYNSSTTGEKVTDSFITEAGNLYYFGQDGYMVTGAQNIKGSNYYFLANGAALRNTVYTDAQGQNHYYGNDGKRYENGYQQFGNDSWRYFKNGVMALGLTTVDGHVQYFDKDGVQAKDKIIVTRDGKVRYFDQHNGNAVTNTFVADKTGHWYYLGKDGVAVTGAQTVGKQHLYFEANGQQVKGDFVTAKDGKLYFYDVDSGDMWTNTFIEDKAGNWFYLGKDGAAVTGAQTIKGQKLYFKANGQQVKGDIVKDADGKIRYYDAQTGEQVFNKSVSVNGKTYYFGSDGTAQTQANPKGQTFKDGSGVLRFYNLEGQYVSGSGWYETAEHEWVYVKSGKVLTGAQTIGNQRVYFKDNGHQVKGQLVTGNDGKLRYYDANSGDQAFNKSVTVNGKTYYFGSDGTAQTQANPKGQTFKDGSGVLRFYNLEGQYVSGSGWYKNAQGQWLYVKDGKVLTGLQTVGNQKVYFDKNGIQAKGKAVRTSDGKVRYFDENSGSMITNQWKFVYGQYYYFGSDGAAVYRGWN

SEQ ID NO: 11 - DNA, 3942 bp, Streptococcus mutans
  source 1..3942: mol_type = unassigned DNA
atgattgacggcaaatactactactatgacaacaacggcaaagtacgcaccaatttcacgttgatcgcggacggtaaaatcctgcattttgatgaaactggcgcgtacaccgacactagcattgataccgtgaacaaggatattgtcacgacgcgtagcaacctgtataagaaatacaatcaagtgtatgatcgcagcgcgcagagcttcgagcatgttgatcactacctgacggcggaatcttggtaccgtccgaaatacattctgaaagatggcaagacctggacccagagcaccgagaaggacttccgtcctctgctgatgacctggtggccgagccaggaaacgcagcgccagtatgtcaacttcatgaacgcccagttgggtatcaacaaaacgtacgacgacaccagcaatcagctgcaattgaacatcgctgctgcaacgatccaagcaaagatcgaagccaaaatcacgacgctgaagaacaccgattggctgcgtcaaacgatcagcgcgttcgtcaaaacccaaagcgcttggaatagcgacagcgaaaagccgtttgatgaccatctgcaaaacggtgcggttctgtatgataacgaaggtaaattgacgccgtatgccaatagcaactatcgtattctgaaccgcacgccgaccaaccagaccggtaagaaggacccgcgttataccgccgacaacacgatcggcggctacgagtttctgctggccaacgacgtggataatagcaacccggtggttcaggccgagcagctgaactggctgcacttcctgatgaactttggtaatatctacgcaaacgaccctgacgctaacttcgactccatccgcgttgacgctgtcgataatgtggacgccgatctgttacagatcgcgggtgactatctgaaagcggcaaagggcatccataagaatgacaaagcggcgaacgaccacctgtccattctggaagcgtggagcgacaatgacactccgtatctgcatgatgatggcgacaacatgattaacatggataacaaactgcgcctgagcctgctgttctccctggcgaaaccgctgaatcagcgtagcggtatgaacccgttgattacgaacagcctggtcaaccgtactgatgataatgccgaaacggcggcagtgccaagctactcttttatccgtgcccacgatagcgaggtccaggatttgattcgtgatatcattaaggctgagattaacccgaacgtcgtcggttacagcttcacgatggaagagattaagaaggcatttgagatctacaataaggacctgttggccacggagaagaagtatacccactataacaccgcattgagctacgcgttgctgctgacgaacaagagcagcgtgccgcgtgtctactatggtgatatgtttacggacgatggtcaatacatggcccacaagaccattaactacgaggcaatcgaaaccctgctgaaagcacgtatcaagtacgtgtccggtggtcaggctatgcgcaaccagcaagtgggtaattcggagatcatcaccagcgtgcgttacggtaaaggtgcgctgaaggcgatggatacgggtgaccgcactacccgtacctctggtgtggcggtcattgagggcaacaacccgagcttgcgcctgaaggcttctgatcgtgtggttgtgaatatgggtgcggcccacaaaaatcaagcctatcgcccgctgctgttgacgaccgataacggcattaaggcctatcacagcgaccaagaagcggcaggcctggtgcgttacaccaacgaccgtggcgaactgatctttaccgcagccgacattaagggctacgcaaatccgcaagttagcggctacctgggcgtctgggtccctgttggcgcagcagctgatcaggacgttcgtgttgcggcgagcaccgcgccaagcacggacggcaagagcgttcaccagaacgcggctctggacagccgtgtgatgttcgagggtttctcgaacttccaggcatttgctaccaagaaagaagagtataccaatgtggtcatcgctaagaatgtggataagttcgcggagtggggtgtcaccgatttcgagatggctccgcaatacgtttctagcaccgacggtagctttttggatagcgtgattcaaaacggttatgcttttaccgaccgttacgacctgggcatcagcaagccgaacaaatatggcaccgcggacgatctggttaaagcgattaaggcattgcacagcaaaggcatcaaagttatggcggattgggttccggaccagatgtatgccctgccggaaaaagaggttgtgacggcaacccgtgttgacaaatacggtacgccggtagctggcagccagatcaaaaacacgctgtacgtggtcgatggtaaatctagcggtaaggaccagcaggcgaagtacggtggtgccttcctggaagagctgcaagcgaagtatccggaactgttcgcgcgcaaacagattagcaccggtgttccgatggacccgagcgtcaagattaagcaatggagcgcaaaatacttcaacggcacgaatatcctgggtcgtggtgctggttacgtgctgaaagatcaggcaaccaacacctactttaacatcagcgacaataaagagatcaatttcctgccaaagacgttgctgaaccaggattctcaagttggctttagctacgacggtaagggctatgtgtactacagcacctcgggctaccaggctaaaaacacgttcatcagcgagggtgacaagtggtattacttcgacaataacggttatatggttaccggcgcacagagcattaatggtgtgaactattacttcctgccgaatggtttacagctgcgtgatgcgattctgaaaaatgaggacggtacgtacgcgtattatggcaatgatggtcgccgctacgagaatggctattatcagtttatgagcggtgtttggcgccatttcaataatggcgagatgtccgttggtctgaccgtcattgacggtcaagttcaatactttgacgagatgggttaccaggcgaaaggcaaattcgttaccaccgcggatggtaagatccgttacttcgataagcagagcggcaatatgtatcgtaatcgtttcattgagaacgaagagggcaaatggctgtacctgggtgaggacggcgcggcagtcaccggtagccagacgatcaatggtcagcacctgtattttcgtgctaacggcgttcaggttaagggtgagttcgtgaccgatcgtcatggccgcatctcttattacgacggcaacagcggtgatcagatccgcaaccgtttcgtccgcaatgcgcaaggccagtggttttactttgacaacaatggctatgcagtaactggtgctcgtacgatcaacggccagcacctgtatttccgcgcgaacggtgttcaggtaaaaggtgagtttgttacggaccgccacggccgcattagctattatgatggtaatagcggtgaccaaattcgcaatcgtttcgtgcgtaatgcacagggtcagtggttctacttcgacaataatggttatgcagtcacgggtgcacgtaccattaacggccaacacctgtactttcgcgccaatggtgtgcaagtgaaaggcgaatttgttactgatcgttatggtcgtatcagctactatgatggcaattctggcgaccaaattcgcaatcgctttgttcgtaacgcccaaggtcaatggttctatttcgacaacaacggttacgcggtgaccggtgcccgcacgattaatggtcaacacttgtacttccgtgccaacggtgtccaggtgaagggtgaatttgtgaccgaccgctatggtcgcatttcttactacgacgcaaattccggtgaacgcgtccgtatcaattaa

SEQ ID NO: 12 - protein, 1313 aa, Streptococcus mutans
MIDGKYYYYDNNGKVRTNFTLIADGKILHFDETGAYTDTSIDTVNKDIVTTRSNLYKKYNQVYDRSAQSFEHVDHYLTAESWYRPKYILKDGKTWTQSTEKDFRPLLMTWWPSQETQRQYVNFMNAQLGINKTYDDTSNQLQLNIAAATIQAKIEAKITTLKNTDWLRQTISAFVKTQSAWNSDSEKPFDDHLQNGAVLYDNEGKLTPYANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKAMDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFNISDNKEINFLPKTLLNQDSQVGFSYDGKGYVYYSTSGYQAKNTFISEGDKWYYFDNNGYMVTGAQSINGVNYYFLPNGLQLRDAILKNEDGTYAYYGNDGRRYENGYYQFMSGVWRHFNNGEMSVGLTVIDGQVQYFDEMGYQAKGKFVTTADGKIRYFDKQSGNMYRNRFIENEEGKWLYLGEDGAAVTGSQTINGQHLYFRANGVQVKGEFVTDRHGRISYYDGNSGDQIRNRFVRNAQGQWFYFDNNGYAVTGARTINGQHLYFRANGVQVKGEFVTDRHGRISYYDGNSGDQIRNRFVRNAQGQWFYFDNNGYAVTGARTINGQHLYFRANGVQVKGEFVTDRYGRISYYDGNSGDQIRNRFVRNAQGQWFYFDNNGYAVTGARTINGQHLYFRANGVQVKGEFVTDRYGRISYYDANSGERVRIN

SEQ ID NO: 13 - DNA, 3972 bp, Streptococcus dentirousetti
  source 1..3972: mol_type = unassigned DNA
atggttgacggcaaatactactactacgatgcagacggcaacgtaaagaaaaacttcgcggttagcgttggcgatgccattttctattttgatgaaacgggtgcctacaaagataccagcaaagttgatgcggataagaccagctctagcgtcaatcagaccacggaaacgttcgcagcgaataaccgtgcgtatagcaccgcagccgagaactttgaagcgattgataactacctgactgcggatagctggtatcgtccgaagtctatcttgaaagatggtacgacgtggaccgaaagcaccaaggatgattttcgcccgctgctgatggcgtggtggccggataccgaaaccaaacgtaactacgtgaactatatgaacaaggtggtcggtatcgacaaaacgtacaccgcggaaacgtcccaagctgacctgacggcggcagccgaactggtgcaggcgcgtatcgagcagaaaatcactagcgaaaagaatacgaagtggctgcgtgaggcgatttccgcgttcgttaagactcaaccgcagtggaatggcgagagcgagaaaccttatgatgaccacctgcaaaatggtgcgctgaagttcgacaatgaaaccagcctgaccccggatacgcagagcggctatcgcatcctgaaccgtaccccgacgaatcaaaccggtagcctggacccgcgcttcacctttaatcagaatgacccgctgggtggttatgagtatttgctggctaatgatgtcgataacagcaacccggtcgttcaggccgagagcctgaactggctgcattacctgctgaattttggtagcatttacgcgaatgatccggaggccaatttcgacagcatccgtgtggacgcggtggacaatgttgacgcagacctgctgcaaattagctcggattacctgaaatcggcgtacaaaattgacaagaacaacaaaaatgcgaacgaccacgttagcatcgtcgaggcgtggagcgacaatgataccccgtacctgaatgatgatggcgacaatctgatgaacatggataacaagtttcgtctgagcatgctgtggagcctggcgaagccaaccaatgtccgtagcggcttgaatccgctgatccacaacagcgtggttgaccgtgaggtggacgaccgtgaagttgaggctaccccgaattacagctttgcacgcgcacacgacagcgaagttcaagatttgattcgcgacatcatcaaagctgagatcaacccaaacagcttcggttatagctttacccaagaggaaatcgaccaggccttcaagatctacaatgaggatttgaagaaaaccaataagaagtatacccactacaacgtcccgctgagctacaccctgctgctgacgaacaagggcagcattccacgcatttactacggtgacatgtttacggatgacggtcagtatatggccaacaaaaccgttaactatgacgccattgagagcctgctgaaagcacgtatgaagtatgttagcggtggccaagcgatgcagaattacaacatcggcaacggcgagattctgaccagcgtccgttacggtaagggtgccctgaaacagagcgacaaaggcgataagactactcgtaccagcggtattggcgttgtgatgggtaaccagagcaatttcagcctggagggcaaggtggtggccctgaatatgggtgcaacgcataccaaacagaagtatcgtgcattgatggtgtctacggaaaccggcgtggcgatttacaatagcgatgaagaagcagaggcagcaggcctgatcaaaacgaccgatgagaatggttatttgtactttctgaatgacgatctgaagggcgtggctaacccgcaggtcagcggcttcctgcaagtgtgggttccggttggtgcaccggctgaccaggacattcgtgtggcggcgaccgatgcggcttctaccgacggtaagagcctgcatcaggacgcagctctggattctcgcgtcatgtttgaaggtttcagcaacttccagagcttcgcaaccaaggaagaggaatacaccaacgttgttattgcaaagaacgtggataagttcgtgagctggggtatcaccgacttcgagatggcaccgcagtacgttagctctaccgatggcacctttctggatagcgtgattcaaaatggctatgcctttacggaccgttacgacctgggtatgagcaaagcaaacaagtatggtactgctgaccaactggtggccgcgattaaagcgctgcatgcgaagggtctgcgtgtgatggcggattgggtcccagatcaaatgtacactttccctaagaaggaagtggttaccgttacccgtacggacaaatttggcaatccagtggcaggcagccaaatcaaccacaccttgtacgtcactgatactaagggtagcggtgacgactaccaggcgaagtacggtggcgcattcctggatgaactgaaagaaaagtacccggagctgtttaccaagaagcaaatcagcaccggtcaggcaatcgacccgagcgtgaaaatcaagcagtggagcgcgaagtacttcaacggtagcaatatcttgggtcgcggtgcgaactacgtgctgtccgaccaggcgtctaacaagtactttaacgtggccgaaggtaaagtctttctgccagcggcgatgctgggtaaggtcgtcgagagcggtatccgtttcgacggtaaaggttatatctataacagcagcaccactggcgaacaagtgaaggacagcttcattaccgaagcgggtaacttgtactattttggcaaagatggttatatggtcatgggtgcacagaatatccagggtgctaactactacttcttggcgaatggtgcggccctgcgcaatagcatcctgacggatcaggatggcaaaagccactattatgcaaatgacggcaagcgttatgagaacggctactatcaattcggtaacgactcctggcgctattttgaaaacggcgttatggccgttggtttgacgcgcgttgcgggccacgaccaatactttgataaggatggtatccaagcgaagaataagatcattgttacgcgtgacggtaaggtccgctacttcgacgaacacaacggcaatgctgccacgaatacgtttatcagcgatcaagccggccattggtactacctgggtaaagatggtgtcgccgtgacgggtgcgcagaccgttggcaagcaacacctgtacttcgaggctaacggccaacaagtaaaaggcgattttgttaccgccaaggacggtaagttgtattttctggacggtgactctggcgacatgtggaccgataccttcgtccaggataaggctggtcattggttctatctgggcaaagacggtgcggcggtaaccggtgcccagaccgtccgtggtcagaagctgtacttcaaagcgaatggccagcaggttaagggtgacattgtgaaaggcgcggatggtaaaatccgttactatgatgcaaattccggtgaccaggtttacaatcgcacggtgaaaggctccgacggcaagacctatatcattggtaatgacggcgtcgcaatcacgcaaaccatcgccaaaggccagaccatcaaggatggcagcgttctgcgcttctatagcatggagggtcagctggtgaccggcagcggctggtattccaacgcgaaaggtcaatggttgtatgtcaagaacggtcaagtcctgacgggtttgcagacggtgggcagccagcgtgtgtactttgacgcaaatggtattcaagcgaaaggtaaagcagtgcgtacctccgatggcaaactgcgttacttcgatgcgaacagcggcagcatgatcaccaatcagtggaaagaagttaatggtcagtactactatttcgacaacaacggtgttgcgatctatcgcggttggaactaa

SEQ ID NO: 14 - protein, 1323 aa, Streptococcus dentirousetti
MVDGKYYYYDADGNVKKNFAVSVGDAIFYFDETGAYKDTSKVDADKTSSSVNQTTETFAANNRAYSTAAENFEAIDNYLTADSWYRPKSILKDGTTWTESTKDDFRPLLMAWWPDTETKRNYVNYMNKVVGIDKTYTAETSQADLTAAAELVQARIEQKITSEKNTKWLREAISAFVKTQPQWNGESEKPYDDHLQNGALKFDNETSLTPDTQSGYRILNRTPTNQTGSLDPRFTFNQNDPLGGYEYLLANDVDNSNPVVQAESLNWLHYLLNFGSIYANDPEANFDSIRVDAVDNVDADLLQISSDYLKSAYKIDKNNKNANDHVSIVEAWSDNDTPYLNDDGDNLMNMDNKFRLSMLWSLAKPTNVRSGLNPLIHNSVVDREVDDREVEATPNYSFARAHDSEVQDLIRDIIKAEINPNSFGYSFTQEEIDQAFKIYNEDLKKTNKKYTHYNVPLSYTLLLTNKGSIPRIYYGDMFTDDGQYMANKTVNYDAIESLLKARMKYVSGGQAMQNYNIGNGEILTSVRYGKGALKQSDKGDKTTRTSGIGVVMGNQSNFSLEGKVVALNMGATHTKQKYRALMVSTETGVAIYNSDEEAEAAGLIKTTDENGYLYFLNDDLKGVANPQVSGFLQVWVPVGAPADQDIRVAATDAASTDGKSLHQDAALDSRVMFEGFSNFQSFATKEEEYTNVVIAKNVDKFVSWGITDFEMAPQYVSSTDGTFLDSVIQNGYAFTDRYDLGMSKANKYGTADQLVAAIKALHAKGLRVMADWVPDQMYTFPKKEVVTVTRTDKFGNPVAGSQINHTLYVTDTKGSGDDYQAKYGGAFLDELKEKYPELFTKKQISTGQAIDPSVKIKQWSAKYFNGSNILGRGANYVLSDQASNKYFNVAEGKVFLPAAMLGKVVESGIRFDGKGYIYNSSTTGEQVKDSFITEAGNLYYFGKDGYMVMGAQNIQGANYYFLANGAALRNSILTDQDGKSHYYANDGKRYENGYYQFGNDSWRYFENGVMAVGLTRVAGHDQYFDKDGIQAKNKIIVTRDGKVRYFDEHNGNAATNTFISDQAGHWYYLGKDGVAVTGAQTVGKQHLYFEANGQQVKGDFVTAKDGKLYFLDGDSGDMWTDTFVQDKAGHWFYLGKDGAAVTGAQTVRGQKLYFKANGQQVKGDIVKGADGKIRYYDANSGDQVYNRTVKGSDGKTYIIGNDGVAITQTIAKGQTIKDGSVLRFYSMEGQLVTGSGWYSNAKGQWLYVKNGQVLTGLQTVGSQRVYFDANGIQAKGKAVRTSDGKLRYFDANSGSMITNQWKEVNGQYYYFDNNGVAIYRGWN

SEQ ID NO: 15 - DNA, 4047 bp, Streptococcus oralis
  source 1..4047: mol_type = unassigned DNA
atgatcgacggcaaaaactactacgtacaggatgatggcacggtaaagaagaatttcgcggtagaactgaatggtcgtatcctgtattttgatgcagaaaccggcgctctggttgatagcaacgagtatcagttccaacagggtacgagcagcctgaacaatgaattttctcagaagaacgcattctatggtacgaccgataaggatattgagactgtggatggctacctgaccgcagatagctggtatcgcccgaaattcatcctgaaggatggcaagacgtggaccgcgagcacggaaacggatctgcgtccgctgttgatggcatggtggccggacaagcgtacccaaatcaactatctgaactacatgaaccagcagggtctgggtgcgggtgcgtttgagaacaaagtggagcaggccctgctgacgggtgcaagccaacaggtacaacgcaagatcgaagagaagattggtaaagagggtgataccaagtggctgcgcaccctgatgggtgcgttcgtgaaaacgcaaccaaactggaatatcaaaaccgagtctgaaacgaccggcacgaaaaaggaccatctgcaaggcggtgcactgctgtatacgaacaacgagaaatccccgcacgcggacagcaaatttcgtctgctgaatcgtaccccgaccagccaaaccggcacgccgaagtatttcatcgacaagtctaacggtggctacgaatttctgctggcgaacgattttgacaatagcaatcctgcggtacaagctgagcagctgaattggctgcactacatgatgaactttggcagcattgttgcgaatgatccgaccgcgaatttcgacggcgttcgtgtggatgctgttgataacgtcaatgcggacttgttgcaaattgcaagcgattactttaagagccgttacaaagtcggtgagagcgaagaagaagcgatcaagcacctgtccatcctggaagcatggagcgataacgacccggactacaacaaagataccaagggtgcacagttggcgattgataacaaactgcgcctgagcctgctgtactctttcatgcgtaatctgagcatccgtagcggtgttgaaccgacgattaccaatagcctgaatgaccgttccagcgaaaagaagaacggcgagcgtatggcaaattacatcttcgtgcgtgcccacgatagcgaggtccaaacggtgatcgccgacatcattcgcgaaaacatcaatccgaacaccgacggcctgacgtttacgatggacgagctgaagcaggcattcaagatttacaacgaggacatgcgcaaggcggacaaaaagtatacccagtttaacattcctaccgcacacgcgctgatgctgtctaataaggattctattacccgcgtgtactatggtgatctgtatactgacgatggtcagtacatggagaagaaaagcccgtatcacgatgcgattgacgctctgctgcgtgcacgtattaaatacgtcgcgggtggccaggatatgaaagtgacctatatgggcgtgccgcgtgaagcggataagtggagctataacggcattctgaccagcgtgcgctatggcacgggcgctaacgaagccacggatgagggcactgcggaaacgcgcacgcaaggtatggcagtgattgcgagcaataatccaaatctgaaactgaatgaatgggacaagttgcaagtcaacatgggtgcggcgcataagaatcaatattaccgtccggttctgctgaccactaaggacggtatcagccgttatctgaccgatgaagaagtgcctcagagcctgtggaaaaagacggacgcaaacggtattctgaccttcgacatgaatgatattgctggctacagcaacgtgcaagttagcggttacctggccgtctgggtcccggtcggtgcgaaggcggatcaagatgcgcgcacgaccgcatccaagaagaaaaatgcgtcgggtcaggtgtacgaaagcagcgcggctctggatagccagctgatttacgaaggtttcagcaactttcaagactttgccactcgcgatgatcagtacacgaacaaggtcattgcgaaaaacgtgaatctgttcaaagaatggggtgtgaccagcttcgagctgccgccgcagtacgtgagcagccaagatggcacctttctggacagcattatccaaaacggctatgcatttgaagaccgttacgatatggcgatgagcaagaataacaagtatggtagcctgaaagacctgttgaacgcgctgcgcgcactgcacagcgtcaacattcaagcaatcgccgattgggtgccggaccaaatttacaacttgccgggcaaagaggtggtgaccgcaactcgtgtcaacaactacggcacctaccgtgagggtgctgaaatcaaagaaaagctgtatgtcgccaatagcaagaccaacgaaaccgatttccaaggtaaatacggtggtgcgttcctggatgagctgaaggcgaagtacccggagattttcgagcgtgtccaaatcagcaacggccaaaagatgactaccgatgaaaagatcaccaaatggagcgcgaaatactttaatggcaccaatattctgggtcgtggcgcgtactatgtcctgaaagattgggccagcaatgattacctgacgaaccgtaacggcgagattgttttgccgaagcaactggttaacaagaatagctataccggctttgtcagcgacgcgaacggcacgaagttctattctacctctggctaccaggcgaagaacagcttcattcaagacgaaaacggtaattggtattactttgacaaacgtggttatctggttacgggcgcacacgagattgatggcaagcatgtctacttcctgaaaaacggtatccaactgcgtgacagcatccgtgaggatgagaacggtaatcaatactattacgaccagaccggcgcacaagtgctgaaccgttactacacgacggacggtcagaattggcgctatttcgatgcgaaaggtgttatggcacgcggcctggtaaagattggtgacggccaacagtttttcgatgaaaacggttaccaggtcaagggcaagattgttagcgcaaaagacggcaagctgcgctactttgataaagactctggcaatgctgtcattaatcgtttcgcgcagggtgacaatccgagcgactggtactatttcggtgtggaatttgctaaactgacgggtttgcaaaagatcggccagcagacgctgtattttgaccaagacggtaagcaagtcaaaggtaagatcgtaactctgtcggacaaaagcattcgttacttcgatgccaacagcggtgaaatggcggttggcaagttcgcggaaggtgcaaagaatgagtggtattatttcgataaaaccggcaaagcggttactggtttgcagaaaattggtaagcagaccctgtactttgaccaggacggtaaacaggttaaaggcaaggttgtcacgctggctgataaaagcatccgctacttcgacgcagactccggcgagatggcggtcggtaagtttgcagagggtgcgaagaacgagtggtactattttgatcagactggcaaggccgtgactggtttgcaaaagattgacaagcaaaccttgtacttcgaccaggacggtaaacaagtcaagggtaagattgtgacgttgagcgacaagtcgatccgttactttgatgctaatagcggtgagatggctactaacaaattcgtcgagggctcgcagaatgaatggtactacttcgatcaagcgggtaaggctgttacgggcttgcaacaggtcggtcagcaaactctgtacttcacccaggatggtaagcaagtgaagggtaaggtcgtggacgtgaacggtgtttctcgttatttcgacgcaaactccggtgacatggctcgttctaaatggattcaactggaagatggcagctggatgtatttcgaccgtgacggtcgtggccagaattttggccgtaactaa

SEQ ID NO: 16 - protein, 1348 aa, Streptococcus oralis
MIDGKNYYVQDDGTVKKNFAVELNGRILYFDAETGALVDSNEYQFQQGTSSLNNEFSQKNAFYGTTDKDIETVDGYLTADSWYRPKFILKDGKTWTASTETDLRPLLMAWWPDKRTQINYLNYMNQQGLGAGAFENKVEQALLTGASQQVQRKIEEKIGKEGDTKWLRTLMGAFVKTQPNWNIKTESETTGTKKDHLQGGALLYTNNEKSPHADSKFRLLNRTPTSQTGTPKYFIDKSNGGYEFLLANDFDNSNPAVQAEQLNWLHYMMNFGSIVANDPTANFDGVRVDAVDNVNADLLQIASDYFKSRYKVGESEEEAIKHLSILEAWSDNDPDYNKDTKGAQLAIDNKLRLSLLYSFMRNLSIRSGVEPTITNSLNDRSSEKKNGERMANYIFVRAHDSEVQTVIADIIRENINPNTDGLTFTMDELKQAFKIYNEDMRKADKKYTQFNIPTAHALMLSNKDSITRVYYGDLYTDDGQYMEKKSPYHDAIDALLRARIKYVAGGQDMKVTYMGVPREADKWSYNGILTSVRYGTGANEATDEGTAETRTQGMAVIASNNPNLKLNEWDKLQVNMGAAHKNQYYRPVLLTTKDGISRYLTDEEVPQSLWKKTDANGILTFDMNDIAGYSNVQVSGYLAVWVPVGAKADQDARTTASKKKNASGQVYESSAALDSQLIYEGFSNFQDFATRDDQYTNKVIAKNVNLFKEWGVTSFELPPQYVSSQDGTFLDSIIQNGYAFEDRYDMAMSKNNKYGSLKDLLNALRALHSVNIQAIADWVPDQIYNLPGKEVVTATRVNNYGTYREGAEIKEKLYVANSKTNETDFQGKYGGAFLDELKAKYPEIFERVQISNGQKMTTDEKITKWSAKYFNGTNILGRGAYYVLKDWASNDYLTNRNGEIVLPKQLVNKNSYTGFVSDANGTKFYSTSGYQAKNSFIQDENGNWYYFDKRGYLVTGAHEIDGKHVYFLKNGIQLRDSIREDENGNQYYYDQTGAQVLNRYYTTDGQNWRYFDAKGVMARGLVKIGDGQQFFDENGYQVKGKIVSAKDGKLRYFDKDSGNAVINRFAQGDNPSDWYYFGVEFAKLTGLQKIGQQTLYFDQDGKQVKGKIVTLSDKSIRYFDANSGEMAVGKFAEGAKNEWYYFDKTGKAVTGLQKIGKQTLYFDQDGKQVKGKVVTLADKSIRYFDADSGEMAVGKFAEGAKNEWYYFDQTGKAVTGLQKIDKQTLYFDQDGKQVKGKIVTLSDKSIRYFDANSGEMATNKFVEGSQNEWYYFDQAGKAVTGLQQVGQQTLYFTQDGKQVKGKVVDVNGVSRYFDANSGDMARSKWIQLEDGSWMYFDRDGRGQNFGRN

SEQ ID NO: 17 - DNA, 4047 bp, Streptococcus sanguinis
  source 1..4047: mol_type = unassigned DNA
atgattgatggtaaaaagtattacgtacaggacgacggcacggttaagaagaatttcgcggttgagctgaatggcaagatcctgtacttcgatgcagagactggtgcgttgattgacagcgcggagtatcaattccaacaaggcaccagcagcctgaataatgagttcactcaaaagaacgccttttacggtacgaccgataaggatgtggaaaccattgatggttacttgaccgccgattcctggtatcgtccgaagttcattctgaaagatggcaaaacctggacggcgagcacggaaattgacttgcgtccgttgttgatggcgtggtggccggacaaacagacccaggttagctacctgaattacatgaaccagcaaggcttgggtgcaggcgccttcgaaaacaaagtagagcaggcaattctgaccggtgcgtcccaacaggtacaacgtaaaatcgaagaacgcatcggtaaagagggtgataccaagtggctgcgtaccctgatgggtgcatttgtaaagacccagccgaactggaacattaagaccgagtccgaaaccactggcacgaataaagatcatctgcaaggtggcgcactgctgtatagcaattccgacaagacgagccatgccaactctaagtaccgtatcctgaaccgcaccccgaccaaccaaacgggcacgccgaaatactttattgacaagagcaatggtggttatgaatttctgctggcgaatgactttgacaatagcaatccggcagtgcaagcggaacagctgaactggttgcactttatgatgaattttggctccatcgttgcaaatgatccgacggccaacttcgacggcgtccgcgttgacgctgtggataacgtgaatgcggatctgttgcaaattgcgagcgactatttcaagagccgctataaagtcggcgaaagcgaagaagaggccattaagcacctgtccatcctggaagcgtggagcgacaacgacccggactacaacaaggatactaaaggtgcccaactgccgatcgacaacaaactgcgtctgagcctgctgtactccttcatgcgtaagctgagcatccgtagcggcgtcgagccgaccatcaccaactctctgaatgatcgcagcacggagaagaagaatggtgagcgtatggcaaactatatcttcgttcgtgcacatgatagcgaggtgcaaacggtcatcgccgacattatccgtgagaacatcaatccgaataccgacggcctgacgttcacgatggatgaactgaagcaggcctttaaaatttacaatgaggatatgcgtaaagccgacaaaaagtacacgcagttcaatatcccgaccgcgcacgcgctgatgctgagcaacaaagattctatcacccgcgtttactacggtgacctgtatacggatgacggtcagtatatggaaaagaaaagcccgtatcacgacgccattgacgctctgctgcgtgcgcgtatcaaatatgttgcgggtggtcaggacatgaaggtgacctatatgggcgtgccgcgtgaggcagataaatggagctataacggcatcctgaccagcgttcgttatggtacgggtgccaacgaggcaaccgacgagggtacggcagaaacccgtacccagggcatggccgtcattgccagcaacaatccgaacctgaaactgaacgagtgggacaagttgcaggtcaacatgggtgcagctcacaaaaaccaatactatcgtccggtgctgctgaccaccaaggacggcatctcgcgctacctgaccgacgaagaagtcccgcagagcctgtggaaaaagaccgatgcgaacggcatcttgacgtttgacatgaatgatattgcgggttacagcaacgtccaagtgagcggttatctggccgtctgggttcctgtgggtgcgaaggcggaccaggacgctcgtgttacggcatctaagaagaaaaatgcctctggccaagtttacgaaagcagcgcagccctggactcccagctgatctatgagggcttcagcaattttcaggactttgccacccgtgacgaccagtacactaacaaggttatcgcgaaaaacgtcaatctgtttaaagagtggggcgtcaccagcttcgaattgccgccacagtatgtgagcagccaagacggtacgttcctggatagcatcatccagaatggttatgcattcgaagatcgctatgatatggcgatgagcaaaaacaataagtacggtagcttgaacgacctgttgaacgccttgcgtgcactgcatagcgtgaatatccaagcgattgcggattgggtgccggaccagatttacaatctgccgggtaaagaagttgtcactgcaacccgtgttaacaattatggcacgtatcgtgagggtagcgagattaaagagaacctgtacgttgctaacaccaaaaccaatggtacggactaccaaggtaagtatggtggtgcgttcttggacgagctgaaagccaaataccctgagatttttgagcgcgtccaaatcagcaacggccagaagatgaccaccgacgagaagattacgaaatggtccgccaaacactttaacggcacgaacattctgggtcgtggtgcgtattatgtgctgaaagactgggcgagcaacgagtacctgaataacaaaaatggcgagatggttctgccgaagcagctggttaataaaaatgcatataccggcttcgtcagcgacgcgagcggcaccaaatactattctaccagcggctatcaggctcgtaatagctttattcaagatgaaaatggtaattggtactacttcaataaccgtggttatttggtgacgggtgcacaggaaatcgacggtaagcaactgtatttcctgaaaaacggcattcagctgcgtgattctctgcgtgaggacgaaaacggcaaccagtattactatgataagacgggtgcgcaagttctgaatcgttattacactacggacggccaaaattggcgctacttcgacgttaaaggcgtcatggcccgtggtctggtcacgatgggtggtaaccaacaattctttgaccaaaacggttaccaggttaaaggcaaaattgcgcgtgcaaaagacggtaaactgcgttacttcgataaagacagcggtaatgcggcagctaaccgtttcgcccaaggcgataaccctagcgactggtactatttcggtgcagatggtgttgcggttacgggcctgcaaaaggttggtcagcaaactctgtactttgatcaggacggcaagcaggtgaaaggtaaagttgttaccttggcggacaaaagcattcgttatttcgatgcaaacagcggcgagatggcggtgaacaagtttgtggaaggtgctaagaacgtgtggtactacttcgatcaagcaggcaaagcggtgaccggcctgcaaaccatcaataaacaagtgctgtatttcgaccaggatggtaaacaagtcaaaggtaaggtggtcacgctggctgataagtctatccgctacttcgacgcgaacagcggtgagatggcagtgggcaaattcgccgaaggcgcaaagaatgagtggtattactttgaccaggcgggcaaggctgttaccggtctgcaaaagatcggccaacagacgctgtatttcgaccagaacggtaaacaggttaagggtaaagtggtcaccctggcggataagagcatccgctatttcgacgctaactctggcgaaatggcaagcaataagttcgttgagggtgccaaaaatgaatggtactatttcgatcaggctggcaaggcagtgacgggtctgcaacaaattggccagcagaccctgtattttgaccagaatggcaaacaggtgaagggtaagattgtgtatgttaatggtgcgaatcgctactttgatgccaatagcggtgaaatggcgcgtaacaagtggattcagctggaagatggcagctggatgtattttgaccgcaatggtcgtggtcgtcgtttcggttggaactaa

SEQ ID NO: 18 - protein, 1348 aa, Streptococcus sanguinis
MIDGKKYYVQDDGTVKKNFAVELNGKILYFDAETGALIDSAEYQFQQGTSSLNNEFTQKNAFYGTTDKDVETIDGYLTADSWYRPKFILKDGKTWTASTEIDLRPLLMAWWPDKQTQVSYLNYMNQQGLGAGAFENKVEQAILTGASQQVQRKIEERIGKEGDTKWLRTLMGAFVKTQPNWNIKTESETTGTNKDHLQGGALLYSNSDKTSHANSKYRILNRTPTNQTGTPKYFIDKSNGGYEFLLANDFDNSNPAVQAEQLNWLHFMMNFGSIVANDPTANFDGVRVDAVDNVNADLLQIASDYFKSRYKVGESEEEAIKHLSILEAWSDNDPDYNKDTKGAQLPIDNKLRLSLLYSFMRKLSIRSGVEPTITNSLNDRSTEKKNGERMANYIFVRAHDSEVQTVIADIIRENINPNTDGLTFTMDELKQAFKIYNEDMRKADKKYTQFNIPTAHALMLSNKDSITRVYYGDLYTDDGQYMEKKSPYHDAIDALLRARIKYVAGGQDMKVTYMGVPREADKWSYNGILTSVRYGTGANEATDEGTAETRTQGMAVIASNNPNLKLNEWDKLQVNMGAAHKNQYYRPVLLTTKDGISRYLTDEEVPQSLWKKTDANGILTFDMNDIAGYSNVQVSGYLAVWVPVGAKADQDARVTASKKKNASGQVYESSAALDSQLIYEGFSNFQDFATRDDQYTNKVIAKNVNLFKEWGVTSFELPPQYVSSQDGTFLDSIIQNGYAFEDRYDMAMSKNNKYGSLNDLLNALRALHSVNIQAIADWVPDQIYNLPGKEVVTATRVNNYGTYREGSEIKENLYVANTKTNGTDYQGKYGGAFLDELKAKYPEIFERVQISNGQKMTTDEKITKWSAKHFNGTNILGRGAYYVLKDWASNEYLNNKNGEMVLPKQLVNKNAYTGFVSDASGTKYYSTSGYQARNSFIQDENGNWYYFNNRGYLVTGAQEIDGKQLYFLKNGIQLRDSLREDENGNQYYYDKTGAQVLNRYYTTDGQNWRYFDVKGVMARGLVTMGGNQQFFDQNGYQVKGKIARAKDGKLRYFDKDSGNAAANRFAQGDNPSDWYYFGADGVAVTGLQKVGQQTLYFDQDGKQVKGKVVTLADKSIRYFDANSGEMAVNKFVEGAKNVWYYFDQAGKAVTGLQTINKQVLYFDQDGKQVKGKVVTLADKSIRYFDANSGEMAVGKFAEGAKNEWYYFDQAGKAVTGLQKIGQQTLYFDQNGKQVKGKVVTLADKSIRYFDANSGEMASNKFVEGAKNEWYYFDQAGKAVTGLQQIGQQTLYFDQNGKQVKGKIVYVNGANRYFDANSGEMARNKWIQLEDGSWMYFDRNGRGRRFGWN

SEQ ID NO: 19 - DNA, 4023 bp, unidentified
  misc_feature 1..4023: note = unknown Streptococcus sp. C150
  source 1..4023: mol_type = unassigned DNA
atgatcgacggcaaatactactacgtaaacgaggacggcagccacaaagagaatttcgcgatcacggttaatggtcaactgctgtattttggtaaggatggcgcgctgaccagcagcagcacgtacagcttcacccaaggcactaccaatattgtggacggttttagcattaacaaccgtgcgtatgactccagcgaggcctctttcgagctgattgacggttatctgactgcggactcttggtaccgtccggcgagcattatcaaagacggtgtgacgtggcaagcatccaccgccgaggacttccgcccgttgctgatggcgtggtggccgaacgttgatactcaggtgaactacctgaactacatgtccaaagtctttaatctggatgctaaatacagctcgactgataaacaggaaaccctgaaggtggcggcgaaagatatccagatcaaaattgaacaaaagattcaggcggaaaagtccacgcaatggctgcgtgaaacgatcagcgcctttgtaaaaacccagccgcaatggaacaaagagactgagaactacagcaagggcggtggtgaggaccatctgcaaggtggtgccctgctgtatgttaatgactctcgtaccccgtgggcgaacagcaactatcgtttgctgaaccgcacggcgaccaaccagaccggtacgatcgacaagagcatcctggacgagcagagcgatccgaatcacatgggtggttttgatttcttgctggctaatgacgttgacttgagcaatccggtcgtccaggcggaacaactgaatcagatccactacctgatgaattggggttctattgtcatgggtgataaagacgcgaattttgacggtattcgtgtagacgcggtggataatgttgatgcggacatgctgcaattgtacaccaactatttccgcgaatactatggtgtcaacaaaagcgaggcaaacgcgctggcgcacattagcgtcctggaagcctggagcctgaatgacaaccattacaatgataagactgatgttgcggcgctggcaatggagaataagcagcgcttggcactgttgtttagcctggcgaaaccgattaaagaacgcacgcctgccgtgtctccgctgtacaacaatacgtttaacaccactcagcgtgatgaaaagacggactggatcaataaagatggttcgaaagcctacaatgaggatggcactgtcaagaaaagcaccatcggcaagtataacgagaagtatggtgatgctagcggcaactacgttttcatccgcgctcacgacaataacgtgcaagacatcatcgcggagatcattaagaaagagattaacgagaaatctgacggttttaccattacggattcggagatgaagcgtgcatttgagatctataacaaagacatgctgtctaatgacaaaaagtacacgctgaataacatcccggcggcgtacgcggttatgctgcaaaacatggaaacgattacccgcgtgtattacggcgatctgtacacggacgacggtaattacatggaagcgaaaagcccgtactacgatacgattgttaacttgatgaagtctcgcatcaaatacgtgagcggtggccaggcgcagcgcagctactggctgccgaccgatggtaagatggataagtcggatgttgagctgtaccgtacgaacgaagtgtacacgagcgtccgttacggcaaagacattatgaccgccgatgacacgcaaggtagcaaatacagccgtaccagcggtcaggtgaccctggtcgtcaacaacccaaaactgaccttggaccaaagcgcaaagctgaacgtggttatgggcaagattcatgctaatcagaagtaccgcgcactgattgtcggtaccccgaacggtattaagaatttcaccagcgacgcagaggctattgccgcaggctatgtcaaagaaaccgatggcaatggcgtgctgaccttcggtgcaaacgacatcaagggttatgaaactttcgatatgagcggcttcgtcgctgtttgggttccggtcggtgcgagcgacgaccaagatattcgtgtggcggcgtctacggcagcaaagaaagagggtgagctgacgctgaaagcgaccgaagcctatgactcccaactgatctatgaaggctttagcaatttccagaccatcccagatggcagcgatccttctgtttataccaatcgtaagatcgcggaaaatgttgatttgttcaagagctggggtgtcacgagcttcgaaatggctccgcagttcgtttctgcggacgatggcacgtttctggacagcgtcattcaaaacggctatgcgttcgcagaccgttatgatctggccatgagcaaaaacaataagtacggtagcaaagaagatctgcgtaacgcgctgaaggcactgcacaaagcaggcattcaggcgattgcagattgggtgccagaccaaatctaccagctgcctggcaaagaagttgttactgccacccgcacggacggtgctggtcgcaaaatcagcgatgcaatcatcgatcattccctgtacgttgcgaactccaagagctccggtaaggactaccaagcgaagtacggtggcgagttcttggcggaactgaaggcgaaatacccggaaatgttcaaagtgaacatgattagcaccggcaaaccgattgatgatagcgtgaaactgaagcagtggaaagcagaatacttcaacggcaccaatgtgctggatcgcggtgtcggttatgttctgagcgatgaggcaaccggtaagtatttcaccgttaccaaagagggtaactttatcccgttgcagctgaagggtaacaagaaggtgattaccggcttttccagcgacggtaagggcattacctatttcggtactagcggtaaccaagctaaatccgcgttcgtcacttttaacggtaacacgtactacttcgacgcacgtggccacatggttaccaacggtgagtactcgccgaatggtaaagatgtgtatcgttttctgccgaacggcattatgctgagcaacgcgttctatgttgacggcaatggcaacacctacctgtacaactccaaaggccaaatgtataaaggtggctatagcaaatttgacgtcacggaaacgaaggacggtaaagagagcaaagttgtcaagttccgctactttacgaacgagggcgtgatggcgaaaggtgtcacggttgtggatggcttcactcagtactttaacgaggatggcattcaaagcaaagacgagctggtcacttacaatggcaagacctattacttcgaagcacacacgggcaatgccattaagaatacgtggcgtaatatcaagggcaaatggtaccattttgatgctaacggtgtcgcggctactggcgcacaggttatcaacggtcagcacctgtacttcaatgaagatggctctcaagtaaaaggtagcatcgtcaaaaacgctgatggtacgttcagcaagtacaaggacagctctggcgatctggtggtgaacgagtttttcacgacgggtgataacgtctggtactatgctggtgccaatggcaaaacggttactggtgcacaggtgattaatggccagcacttgttcttcaaagaggatggcagccaggtcaagggcgactttgtgaagaatagcgacggcacctactccaagtatgacgctgcgagcggcgaacgtctgaccaacgagttcttcactacgggcgacaatcattggtactatattggcgccaacggtaagaccgttaccggtgaagttaagattggtgacgacacgtatttcttcgcaaaagacggtaagcaactgaaaggtcaaatcgttaccacccgtagcggtcgtatcagctactactttggtgatagcggtaagaaggctattagcacgtgggtggagatccagccgggtgtgtttgttttcttcgacaaaaacggcctggcttacccaccggagaatatgaactga

SEQ ID NO: 20 - protein, 1340 aa, unidentified
  REGION 1..1340: note = unknown Streptococcus sp. C150
MIDGKYYYVNEDGSHKENFAITVNGQLLYFGKDGALTSSSTYSFTQGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSNYRLLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDVAALAMENKQRLALLFSLAKPIKERTPAVSPLYNNTFNTTQRDEKTDWINKDGSKAYNEDGTVKKSTIGKYNEKYGDASGNYVFIRAHDNNVQDIIAEIIKKEINEKSDGFTITDSEMKRAFEIYNKDMLSNDKKYTLNNIPAAYAVMLQNMETITRVYYGDLYTDDGNYMEAKSPYYDTIVNLMKSRIKYVSGGQAQRSYWLPTDGKMDKSDVELYRTNEVYTSVRYGKDIMTADDTQGSKYSRTSGQVTLVVNNPKLTLDQSAKLNVVMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNKKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGSIVKNADGTFSKYKDSSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 21-24 - skipped (4 entries)

SEQ ID NO: 25 - DNA, 4308 bp, Streptococcus sobrinus
  source 1..4308: mol_type = unassigned DNA
atggttgacggcaaatactactattatgatcaggatggcaacgttaagaagaatttcgcggttagcgttggtgacaagatctactactttgacgagactggtgcctacaaagacacctctaaagtggacgcggacaagtctagcagcgccgttagccaaaatgcgacgatctttgcggctaacaatcgtgcgtatagcacctctgctgagaactttgaggccgttgataactatctgacggcagatagctggtatcgtcctaaatctattctgaaagatggcaagacgtggaccgagtcgggtaaggacgacttccgtccgctgctgatggcgtggtggccggacacggagactaaacgcaattacgtgaattacatgaacctggttgtcggcatcgacaagacgtacaccgcggaaacctctcaagcagatttgaccgcagcggcggagctggtccaggcgcgtattgaacagaaaatcaccacggaacagaatacgaaatggctgcgcgaggcgatctctgctttcgtcaagacccagccgcagtggaatggtgaaagcgagaagccgtatgacgaccacctgcaaaacggtgctctgaaattcgataatcagagcgacctgaccccggacacccagagcaactatcgcctgctgaatcgcaccccgactaaccagactggcagcctggacagccgtttcacctataatgcgaacgatccgttgggtggctacgaatttctgctggctaacgacgtggataatagcaaccctgtggtgcaggcagaacaactgaactggttgcattacctgttgaattttggtagcatttacgcgaaagatgcggatgcaaacttcgattccatccgtgtggacgccgtggacaacgtcgatgcagatctgttgcagattagcagcgattacctgaaggcagcctatggcattgacaagaacaataagaacgcgaacaaccatgttagcattgttgaggcttggagcgataacgatacgccgtacctgcacgatgacggtgataacctgatgaacatggacaataagttccgcttgagcatgctgtggagcctggccaagccgctggacaagcgcagcggtctgaatcctctgattcataacagcctggtggaccgtgaggttgatgaccgtgaagtggaaacggttccgagctactcttttgcgcgtgcgcatgattccgaggtccaagacattatccgcgacattatcaaggccgaaatcaacccgaatagctttggttatagcttcacccaagaagagattgaccaggcgtttaagatctataatgaagatctgaagaaaaccgacaagaaatacacccactataatgtcccgttgagctatactttgctgctgacgaataaaggttcgattccgcgtgtgtattacggtgatatgttcaccgatgatggtcaatacatggcgaacaaaacggttaactatgatgccattgagtcgctgctgaaagcgcgcatgaagtacgttagcggcggtcaagcgatgcaaaactatcaaatcggcaatggtgagattctgaccagcgttcgttatggtaagggtgcattgaagcaatccgacaagggtgacgcgaccacgcgtacgtccggtgtgggcgtcgtgatgggcaaccagccgaactttagcctggacggcaaggtggtggcattgaacatgggtgccgctcatgcaaatcaggagtatcgtgcgctgatggtgagcaccaaggatggcgttgccacgtatgccaccgacgcggacgcaagcaaggcaggtctggtcaaacgcaccgatgaaaatggttatttgtactttctgaacgacgatctgaagggtgtggcaaacccacaagtcagcggtttcttgcaggtgtgggtcccagtgggtgcggctgacgatcaggacattcgtgttgcagcgagcgacacggctagcacggacggtaagtccctgcatcaagatgcggcaatggatagccgtgttatgtttgagggttttagcaacttccagagctttgcaaccaaagaagaagagtacaccaacgtagttattgcgaacaacgtggacaaattcgttagctggggtattaccgactttgagatggcaccgcaatatgtcagctccaccgatggccagtttctggatagcgttatccagaatggttacgcgttcaccgaccgttatgatctgggtatgagcaaagccaacaaatacggtaccgcggatcagctggttaaagcaatcaaagcgttgcacgcgaagggtctgaaggtgatggcggactgggttccagaccagatgtacacgtttccgaagcaggaagttgtcactgtcacgcgcaccgacaaatttggtaagccgattgcgggcagccaaatcaatcacagcctgtacgtgacggacaccaaatccagcggtgatgattaccaggccaaatatggtggtgcgttcctggatgagctgaaagagaaatacccggagctgttcaccaaaaagcagatctcgaccggtcaggcgatcgacccgagcgtgaagattaagcagtggagcgcgaaatactttaatggtagcaacattctgggtcgtggtgccgactacgtcctgtccgatcaagttagcaacaagtatttcaatgtggccagcgacacgctgtttctgccgtctagcctgttgggtaaggttgtcgaaagcggtattcgttacgatggcaaaggttatatctataacagcagcgcgactggcgaccaagtcaaggcgtcttttatcacggaagcaggcaatctgtactacttcggcaaagacggttacatggttactggtgcgcagaccattaacggtgcgaattacttcttcttggaaaatggtacggccctgcgtaataccatctacaccgatgcacagggcaactcccactattatgctaatgatggcaagcgttacgagaacggttaccagcagttcggcaacgattggcgttacttcaaagatggtaacatggccgtcggtctgaccacggtggatggtaacgttcagtatttcgacaaggacggtgtccaagctaaagacaagattattgtgacccgcgatggtaaggtgcgctactttgatcaacacaatggcaacgcggtcacgaatacctttatcgccgacaagaccggtcactggtactacctgggcaaagatggcgtcgcggtcaccggcgctcaaaccgtcggtaagcaaaaactgtattttgaggcgaacggtgagcaggtgaaaggcgactttgtgactagccatgaaggcaaactgtacttttatgatgttgacagcggcgacatgtggaccgataccttcatcgaggataaggccggcaactggttctacctgggtaaagacggcgcagcagttagcggtgcacagaccattcgcggtcaaaagctgtacttcaaggcgtacggtcaacaggtcaaaggtgacatcgttaaaggcaccgacggcaagatccgttactacgatgcgaaatccggcgagcaggttttcaataagacggtcaaagccgctgatggcaaaacctatgtgatcggcaacaatggtgtggcggtcgatccgagcgttgttaagggtcagacgttcaaagacgccagcggcgcactgcgtttttacaatctgaaaggtcaactggttacgggctccggttggtatgaaacggccaatcacgattgggtgtatattcagagcggtaaagcactgaccggtgagcaaaccatcaatggtcagcacctgtactttaaagaagatggccaccaagttaaaggtcagctggtcacccgtacggacggcaaagtgcgttactatgacgcaaattctggcgatcaagcgttcaacaagtccgtgacggttaacggcaaaacgtattacttcggtaatgatggtaccgcgcaaaccgcgggtaacccgaaaggccaaatcttcaaggacggcagcgttctgcgtttctatagcatggaaggccagctggtaattggcagcggctggtattccaacgcgcaaggccaatggctgtatgtgaagaatggtaaagtgttgaccggtttgcagaccgtcggttcccagcgcgtgtactttgatgagaatggcattcaagcaaaaggcaaagcggttcgcacgagcgacggcaaaattcgctacttcgacgagaacagcggtagcatgatcaccaatcaatggaagtttgtttacggtcaatactattactttggtaatgacggtgcggcaatctaccgtggttggaattaa

SEQ ID NO: 26 - protein, 1435 aa, Streptococcus sobrinus
MVDGKYYYYDQDGNVKKNFAVSVGDKIYYFDETGAYKDTSKVDADKSSSAVSQNATIFAANNRAYSTSAENFEAVDNYLTADSWYRPKSILKDGKTWTESGKDDFRPLLMAWWPDTETKRNYVNYMNLVVGIDKTYTAETSQADLTAAAELVQARIEQKITTEQNTKWLREAISAFVKTQPQWNGESEKPYDDHLQNGALKFDNQSDLTPDTQSNYRLLNRTPTNQTGSLDSRFTYNANDPLGGYEFLLANDVDNSNPVVQAEQLNWLHYLLNFGSIYAKDADANFDSIRVDAVDNVDADLLQISSDYLKAAYGIDKNNKNANNHVSIVEAWSDNDTPYLHDDGDNLMNMDNKFRLSMLWSLAKPLDKRSGLNPLIHNSLVDREVDDREVETVPSYSFARAHDSEVQDIIRDIIKAEINPNSFGYSFTQEEIDQAFKIYNEDLKKTDKKYTHYNVPLSYTLLLTNKGSIPRVYYGDMFTDDGQYMANKTVNYDAIESLLKARMKYVSGGQAMQNYQIGNGEILTSVRYGKGALKQSDKGDATTRTSGVGVVMGNQPNFSLDGKVVALNMGAAHANQEYRALMVSTKDGVATYATDADASKAGLVKRTDENGYLYFLNDDLKGVANPQVSGFLQVWVPVGAADDQDIRVAASDTASTDGKSLHQDAAMDSRVMFEGFSNFQSFATKEEEYTNVVIANNVDKFVSWGITDFEMAPQYVSSTDGQFLDSVIQNGYAFTDRYDLGMSKANKYGTADQLVKAIKALHAKGLKVMADWVPDQMYTFPKQEVVTVTRTDKFGKPIAGSQINHSLYVTDTKSSGDDYQAKYGGAFLDELKEKYPELFTKKQISTGQAIDPSVKIKQWSAKYFNGSNILGRGADYVLSDQVSNKYFNVASDTLFLPSSLLGKVVESGIRYDGKGYIYNSSATGDQVKASFITEAGNLYYFGKDGYMVTGAQTINGANYFFLENGTALRNTIYTDAQGNSHYYANDGKRYENGYQQFGNDWRYFKDGNMAVGLTTVDGNVQYFDKDGVQAKDKIIVTRDGKVRYFDQHNGNAVTNTFIADKTGHWYYLGKDGVAVTGAQTVGKQKLYFEANGEQVKGDFVTSHEGKLYFYDVDSGDMWTDTFIEDKAGNWFYLGKDGAAVSGAQTIRGQKLYFKAYGQQVKGDIVKGTDGKIRYYDAKSGEQVFNKTVKAADGKTYVIGNNGVAVDPSVVKGQTFKDASGALRFYNLKGQLVTGSGWYETANHDWVYIQSGKALTGEQTINGQHLYFKEDGHQVKGQLVTRTDGKVRYYDANSGDQAFNKSVTVNGKTYYFGNDGTAQTAGNPKGQIFKDGSVLRFYSMEGQLVIGSGWYSNAQGQWLYVKNGKVLTGLQTVGSQRVYFDENGIQAKGKAVRTSDGKIRYFDENSGSMITNQWKFVYGQYYYFGNDGAAIYRGWN

SEQ ID NO: 27 - DNA, 4023 bp, Streptococcus salivarius
  source 1..4023: mol_type = unassigned DNA
atgattgacggcaaatactactacgtaaacaaagatggctcgcacaaagagaatttcgcaattaccgtgaatggtcagttgttgtatttcggtaaggacggtgcattgacgtctagcagcacctacagctttacgcagggcaccaccaacatcgttgatggctttagcaaaaacaaccgtgcgtacgattccagcgaggcgagctttgaactgatcgacggttatctgaccgcggactcctggtatcgtccggtgagcattatcaaggacggcgttacgtggcaagccagcaccaaagaggactttcgcccgctgctgatggcctggtggccgaatgttgacacccaggtcaactacctgaattacatgtcgaaggtgtttaacctggacgcgaagtatacgagcaccgacaaacaggttgacctgaatcgcgcagccaaggacattcaggttaagattgagcaaaagattcaggccgagaagagcactcaatggctgcgtgaagcgatttcggccttcgtcaaaacccagccgcagtggaataaagaaacggagaacttctccaagggtggtggtgaggatcatctgcaaggtggtgcactgctgtacgttaacgacccgcgtaccccgtgggctaactccaactaccgcctgctgaatcgtactgcgaccaaccagaccggcacgatcgacaagagcgttctggacgaacagagcgatcctaaccacatgggcggcttcgattttctgctggcgaatgacgtcgataccagcaatccggtggtgcaggcggaacaactgaatcagatccactacctgatgaattggggttccattgttatgggcgacaaagatgcaaacttcgatggtatccgcgtggacgcggtcgataacgttgacgcagatatgctgcaactgtacaccaactactttcgtgagtattatggcgtgaacaaaagcgaggcaaacgctttggcgcacatctcggtgctggaagcgtggagcttgaatgataatcactataatgacaagactgacggtgcggccctggcgatggagaacaaacagcgtttggccctgctgtttagcttggcgaaaccgatcaaagaacgtacccctgcggtgagcccgctgtacaacaacactttcaacacgacgcagcgtgacgaaaagaccgattggattaacaaagacggtagcaaagcctataatgaggacggcaccgtcaagcagtccaccatcggcaagtacaacgagaaatacggcgacgcgtccggcaattatgtgttcattcgcgcccacgataacaacgtccaagacattattgcagagatcattaagaaagaaatcaatccgaaaagcgacggtttcaccattaccgacgccgaaatgaaaaaggcattcgaaatctacaacaaagatatgctgtcctctgataagaaatacaccctgaacaacatcccagcggcctacgcggtgatgctgcaaaacatggaaaccattactcgtgtgtattacggcgatctgtataccgacgatggccattacatggaaaccaagagcccgtactacgacaccattgtgaacctgatgaagaaccgtatcaaatacgtgtccggtggtcaagcgcaacgttcctattggctgccgaccgacggtaagatggataaaagcgatgtcgaactgtatcgcaccaacgaggtgtacaccagcgtccgttacggtaaggacatcatgactgccgatgacacccaaggtagcaagtacagccgtaccagcggtcaggtgaccctggtggtgaacaacccgaagctgtctttggataagagcgcgaagctggacgtcgaaatgggcaagatccatgcaaaccagaaataccgtgctctgatcgtgggtacgccgaacggcatcaaaaacttcacgagcgacgccgaggcaatcgcggctggctacgtgaaagaaaccgacggcaatggtgtgctgaccttcggtgcaaatgacatcaaaggttacgaaacgtttgacatgagcggtttcgttgcagtttgggttccggtaggtgcaagcgatgatcaagacatccgtgtcgccgcaagcaccgcggcaaagaaagaaggtgagctgactttgaaggcaactgaggcgtatgactctcagctgatttacgaaggtttttcgaattttcagaccattccggatggtagcgatccgagcgtttacaccaatcgtaagatcgcggaaaatgttgatttgttcaagagctggggtgtgacctctttcgaaatggcgccacagtttgtgagcgcagacgacggtacgtttctggacagcgttatccagaacggctatgcgtttgcggaccgttatgatctggcgatgtccaaaaacaataagtacggttcgaaagaagatctgcgtaacgcgttgaaggctttgcacaaggccggcatccaagccattgcggactgggttccggatcagatctaccaactgccgggcaaagaagtagtgaccgccactcgtaccgatggtgccggtcgtaagattagcgatgcaattatcgatcacagcctgtacgtcgcaaacagcaagtcgtctggcaaagactatcaagctaaatacggtggtgagttcctggccgagctgaaagcaaagtacccggaaatgtttaaagtcaacatgattagcacgggtaaaccgatcgacgactctgtcaaactgaagcaatggaaggcggagtactttaacggtacgaatgttctggaccgtggtgttggttacgtcctgagcgatgaggcgacgggcaagtactttaccgttacgaaagagggtaactttatcccactgcaattgaaaggtaacgagaaagttatcacgggcttcagctctgacggcaagggcattacctatttcggcacctcgggtaatcaagcgaaaagcgcttttgtcacgttcaatggtaatacctactattttgacgcgcgtggccacatggttaccaacggcgaatatagccctaatggtaaggatgtgtatcgtttcctgccgaatggtattatgttgagcaatgcattctacgttgacggtaacggcaatacctacctgtacaactccaagggccaaatgtacaaaggtggttatagcaaattcgacgttacggaaaccaaagatggtaaagagagcaaagtggtgaaatttcgctactttaccaatgaaggtgtgatggcaaaaggtgttaccgtggtggacggcttcactcaatacttcaacgaagatggcattcagagcaaggacgaactggtgacctacaatggtaaaacctattacttcgaagcgcataccggtaatgcgatcaaaaacacgtggcgcaatatcaagggtaagtggtatcactttgatgcgaatggcgtggcggcaacgggtgcacaggttatcaatggtcagcacctgtactttaatgaggatggttcccaggtgaagggtggcgtcgtgaagaatgcggatggtaccttcagcaagtataaagatggttccggtgacctggtggtcaatgagttcttcactactggtgataacgtgtggtactacgctggtgccaacggcaaaactgtgacgggtgcccaggtcatcaatggccaacacctgtttttcaaagaggacggtagccaggttaagggtgatttcgttaagaacagcgacggcacctactctaagtatgatgcggccagcggcgaacgcctgacgaatgagtttttcacgaccggtgacaaccactggtactatattggtgccaatggcaaaaccgttaccggcgaagtcaagatcggtgatgatacgtacttcttcgcaaaagatggcaagcagctgaagggccagatcgtgacgacccgcagcggtcgtatcagctactacttcggcgactctggtaagaaggcgattagcacctgggtggagattcagccgggtgttttcgtgtttttcgacaaaaatggcctggcatatccgccggaaaacatgaattaa

SEQ ID NO: 28 - protein, 1340 aa, Streptococcus salivarius
MIDGKYYYVNKDGSHKENFAITVNGQLLYFGKDGALTSSSTYSFTQGTTNIVDGFSKNNRAYDSSEASFELIDGYLTADSWYRPVSIIKDGVTWQASTKEDFRPLLMAWWPNVDTQVNYLNYMSKVFNLDAKYTSTDKQVDLNRAAKDIQVKIEQKIQAEKSTQWLREAISAFVKTQPQWNKETENFSKGGGEDHLQGGALLYVNDPRTPWANSNYRLLNRTATNQTGTIDKSVLDEQSDPNHMGGFDFLLANDVDTSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDDGHYMETKSPYYDTIVNLMKNRIKYVSGGQAQRSYWLPTDGKMDKSDVELYRTNEVYTSVRYGKDIMTADDTQGSKYSRTSGQVTLVVNNPKLSLDKSAKLDVEMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNEKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGGVVKNADGTFSKYKDGSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 29 - DNA, 4026 bp, Streptococcus salivarius
  source 1..4026: mol_type = unassigned DNA
atgacggacggtaaatactattatgtaaatgaggacggcagccacaaagagaatttcgcaattacggtaaacggtcaactgttgtactttggcaaggacggcgctctgacgagcagcagcacgcacagcttcacgccgggtactacgaatattgtggacggtttctcgatcaacaaccgtgcgtacgatagcagcgaagcgagctttgagctgatcaacggttacctgacggcggattcctggtatcgcccggtttctatcatcaaggatggcgtcacgtggcaggcaagcactgccgaggattttcgtccgctgttgatggcctggtggccgaacgttgatacccaggtgaactatctgaactatatgtccaaggtctttaacctggaagccaagtacaccagcaccgataaacaggctgatctgaaccgtgctgcaaaggatatccaggtcaagatcgaacagaagatccaggcggaaaagagcacgcagtggctgcgtgagactatctccgcgtttgttaaaacccagccgcaatggaacaaagagactgagaattactccaagggtggtggcgaagatcatctgcaaggcggtgcgctgttgtacgtgaacgacagccgtaccccgtgggcgaatagcaattaccgcctgctgaatcgcacggcaacgaaccagaccggtaccattaacaagtcggtgttggacgagcaatccgatccaaatcacatgggtggcttcgacttcctgctggcaaacgatgtggatctgagcaatcctgttgtgcaggccgagcagctgaatcaaatccattatctgatgaactggggcagcattgttatgggtgacaaagacgcgaattttgatggtatccgtgtggacgccgttgacaacgtgaacgctgacatgttgcagctgtacacgaactactttcgtgagtattacggcgtcaacaaaagcgaagcgcaagcgctggcgcacattagcgttctggaagcgtggagcttgaacgataaccactataacgacaaaaccgatggtgcggcactggcgatggagaataagcaacgtctggccttgctgttctctctggccaagccgatcaaagatcgtactccggcagtgagcccactgtataacaatactttcaataccacccaacgtgacttcaagacggattggattaacaaggacggtagcaccgcctacaatgaggatggcaccgcgaaacaatctaccatcggtaagtacaatgagaaatatggtgatgcaagcggtaactatgtgtttattcgtgcccatgacaataacgtccaagacattattgcggagatcattaagaaagaaatcaataagaagagcgatggttttaccatcagcgatagcgaaatgaaacaggcgttcgaaatctacaacaaagatatgctgagcagcaataagaaatacactctgaataacattccggcagcgtacgccgtgatgctgcaaaacatggagactatcacccgtgtgtattatggtgacctgtacaccgacgacggtcactatatggaaaccaagagcccgtatcatgacaccattgtgaacctgatgaaaaaccgtatcaagtacgtttctggtggccaggcccaacgctcctattggctgccgaccgacggtaaaatggacaatagcgatgtcgaactgtaccgtactagcgaggtctataccagcgttcgctacggtaaggacattatgacggcggatgacaccgagggtagcaagtactcccgcacgagcggtcaggttaccctggttgttaacaacccgaagctgactctgcatgaaagcgccaaactgaacgtcgagatgggtaagatccacgcaaaccagaaataccgtgcgctgattgtgggtaccgccgatggcatcaaaaactttacgtctgatgccgaagcgatcgcggcaggctacgtaaaagaaacggacagcaatggtgttctgaccttcggcgcaaatgatatcaaaggttacgagactttcgatatgagcggtttcgtcgcagtttgggtgccggtgggtgcgagcgatgatcaggacatccgcgtggcgccgtcgacggaagcgaagaaagaaggtgaactgacgctgaaagccacggaagcgtatgatagccagttgatttatgaaggcttctccaatttccagaccattccggatggcagcgacccgagcgtttataccaaccgcaaaattgctgagaatgttgatctgtttaagtcctggggtgtcactagcttcgaaatggctccgcagtttgtttcggcggacgacggcaccttcctggatagcgttatccagaacggttacgcctttgcggaccgttatgatttggccatgagcaagaacaacaagtacggttctaaagaggatctgcgcgacgcactgaaagcgctgcacaaagctggcattcaggcaatcgcggactgggtcccagaccaaatctaccaactgccaggcaaagaagtggttacggcgacgcgcacggacggtgcgggtcgcaagatcgcggacgccatcattgatcatagcctgtatgttgctaactccaagagctccggtcgcgattaccaagcgcagtatggtggcgagtttctggcagagctgaaagcgaagtacccgaaaatgttcacggaaaacatgattagcacgggtaagccgatcgatgacagcgtcaaactgaagcaatggaaagccaagtatttcaatggtacgaatgtgctggaccgtggtgtcggttacgtcctgtccgacgaggcgaccggcaaatacttcaccgttaccaaagagggtaacttcattccgctgcaactgaccggcaatgaaaaagcggtgaccggtttcagcaacgacggcaagggtatcacctactttggtacgagcggtaatcaggccaagagcgcgttcgtcacctttaacggcaatacgtactatttcgacgcgcgtggccacatggtcacgaacggcgagtatagcccgaacggcaaagatgtctaccgttttctgccaaatggtattatgttgtcgaacgcgttttatgtcgacgcaaacggtaatacgtacttgtacaactacaagggccagatgtacaaaggtggttatacgaaatttgatgtcaccgaaactgataaagatggtaatgagagcaaggtggtcaagtttcgttatttcaccaatgagggcgtcatggctaagggtctgaccgtcattgacggtagcacccagtactttggtgaggatggttttcaaacgaaggacaagctggcgacctataaaggtaagacttattacttcgaggcacacacgggcaatgcgatcaaaaacacctggcgtaacatcgacggtaagtggtatcacttcgatgagaatggcgttgccgcgaccggtgcacaagtgattaacggtcaaaaactgtatttcaacgaggatggctcgcaagtgaagggcggtgttgttaagaacgccgacggtacctacagcaaatacaaagagggcagcggtgagctggttaccaacgagtttttcacgaccgacggtaatgtgtggtactatgctggtgcggatggcaagactgtgaccggtgctcaggtcattaatggtcagcacctgtactttaaagaagatggcagccaggtgaaaggtggtgtggtgaaaaacgcggacggtacgtacagcaagtatgacgccgccaccggtgaacgcttgaccaatgagttctttaccacgggcgataacaattggtactatattggttctaatggtaagaccgtaaccggtgaagtcaaaatcggtgcggacacctattactttgccaaagatggcaaacaggtcaagggccaaaccgtcaccgcaggcaatggccgcatctcctattactacggcgattctggtaagaaagcaatcagcacgtggatcgaaattcaaccgggtatctatgtctattttgataagacgggcatcgcgtacccaccgcgtgtgctgaattaa

SEQ ID NO: 30 - protein, 1341 aa, Streptococcus salivarius
MTDGKYYYVNEDGSHKENFAITVNGQLLYFGKDGALTSSSTHSFTPGTTNIVDGFSINNRAYDSSEASFELINGYLTADSWYRPVSIIKDGVTWQASTAEDFRPLLMAWWPNVDTQVNYLNYMSKVFNLEAKYTSTDKQADLNRAAKDIQVKIEQKIQAEKSTQWLRETISAFVKTQPQWNKETENYSKGGGEDHLQGGALLYVNDSRTPWANSNYRLLNRTATNQTGTINKSVLDEQSDPNHMGGFDFLLANDVDLSNPVVQAEQLNQIHYLMNWGSIVMGDKDANFDGIRVDAVDNVNADMLQLYTNYFREYYGVNKSEAQALAHISVLEAWSLNDNHYNDKTDGAALAMENKQRLALLFSLAKPIKDRTPAVSPLYNNTFNTTQRDFKTDWINKDGSTAYNEDGTAKQSTIGKYNEKYGDASGNYVFIRAHDNNVQDIIAEIIKKEINKKSDGFTISDSEMKQAFEIYNKDMLSSNKKYTLNNIPAAYAVMLQNMETITRVYYGDLYTDDGHYMETKSPYHDTIVNLMKNRIKYVSGGQAQRSYWLPTDGKMDNSDVELYRTSEVYTSVRYGKDIMTADDTEGSKYSRTSGQVTLVVNNPKLTLHESAKLNVEMGKIHANQKYRALIVGTADGIKNFTSDAEAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGRDYQAQYGGEFLAELKAKYPKMFTENMISTGKPIDDSVKLKQWKAKYFNGTNVLDRGVGYVLSDEATGKYFTVTKEGNFIPLQLTGNEKAVTGFSNDGKGITYFGTSGNQAKSAFVTFNGNTYYFDARGHMVTNGEYSPNGKDVYRFLPNGIMLSNAFYVDANGNTYLYNYKGQMYKGGYTKFDVTETDKDGNESKVVKFRYFTNEGVMAKGLTVIDGSTQYFGEDGFQTKDKLATYKGKTYYFEAHTGNAIKNTWRNIDGKWYHFDENGVAATGAQVINGQKLYFNEDGSQVKGGVVKNADGTYSKYKEGSGELVTNEFFTTDGNVWYYAGADGKTVTGAQVINGQHLYFKEDGSQVKGGVVKNADGTYSKYDAATGERLTNEFFTTGDNNWYYIGSNGKTVTGEVKIGADTYYFAKDGKQVKGQTVTAGNGRISYYYGDSGKKAISTWIEIQPGIYVYFDKTGIAYPPRVLN

SEQ ID NO: 31-32 - skipped (2 entries)

SEQ ID NO: 33 - DNA, 4026 bp, Streptococcus salivarius
  source 1..4026: mol_type = unassigned DNA
atgatcgacggcaaatactactatgtaaacgaggacggcagccacaaagagaatttcgcgattacggtaaacggtcagctgctgtactttggtaaggacggtgctctgacgagcagctccacgtacagctttaccccgggtacgaccaatattgtcgatggcttcagcattaacaaccgtgcgtatgacagcagcgaggcatcctttgagctgatcgatggttatttgaccgcggatagctggtatcgtccggcgagcatcattaaggacggcgttacgtggcaggcctcgaccgcagaagattttcgtccgctgctgatggcttggtggccgaatgttgacacccaggtgaattatctgaattacatgtccaaggttttcaacctggatgcaaagtacaccagcaccgacaagcaggaaaccctgaacgtggctgcgaaagatatccaagtcaagattgagcaaaagattcaggcagagaaatctacccagtggctgcgtgaaacgattagcgcgtttgttaaaactcagccgcaatggaataaagaaacggaaaactattccaagggtggtggcgaggaccatctgcaaggcggtgccctgttgtacgttaacgattcgcgcaccccgtgggcgaactcgaactatcgcttgctgaaccataccgctaccaatcaaaaaggcactattgacaaatctgtcctggacgagcagagcgacccgaaccacatgggcggtttcgattttctgctggcgaacgacgtcgacctgagcaacccggtggtgcaggccgaacaactgaaccagattcactacctgatgaattggggtagcatcgtgatgggtgataaagatgcgaactttgacggcattcgtgtcgatgcggtcgataacgtggacgccgacatgttgcagctgtacacgaactactttcgtgagtactacggcgttaacaagagcgaagcaaatgccctggcgcatatcagcgttctggaagcgtggagcctgaatgacaatcactataacgataagacggacggtgcggccctggcaatggagaataaacaacgtctggcgctgctgttcagcctggcgaaaccgatcaaagagcgtacgccggctgtgagcccactgtataacaacaccttcaatactacgcagcgtgacgagaaaacggactggattaacaaagacggtagcaaagcgtataacgaggatggtaccgtcaagcaatcgaccattggtaagtacaatgagaagtatggcgacgcaagcggtaattacgtgttcattcgtgcccacgacaacaatgttcaagacatcatcgccgaaatcatcaagaaagagatcaaccctaagagcgacggtttcaccatcaccgacgcagagatgaagaaggcctttgaaatctacaacaaggacatgttgagcagcgataagaagtatactctgaacaacattccggctgcgtacgcggtgatgttgcagaatatggaaaccatcacgcgtgtttactatggtgatctgtataccgataatggcaactacatggaaacgaaaagcccgtactatgacaccattgttaatctgatgaagaatcgcatcaagtatgtgtctggcggtcaagcgcagcgttcttactggctgccgaccgatggtaagatggacaatagcgatgtggaactgtaccgcaccaacgaggtatacgcttctgtgcgctatggtaaagacattatgaccgccgatgataccgagggttccaagtactcccgtacgagcggccaagttaccttggtggcaaacaacccgaaattgaccctggaccaaagcgcgaaactgaaagtggagatgggtaagatccacgcaaatcaaaagtaccgtgcactgattgtcggtaccgccgacggtatcaagaatttcaccagcgatgcggatgcgattgcagcaggctatgttaaagagactgatagcaatggtgtgctgacgtttggtgcgaacgacattaaaggctatgaaacgtttgacatgagcggtttcgttgcggtgtgggtgcctgtgggtgctagcgatgatcaggatatccgtgtcgcgccgagcaccgaggcaaagaaagaaggtgagctgacgttgaaagcgaccgaggcctatgacagccagttgatttacgaaggtttcagcaatttccaaaccattccagacggttccgatccgagcgtctacaccaatcgcaaaatcgcggaaaacgttgatctgttcaaaagctggggtgtgaccagcttcgaaatggcaccgcaattcgttagcgcggacgatggtacgttcttggacagcgttatccaaaatggctatgcgttcgccgatcgttatgacttggcgatgagcaaaaacaacaaatacggcagcaaagaggatctgcgcgacgccctgaaagcgctgcataaagcgggtattcaagccatcgctgactgggttccggaccagatctaccagctgccgggtaaagaagtcgttaccgcgacccgcaccgatggcgctggccgtaagatcgcggatgcaattatcgatcatagcttgtatgtggccaatactaaaagctccggtaaggattaccaggcgaaatatggtggtgaatttctggctgagctgaaggccaaatacccggagatgttcaaggtcaacatgattagcaccggcaaacctattgatgactctgtcaaattgaaacaatggaaggcagagtatttcaatggcactaacgtcctggaacgtggtgttggttacgtgctgagcgacgaggcgaccggtaaatacttcaccgttacgaaggacggcaatttcatcccgctgcaactgaccggtaatgagaaggttgtgacgggtttttctaatgacggtaagggcattacctacttcggtacctcgggtacccaggcaaagagcgcattcgtgacgtttaacggtaacacctactactttgatgcacgcggccacatggtgacgaacggcgagtacagcccgaacggcaaggatgtttatcgcttcctgccgaatggcatcatgctgtccaatgcgttttacgtcgatgcaaatggtaatacttacctgtacaacagcaagggtcagatgtataagggcggttataccaagttcgacgttactgaaacggacaaggacggtaaagagagcaaagtagtgaagtttcgttatttcacgaacgaaggcgtcatggcgaaaggtgtcaccgttattgatggctttacccagtatttcggtgaagatggctttcaagcgaaggacaagctggtgacctttaagggcaaaacctactattttgacgcgcacacgggcaacgccatcaagaacacctggcgtaatatcgacggtaagtggtatcattttgatgcgaacggtgtggcggcgaccggcgcacaggtcattaatggtcaaaaactgtactttaatgaggacggtagccaagtcaaaggtggcgtcgtcaagaatgcagatggcacctatagcaaatacaaagagggctccggtgagctggttaccaacgagttctttaccacggatggtaacgtctggtactatgctggtgcgaatggcaagaccgttaccggtgcacaggttatcaacggccagcacctgtacttcaatgcggatggctctcaagtgaagggcggtgtcgtcaaaaacgcggacggtacgtactccaaatacgatgccgcgaccggtgaacgtctgaccaatgagtttttcacgactggtgacaacaattggtactacatcggcgccaacggtaagacggttacgggcgaagtgaaaattggcgacgatacgtactacttcgcaaaagatggtaaacaggtgaaaggtcagacggtttccgctggtaatggccgcatcagctactattacggtgactctggtaaacgtgcggttagcacgtgggttgaaattcaaccgggcgtgtatgtctattttgataagaatggcctggcatatccaccgcgcgttttgaattaa

SEQ ID NO: 34 - protein, 1341 aa, Streptococcus salivarius
MIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYTSTDKQETLNVAAKDIQVKIEQKIQAEKSTQWLRETISAFVKTQPQWNKETENYSKGGGEDHLQGGALLYVNDSRTPWANSNYRLLNHTATNQKGTIDKSVLDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDNGNYMETKSPYYDTIVNLMKNRIKYVSGGQAQRSYWLPTDGKMDNSDVELYRTNEVYASVRYGKDIMTADDTEGSKYSRTSGQVTLVANNPKLTLDQSAKLKVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKNTWRNIDGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDAATGERLTNEFFTTGDNNWYYIGANGKTVTGEVKIGDDTYYFAKDGKQVKGQTVSAGNGRISYYYGDSGKRAVSTWVEIQPGVYVYFDKNGLAYPPRVLN

SEQ ID NO: 35-58 - skipped (24 entries)

SEQ ID NO: 59 - protein, 1242 aa, Streptococcus sp.
MINGKEYYVEDDGTVRKNYVLERNGGSQYFNAETGELSNQKDYRFDKNGGTGSAADSTTNTNVTVNGDKNAFYGTTEKDIELVDGYFTANTWYRPKEILKDGKEWTASTENDKRPLLTVWWPSKAIQASYLNYMREEGLGTNQTFTSYSSQTQMDQAALEVQKRIEERIAREGNTDWLRTTIKNFVKTQPGWNSTSENLDNSDHLQGGALLYNNSNRTSYANSDYRLLNRTPTQQDGTRRYFKDNSSGGFEFLLANDIDNSNPAVQAEQLNWLHYIMNIGSLTGGSEDENFDGVRVDAVDNVNADLLQIASDYFKAKYGVEKSEEEAIKHLSILEAWSHNDAYYNEDTKGAQLPMDDPLRLAMVFSFLRPIGNRSGLEPLITNSLNDRSESKKNTKRMANYTFVRAHDSEVQSVIGQIIKNEINPQSTGNTFTLDEMKKAFKIYNADMRSANKRYTQYNIPSAYAFMLTNKDTVPRVYYGDLYTDDGQYMAQKSPYHDAISTLLQARIRYAAGGQDMKMSYVGSGNTNGWDASGVLTSVRYGKGANNASDAGTAETRNQGMAVILSNQPALRLNSNLTINMGAAHRNQAYRPLLLTTSNGVASYLNDGDANGIVKYTDANGYLTFNPGEISGVRNAQVDGYLAVWVPLGASENQDVRVAASKSKNSSGLVYDSSAALDSQVIYEGFSNFQDFVQDPSQYTNKKIAENANLFKSWGITSFEFAPQYVSSDDGTFLDSVIQNGYAFSDRYDIGMSKDNKYGSLADLKAALKSLHAVGISAIADWVPDQIYNLPGDEVVTATRVNNYGETKDGAIIDHSLYVAKTRTFGNDYQGKYGGAYLDELKRLYPQFFDRVQISTGKRLTTDEKITKWSAKYMNGTNILDRGSEYVLKNGLSGYYGTNGGKVSLPKVVGSNQSTNNNNQNGDGSGRFEKSWGSVYYRYNDGQRARNAFIKDNDGNVYYFDNTGRMAIGEKTIDGKQYFFLANGVQLRDGYRQNRRGQVFYYDENGIMSQTGKPSPKPEPKPDNNTFSRNQFIQIGNNVWAYYDGNGKRVIGRQNINGQELFFDNNGVQVKGRTAQVDGVTRYFDANSGEMARNRFAEVEPGVWAYFNNDGAAVTGSQNINGQTLYFDQNGHQVKGALVTVDGNLRYYDANSGDLYRNRFQEVNGSWYYFDGNGNAVKGMVNINGQNLLFDNDGKQVKGHLVRVNGVIRYYDPNSGEMAVNRWVEISSGWWVYFDGEGRGQI

SEQ ID NO: 60 - protein, 1518 aa, Streptococcus salivarius
MENKIHYKLHKVKKQWVTIAVASVALATVLGGLSVTTSSVSADETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 61 - protein, 1528 aa, Streptococcus salivarius
MTNKITGKIIMENKVHYKLHKVKKQWVTIAVASAALATVVGGLSATTSSVSADETQDKIVTQPNLDTTADLVTSTEATKEVDKRTNTKEADVLTPAKETNAVETATTTNTQATAEAATTATTSDVAVAAVPNKEAVVTTDAPAVTTEKAEEQPATVKAEVVNTEVKAPQAALKDSEVEAALSLKNIKYTDGKYYYVNEDGSHKENFAITVNGQLLYFGKDGALTSSSTHSFTPGTTNIVDGFSINNRAYDSSEASFELINGYLTADSWYRPVSIIKDGVTWQASTAEDFRPLLMAWWPNVDTQVNYLNYMSKVFNLEAKYTSTDKQADLNRAAKDIQVKIEQKIQAEKSTQWLRETISAFVKTQPQWNKETENYSKGGGEDHLQGGALLYVNDSRTPWANSNYRLLNRTATNQTGTINKSVLDEQSDPNHMGGFDFLLANDVDLSNPVVQAEQLNQIHYLMNWGSIVMGDKDANFDGIRVDAVDNVNADMLQLYTNYFREYYGVNKSEAQALAHISVLEAWSLNDNHYNDKTDGAALAMENKQRLALLFSLAKPIKDRTPAVSPLYNNTFNTTQRDFKTDWINKDGSTAYNEDGTAKQSTIGKYNEKYGDASGNYVFIRAHDNNVQDIIAEIIKKEINKKSDGFTISDSEMKQAFEIYNKDMLSSNKKYTLNNIPAAYAVMLQNMETITRVYYGDLYTDDGHYMETKSPYHDTIVNLMKNRIKYVSGGQAQRSYWLPTDGKMDNSDVELYRTSEVYTSVRYGKDIMTADDTEGSKYSRTSGQVTLVVNNPKLTLHESAKLNVEMGKIHANQKYRALIVGTADGIKNFTSDAEAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGRDYQAQYGGEFLAELKAKYPKMFTENMISTGKPIDDSVKLKQWKAKYFNGTNVLDRGVGYVLSDEATGKYFTVTKEGNFIPLQLTGNEKAVTGFSNDGKGITYFGTSGNQAKSAFVTFNGNTYYFDARGHMVTNGEYSPNGKDVYRFLPNGIMLSNAFYVDANGNTYLYNYKGQMYKGGYTKFDVTETDKDGNESKVVKFRYFTNEGVMAKGLTVIDGSTQYFGEDGFQTKDKLATYKGKTYYFEAHTGNAIKNTWRNIDGKWYHFDENGVAATGAQVINGQKLYFNEDGSQVKGGVVKNADGTYSKYKEGSGELVTNEFFTTDGNVWYYAGADGKTVTGAQVINGQHLYFKEDGSQVKGGVVKNADGTYSKYDAATGERLTNEFFTTGDNNWYYIGSNGKTVTGEVKIGADTYYFAKDGKQVKGQTVTAGNGRISYYYGDSGKKAISTWIEIQPGIYVYFDKTGIAYPPRVLN

SEQ ID NO: 62 - protein, 1518 aa, Streptococcus salivarius
MENKIHYKLHKVKKQWVTIAVASVALATVLGGLSVTTSSVSADETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLTLDQSAKLNVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKDTWRNINGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDASTGERLTNEFFTTGDNNWYYIGANGKSVTGEVKIGDDTYFFAKDGKQVKGQTVSAGNGRISYYYGDSGKRAVSTWIEIQPGVYVYFDKNGIAYPPRVLN

SEQ ID NO: 63 - protein, 1431 aa, Streptococcus salivarius
MTKETNTVDAATTTNTQAAADAATKTADAAVTALPNKEAVVTTDAPAVTTEKAAEQPATVKSEVVNTEVKAPEAALKDSEVEAALSLKNIKNIDGKYYYVNKDGSHKENFAITVNGQLLYFGKDGALTSSSTYSFTQGTTNIVDGFSKNNRAYDSSEASFELIDGYLTADSWYRPVSIIKDGVTWQASTKEDFRPLLMAWWPNVDTQVNYLNYMSKVFNLDAKYTSTDKQVDLNRAAKDIQVKIEQKIQAEKSTQWLREAISAFVKTQPQWNKETENFSKGGGEDHLQGGALLYVNDPRTPWANSNYRLLNRTATNQTGTIDKSVLDEQSDPNHMGGFDFLLANDVDTSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDDGHYMETKSPYYDTIVNLMKNRIKYVSGGQAQRSYWLPTDGKMDKSDVELYRTNEVYTSVRYGKDIMTADDTQGSKYSRTSGQVTLVVNNPKLSLDKSAKLDVEMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNEKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGGVVKNADGTFSKYKDGSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 64 - protein, 1532 aa, unidentified
  REGION 1..1532: note = unknown Streptococcus sp. C150
MENKVHYKLHKVKKQWVTIAVASAALATVVGGLSATTSSVSADETQDKTVTQPNSDTTADLVTSTEATKEVDKRTNTKEADVLTPAKETNTVETAATTNTQATAEAAKTATTTNTQATAEVAKTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTQGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSNYRLLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDVAALAMENKQRLALLFSLAKPIKERTPAVSPLYNNTFNTTQRDEKTDWINKDGSKAYNEDGTVKKSTIGKYNEKYGDASGNYVFIRAHDNNVQDIIAEIIKKEINEKSDGFTITDSEMKRAFEIYNKDMLSNDKKYTLNNIPAAYAVMLQNMETITRVYYGDLYTDDGNYMEAKSPYYDTIVNLMKSRIKYVSGGQAQRSYWLPTDGKMDKSDVELYRTNEVYTSVRYGKDIMTADDTQGSKYSRTSGQVTLVVNNPKLTLDQSAKLNVVMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNKKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGSIVKNADGTFSKYKDSSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 65 - protein, 1341 aa, Streptococcus salivarius
MIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 66 - protein, 1614 aa, Lactobacillus animalis
DETSSSNETQTEQTLNTDESTDTTTDVSNEAKATEAQLTTQDADMASSEEKTTNVEKEVTTAETNKDTTVKNVESSEQNTTTVADKNAVDSTAQVNTAEKENKYTQENVNGNWYLKDEQGNYLTGFQEIKDQNKTVYYNPDSKQMVYGQQNINGNWYLFDTFNGAMQTGLQYIRDQKKLAYYNEQGQMQYGTVEIDGQKYQADTFNGAIKGKGQTKIADNWYLFNNAGQVVDGWQWINDQGKTVYYSTKTAQMVHGQQNINGHWYLFDKTTGAMQRGFQNLKAYGDDKTVYYNQDGWMLYGQQKIDNKWYNFDTFNGAMKTGFVKIPEQNKTVYYAPNGQMQYGWQWVDNATRYFDTFNGAMATGQKLITGHWYLFDNNGAMQRGFQNLKNYGDNKTVYYNQDGWMLYGWQWVNNATRYFDTFNGAMTTGQKKINDHWYLFDKDGAMQRGIQYIPEENKLVYYNQDGWMLYGKQNINGVDHNFNTFNGALEAKGQVKVGNNWYLFNNSGTIQTGFQDLKAYGQDKVVYYDPKTAAMVYGYQNIDGNWYLFSRANGSMQRGLQNVNGVDLLFDEKTGALLTGVQNIKGNNYFVDKRSGNIKKNLVVLGADNKWMYFDAKTGKGTNTLEDQYKKGVVSGNVEFITNNAAYSFDGNSFENINGFLTADSWYRPKSILKDGSTWTATTETDLRPLLMTWWPNEQIKANYLNYMKDKGFINNSGTYNAESDPNYMDFAAQEAQRNIERKITKENDTTWLRDLITDFIKTQDIWNEQSEGVSTEGLQKFQGGFLKYVNSELTPYANSEWRKLGYQPTMLTQNNVGAEFLLANDIDNSNPVVQAEQLNWLHFLMNFGTITANDPSANFDGIRIDAVDNVDASLLSIAGDYFKAAYKVGQNDATANKHISILEDWNDKDPEYVNSIGNPQLTMDDYIVQQLKFSLGQAPDKVDRMQRFKEWYLVDRSKDNTENTAIPNYSFVRAHDASVQEDILQLIQDTTGKPWGVYTNEELQQGLKDYMADQKLTNKKYNRYNIPSSYAILLTNKDTIPRVYYGDLYSDAGKYMAEKSIYFDAIDNLLKTRTKYVAGGQTLDVDGHDILTSVRFGKGALNVTDKGTSETRTQGMGLIISNNNSLKLNDGEKVVLHMGAAHKNQAYRAVMLSSANGLINYTSDANAPVVYTNNDGDLIFTNKDVVTNGKVQANTAIKGVMNPYVSGYLAMWVPVGASATQDARTAASTKTTTDGSVFHSNAALDSNLIYEGFSNFQAFPENASENANAIIAQNVDLFNSWGVTSFQLAPQYVSSHDGSFLDSIIDNGYAFTDRYDLAMSKNNKYGSYQDLVNVLKALHAGGIQVIADWVPDQIYSLPGKEVVSVVRSDEFGNKVDGTQIDNTLYVVNTIGGGQYQKEYGGRYLEELKQKYPELFKTKQPSTGVTIDPSEKITEWSAKYLNGTNILHRGAEFVLRDGATYFRVAETSEVFLPSQLRGKITKNGFWKNDAGKVNYYNSEGEIMKNAFVKDGKNNWYYFDNDGNMVTNTALTIDSDAQVADYYFLNNGISLRDGFVQLANGDIYYYDVNGRKLKNGKVTVNNVEYTTDKNGKVVGENVLKKLDEIITTGKTTLI

SEQ ID NO: 67 - protein, 1557 aa, Streptococcus salivarius
EETNNSNGSPSTTTVGENTNPVVEKEVGTTTEVANTSNATTTERAEVTADKPAETTVQPNSGTTTSDRAVAVEVEAKPETTAKPEVATKPETATTSEVAANAGVAAPTTEKSKELSEAEIKAAVSLDNIKKEKDGKYYYLLEDGSHKKNFAITVNGQVLYFDENGALSSTSTYSFTQETTNLVTDFTKNNAAYDSTKASFELVDGYLTADSWYRPKEILEAGTTWKASTEKDFRPLLMSWWPDKDTQVAYLNYMTKALSNGEETKDVFTIENSQASLNAAAQILQRKIEVKIAANKSTDWLRQSIEAFVKDQDKWNINSESPGKEHFQKGALLFVNSDSTKWANSDYRKLNQTATSYIKNHKIVNGSDGGYEFLLSNDIDNSNPVVQAEMLNQLYYFMNWGQIVFGDKDKDAHFDGIRVDAVDNVSVDMLQLVSSYMKAAYKVNESEARALANISILEAWSHNDPYYVNEHNTAALSMDNGLRLSIVHGLTRPVTNKGTGARNASMKDLINGGYFGLSNRAEVTSYDQLGFATYLFVRAHDSEVQTVIADIISKKIDPTTDGFTFTLDQLKQAFDIYNADMLKVDKEYTHSNIPAAYALMLQTMGAATRVYYGDLYTDNGQYMAKKSPYFDQITTLLKARPKYVAGGQTSYIHNLAGDGVSSAKDNKEVLVSVRYGQDLMSKTDTEGGKYGRNSGMLTLIANNPDLKLADGETITVNMGAAHKNQAYRPLLLGTDKGIVSSLNDSDTKVVKYTDAQGNLVFTADEIKGFKTVDMSGYLSVWVPVGATDDQNVLAKPSTKAYKEGDKVYSSSAALEAQVIYEGFSNFQDFVKEDSQYTNKLIAANADLFKSWGITSFEIAPQYVSSKDGTFLDSIIENGYAFTDRYDFAMSKNNKYGSKEDLRDALKALHKQGIQVIADWVPDQLYTLPGKEVVTATRTDTHGKVLDDTSLVNKLYVTNTKSSGNDFQAQYGGAFLDKLQKLYPEIFKEVMEASGKTIDPSVKIKQWEAKYFNGTNIQKRGSDYVLSDGKLYFTVNDKGTFLPAALTGDTKAKTGFAYDGTGVTYYTTSGTQAKSQFVTYNGKQYYFNDKGYLVTGEQTIDGSNYFFLPNGVMFTDGVRKNAKGQSLVYGKSGKLTTQTGWKEVTVKDDSGKEEKFYQYFFKGGIMATGLTEVEGKEKYFYDNGYQAKGVFVPTKDGHLMFFCGDSGERKYSGFFEQDGNWYYANDKGYVATGFTKVGKQNLYFNEKGVQVKNRFFQVGDATYYANNEGDVLRGAQTINGDELYFDESGKQVKGEFVNNPDGTTSYYDAITGVKLVDTSLVVDGQTFNVDAKGVVTKAHTPGFYTTGDNNWFYADSYGRNVTGAQVINGQHLYFDANGRQVKGGFVTNTDGSRSFYHWNTGDKLVSTFFTTGHDRWYYADDRGNVVTGAQVINGQKLFFDTDGKQVKGAFATNANGSRSYYHWNTGNKLVSTFFTSGDNNWYYADAKGEVVVGEQTINGQHLYFDQTGKQVKGATATNPDGSISYYDVHTGEKAINRWVKIPSGQWVYFNAQGKGYVS

SEQ ID NO: 68 - protein, 1552 aa, Lactobacillus animalis
EETNNSNGSPSTTTVGENTNPVVEKEVGTTTEVANTSNATTTERAEVTADKPAETTVQPNSGTTTSDRAVAVEVEAKPETTAKPEVATKPETATTSEVAANAGVAAPTTEKSKELSEAEIKAAVSLDNIKKEKDGKYYYLLEDGSHKKNFAITVNGQVLYFDENGALSSTSTYSFTQETTNLVTDFTKNNAAYDSTKASFELVDGYLTADSWYRPKEILEAGTTWKASTEKDFRPLLMSWWPDKDTQVAYLNYMTKALSNGEETKDVFTIENSQASLNAAAQILQRKIEVKIAANKSTDWLRQSIEAFVKDQDKWNINSESPGKEHFQKGALLFVNSDSTKWANSDYRKLNQTATSYIKNHKIVNGSDGGYEFLLSNDIDNSNPVVQAEMLNQLYYFMNWGQIVFGDKDKDAHFDGIRVDAVDNVSVDMLQLVSSYMKAAYKVNESEARALANISILEAWSHNDPYYVNEHNTAALSMDNGLRLSIVHGLTRPVTNKGTGARNASMKDLINGGYFGLSNRAEVTSYDQLGFATYLFVRAHDSEVQTVIADIISKKIDPTTDGFTFTLDQLKQAFDIYNADMLKVDKEYTHSNIPAAYALMLQTMGAATRVYYGDLYTDNGQYMAKKSPYFDQITTLLKARPKYVAGGQTSYIHNLAGDGVSSAKDNKEVLVSVRYGQDLMSKTDTEGGKYGRNSGMLTLIANNPDLKLADGETITVNMGAAHKNQAYRPLLLGTDKGIVSSLNDSDTKVVKYTDAQGNLVFTADEIKGFKTVDMSGYLSVWVPVGATDDQNVLAKPSTKAYKEGDKVYSSSAALEAQVIYEGFSNFQDFVKEDSQYTNKLIAANADLFKSWGITSFEIAPQYVSSKDGTFLDSIIENGYAFTDRYDFAMSKNNKYGSKEDLRDALKALHKQGIQVIADWVPDQLYTLPGKEVVTATRTDTHGKVLDDTSLVNKLYVTNTKSSGNDFQAQYGGAFLDKLQKLYPEIFKEVMEASGKTIDPSVKIKQWEAKYFNGTNIQKRGSDYVLSDGKLYFTVNDKGTFLPAALTGDTKAKTGFAYDGTGVTYYTTSGTQAKSQFVTYNGKQYYFNDKGYLVTGEQTIDGSNYFFLPNGVMFTDGVRKNAKGQSLVYGKSGKLTTQTGWKEVTVKDDSGKEEKFYQYFFKGGIMATGLTEVEGKEKYFYDNGYQAKGVFVPTKDGHLMFFCGDSGERKYSGFFEQDGNWYYANDKGYVATGFTKVGKQNLYFNEKGVQVKNRFFQVGDATYYANNEGDVLRGAQTINGDELYFDESGKQVKGEFVNNPDGTTSYYDAITGVKLVDTSLVVDGQTFNVDAKGVVTKAHTPGFYTTGDNNWFYADSYGRNVTGAQVINGQHLYFDANGRQVKGGFVTNTDGSRSFYHWNTGDKLVSTFFTTGHDRWYYADDRGNVVTGAQVINGQKLFFDTDGKQVKGAFATNANGSRSYYHWNTGNKLVSTFFTSGDNNWYYADAKGEVVVNGQHLYFDQTGKQVKGATATNPDGSISYYDVHTGEKAINRWVKIPSGQWVYFNAQGKGYVS

SEQ ID NO: 69 - protein, 1302 aa, Streptococcus criceti
ADATDGNGGNTQVAHLIPKEPTDYKFDTPSGILTGLNFANAQTSPAGDNAGANQPAGGIEPQTAENAATDGQAVPQTSDQPGHLENVDGKTYYVDANGQRLKNYSTVIDGKTYYFDAQTGQAQAETPQINQNDNQVAPDTYAANNQAFTNDVSSFETVDNYVTADSWYRPRKILKNGESWQASAESDMRPILMTWWPDAATKAAYANYWVKEGLISGSYSPNSANLETAVQTIQAAIEKKIASEGSTAWLRDKMSQFVKSQNQWSLASENPTVYPNQDHLQGGALLFSNNEATAHANSDWRLLNRNPTFQTGKQKYFTTNYAGYELLLANDVDNSNPIVQAEQLNHFHYLMNWGEIVMGDKNANFDGVRVDAVDNVNADLLQIQRDYYKAKYGVDQNEKNAIDHLSILEAWSGNDNDYVKDQNNFSLSIDNSQRSYMLAAFAYPASQRGNDYISLLPKVGLKDRRYAKNGNPVPNYVFIRAHDSEVQTRIAKIIRERLGKTNADGLTNITLDDLNKAFDIYNQDMKAVDKQYYPNNLPMAYAWMLQNKDTVTRVYYGDMYTDDGQYMETKTPFHDAIETLLKARIKYVAGGQTAGYVQGWGSGILTSVRYGKGADTAIDAGTAETRTSGMAVLINNKPNFQSYNGLTLDMGAAHKNQAYRPLLLSTKDGIATYLNDSDVSSNQYKYTDGQGRLNFSASELRSVANVQVSGMIQVWVPVGAADNQDVRVAPNTNRNNSSNIYTQSDALDSQVIYEGFSNFQAFAKTPEQYTNAVIAKNADLFKSWGITQFEMAPQYVSSEDGTFLDSVVLNGYAFSDRYDLAMSKNNKYGSKEDLANAIKGLHNAGIKVLSDWVPDQMYNLPGKEVVTATRVDQYGRPKAGATINRTPYVVNTKTYGDYQEQYGGKFLDELQKLYPSLFTTKQISTGKPIDPSVKITNWSAKYFNGSNILGRGAKYVLSDNNKYLNLGAGQFFLPTNLNNTYGQPQAPANGFISKNGGIHYIDNNGQEVKNQFKEIAGSWYYFDANGKMATGQTKIGNTTYLFMPNGKQLKEGVWYDGKKAYYYDDNGRTWTNKGFVEFKVNGQDKWRYFNGDGSIAVGLVSLDNRTLYFDAYGYQVKGQTLTINGKTYSFDANEGDLITGNTPSPEPNNQGAWEALGDNQWGYRKDGKLLTGSQTIAGQKVFFQPNGVQVKGGTAKDEAGVLRFYDRDQGHLAGKGWYSTADNNWVYVDDAGRVVTGLQKIGSQTLYFDDNGIQAKGKAIWDKDGNLRYFAAGSGDMITNRWYNIGDNQWYWFNNQGIASRW

SEQ ID NO: 70 - protein, 1107 aa, Leuconostoc mesenteroides
VKGDKRTIDGVLYTFDKESGERKGLDSISVLPTNGQYTTDKAQNWYYQVDGENVKGLYTNNDGQLRYFDLTTGVQTKGNFVTIGNDTYYFTKEQGDGQIVSEVVSGHYGTVQLSDNSSAWVYRGANDQILKGLQNINGRLQYFDLTTGAQLKGGAANYDGNLYYFESSDGNLVSKIQQSYSTGNYVTDGDKVTYADEQNNQVTGLALIDDQLQYFDPSDGRQVKNEQVIVDGVTYYFDKNGNGQYLFTNTATMSTNEFAKHSAAYSNDSSSFKNTIDGFLTADTWYRPKDILENGQTWVVSSTNDVRPLITVWWLNKDVQVNYSNFMKQNGLLDTSSQFNLQSDQYDLNVAAQKVQVAIEKRISKEKSTDWLKDLLFEAHEDTPSFVKQQFIWNKDSEYQGQGDAWFQGGYLKYGNNELTPTTNSDYRESGNTLDFLLANDVDNSNPAVQAENLNWLHYLMNFGTITANDDDANFDSIRIDAVDFIDNDAIQRTYDYMRDAYKVDASEDNANKHISLVEAGLDAGTSTIKSDALVESNFREAATLSLANQSGENSSLTNMLQDIDGGQIIADHANNATENESTPNYSIIHAHDKGIQEKVGAAITDVTGADWTNFTDDQLKEGLAAYYQDQRSTNKKYNIYNLPSIYALMLTNKDTVPRVYYGDMYQDDGQYMEKQSIYYDAIVSLMNTRKSYVSGGQTMDVDEHGLLKSVRFGKDAMTASDLGTNETRTEGVGVLVGNDSSLKLNDSDTVTLEMGAAHKNQKYRAALLTTSDGIVTYDADNDAPTIWTDDRGTLTFSNKEIAGQDYTSVQGFANSQVSGYLAVWVPVGASDDQDVRTAALTDANLDDKVLHSNAALDSNLIYEGFSNFQPKATTNDELTNVVIAKNANLFEKWGITSFEMAPQYRSSGDHTFLDSTIDNGYAFTDRYDLGFETPTKYGTDKDLRTAIKALHQSNMQVMADVVDNQVYNLSGQEVVSASRAGVYGNDVSTGFGTQLYAVNSVGGGKYQAQYGGEYLNELKQQYPDLFEAKTYDYWVKNYSNDGSDPYYTLSQNTRKDMPSSEVIKQWSAKYMNGTNVLGNGMGYVLKDWNTGEYFKIGEKNADFITN

SEQ ID NO: 71 - protein, 1637 aa, Fructobacillus tropaeoli
DDSQQSSTQIQSTQVTTALPAGGQYSTTNGGQSWNYLVNGVAIKGMYQDGQGQLRYFNFIDGTQVKGEFLSINGTYYYFDQNSGEGHLVPTQSNGHYTEIGNTGAWGYQNSNGELVKGIQNIDGQLRYFDENTGNQVKGGSATIGNKSYYFEPSQGTLTTTIDQVSDAQNANIRGLATVNGQLNYFDPTTGEQAKHKQVATNGATYYFNDSGVGTYLFTNVQNTPANDVSQHNAVNSTDTKDYTNTVDGFLTADTWYRPKYILDNGENWRASNEGEYRPFIMNWWPNKNVEVNYLKLMQNNNLLSSTVQYDLFTDQAILNQAAYQAQIAIEKRIKSEGSTDWLNTLLFGGDDSHPSFVKQQFIWNSDSESPWQGDAWFQGGYLKYGNSVMTPTSNSNYRQAGNAFDFLLANDVDNQNPIVQAEDLNWLYYLMNFGSITTNGLDNDSNFDSIRLDAVDFIHNDAIQRTYDYLRQAFNLTKNEATANQHLSLVEAGVDAGTTTYNSDGLIESNIRPLATDSLTNAPGKNASLSNLIKDVDSGEVIADHANFSTDDGIPNYSIIHAHDKGIQENVGAAITAATGADWTNFTTEQLEQGLDLYYQDQRSTNKKYNIYNLPSIYALMLTNKGTVPRVYYGDMYQDNGQYMQQKSLYYDAISSLMTARKQYVAGGQTMSVDENGLLKSVRFGKNAMTAQDTGDAETRTEGVGVIIGNDPSVKVADGQTVTLDMGAAHKNQAYRPLILTTSDGIQTYDSDENAPVVYTDDNGILTFSNQDINGQANTKIVGTLNPQVSGYLAVWVPVGASADQDARTAPSTQSTNDGKVLHTGAALDSNLIFEGFSNFQPMPTTHDEMTNVVISQNASQFAKWGITSFEMAPQYRSSEDHSFLDSTIDNGYAFSDRYDLGFGTPTKYGTDEDLRNAIKALHQNGMQVMADVVMNQLYSLNGKEVVSASRAGVYGNDVDLPFGTQLYVVNTTGGGEYQKKYGGAFLNIIKEKYPTLFDSESYDYYLKNYSDNGHGPAYMTTATATREAIPSDQPLKEWSAKYMNGTNILGLGMGYVLKDWNNGAYFKLSGTDTTLPQSLVALTGWNQNDDGTWSYYSTDTDDRVTGKQVIDGRTLLFDNQGNQIKGGWGENPDGTWSYYNADTGDRVIGEQVIDGRTLFFDNQGVQVKGGWGENYDGTWSYYNADTGDRVTGKQVIDGRTLLFDNRGVQVKGGWGENSDGTWSYYNADTGDRVTGNQLIGGRNLLFDNQGNQIKGGWDENPDGTWSYYNADTGDRVTGVQVIDGKQLLFDSNGIQVKNSWQKNANGTWSYYDANDGHLVPANSSNDGTSSSTQDSGNKSNQNPSSSSNAVNKTTGWIKNSDGTWSYLSAKSGQKVTGSQTIDGKQLLFDDHGVQIKGGWGKNADGTWSYYDANSGELTSTSDMSNVNPQQTTTTTNEQSTTNQPTDITKNSDGVYVYKNDSNKKAQGYLNDGSSWKWFNDGQLYTGFQNYMGAYYYFINGIRQQNQWENIWGLKYYVGDDGRTVEGIHAIDGHAYDFGTDGTFNVKGSASGYLNDGKSWMWYEGGNPYTGFRYYMDTYYWFENGVRQDNAWHQAWGLTYYTGADGRAVQGVQNINGKLYYFGNDGTFFMRTNQEV

(19) United States
(12) Patent Application Publication
(10) Pub. No.: US 2025/0002956 A1
(43) Pub. Date: Jan. 2, 2025

(54) ALPHA-1,3-GLUCAN GRAFT COPOLYMERS

(71) Applicant: NUTRITION & BIOSCIENCES USA 4, INC., Rochester, NY (US)

(72) Inventors: RONG GUAN, WILMINGTON, DE (US); JAMES JOSHUA OHANE, WEST CHESTER, PA (US); NIKITA ILTCHENKO, WILMINGTON, DE (US); YEFIM BRUN, WILMINGTON, DE (US); BRIAN MCCAULEY, WAYNE, PA (US); LAURIE A. HOWE, BEAR, DE (US); YOUGEN LI, PENNINGTON, NJ (US)

(21) Appl. No.: 18/604,878

(22) Filed: Mar. 14, 2024

Related U.S. Application Data
(63) Continuation of application No. 17/406,386, filed on Aug. 19, 2021, now Pat. No. 11,932,890.
Application No. 17/406,386 is a continuation of application No. 16/218,518, filed on Dec. 13, 2018, now Pat. No. 11,098,334.
(60) Provisional application No. 62/598,685, filed on Dec. 14, 2017.
(60) Provisional application No. 62/733,729, filed on Sep. 20, 2018.

(51) Int. Cl. C12P 19/08; C08B 37/00; C08B 37/02; C08J 5/18; C08L 5/02; C11D 3/37; C12P 19/18

(52) U.S. Cl. CPC C12P 19/08; C08B 37/0009; C08B 37/0021; C08J 5/18; C08L 5/02; C11D 3/3707; C12P 19/18; C08J 2305/00; C08J 2305/02

(57) ABSTRACT

Compositions are disclosed herein comprising a graft copolymer that comprises: (i) a backbone comprising dextran that has been modified with about 1%-25% alpha-1,2 branches, and (ii) one or more alpha-1,3-glucan side chains comprising at least about 50% alpha-1,3 glycosidic linkages. Further disclosed are reactions for producing such graft copolymers, as well as their use in derivatives, films and various other applications.

This application is a continuation of application Ser. No. 17/406,386 (Aug. 19, 2021) (now U.S. Pat. No. 11,932,890), which is a continuation of application Ser. No. 16/218,518 (filed Dec. 13, 2018) (now U.S. Pat. No. 11,098,334), which claims the benefit of U.S. Provisional Application Nos. 62/598,685 (filed Dec. 14, 2017) and 62/733,729 (filed Sep. 20, 2018). All of these prior applications are incorporated herein by reference in their entirety.

### FIELD

The present disclosure is in the field of polysaccharides. For example, the disclosure pertains to compositions comprising dextran/alpha-1,3-glucan graft copolymers, derivatives thereof, and various applications using these materials.

### REFERENCE TO SEQUENCE LISTING SUBMITTED ELECTRONICALLY

The official copy of the sequence listing is submitted electronically via EFS-Web or Patent Center as a file named CL6591USCNT2_SequenceListing.xml created on Sep. 4, 2024 and having a size of about 136 kilobytes. The sequence listing contained in this file is part of the specification and herein incorporated by reference in its entirety.

### BACKGROUND

Driven by a desire to use polysaccharides in various applications, researchers have explored for polysaccharides that are biodegradable and that can be made economically from renewably sourced feedstocks. One such polysaccharide is alpha-1,3-glucan, an insoluble glucan polymer characterized by having alpha-1,3-glycosidic linkages. This polymer has been prepared, for example, using a glucosyltransferase enzyme isolated from Streptococcus salivarius (Simpson et al., Microbiology 141:1451-1460, 1995). Also for example, U.S. Pat. No. 7,000,000 disclosed the preparation of a spun fiber from enzymatically produced alpha-1,3-glucan. Various other glucan materials have also been studied for developing new or enhanced applications. For example, U.S. Patent Appl. Publ. No. 2015/0232819 discloses enzymatic synthesis of several insoluble glucans having mixed alpha-1,3 and-1,6 linkages.

Despite this work, new forms of alpha-1,3-glucan are desired to enhance the economic value and performance characteristics of this material in various applications. Compositions comprising alpha-1,3-glucan in the form of a graft copolymer are presently disclosed to address this need.

### SUMMARY

In one embodiment, the present disclosure concerns a composition comprising a graft copolymer, or an ether-or ester-derivative thereof, wherein the graft copolymer comprises: (i) a backbone comprising dextran that has been modified with about 1%- 25% alpha-1,2 branches, and (ii) one or more alpha-1,3-glucan side chains comprising at least about 50% alpha-1,3 glycosidic linkages.

In another embodiment, the present disclosure concerns a method of producing a graft copolymer, the method comprising: (a) contacting at least (i) water, (ii) sucrose, (iii) dextran that has been modified with about 1%- 25% alpha-1,2 branches, and (iv) a glucosyltransferase enzyme that synthesizes alpha-1,3-glucan comprising at least about 50% alpha-1,3 glycosidic linkages, whereby a graft copolymer as presently disclosed is produced; and (b) optionally, isolating the graft copolymer produced in step (a).

In another embodiment, the present disclosure concerns a method for producing a film, the method comprising: (a) dissolving an insoluble graft copolymer as presently disclosed, or ether-or ester-derivative thereof, in a solvent to provide a solution; (b) contacting the solution with a surface; and (c) removing the solvent to form a film.

In another embodiment, the present disclosure concerns a method of producing alpha-1,3-glucan, the method comprising: (a) contacting at least (i) water, (ii) sucrose, (iii) dextran that has been modified with about 1%- 50% alpha-1,2 branches, and (iv) a glucosyltransferase enzyme that synthesizes alpha-1,3-glucan comprising at least about 50% alpha-1,3 glycosidic linkages, thereby providing an enzymatic reaction, whereby alpha-1,3-glucan comprising at least about 50% alpha-1,3 glycosidic linkages is produced, and (b) optionally, isolating the alpha-1,3-glucan produced in step (a).

### BRIEF DESCRIPTION OF THE SEQUENCES

TABLE 1
Summary of Nucleic Acid and Protein SEQ ID Numbers^{b}

| | Nucleic acid | Protein |
|---|---|---|
| Description | SEQ ID NO. | SEQ ID NO. |
| GTF 0874, Streptococcus sobrinus. The first 156 amino acids | 1 ^{a} | 2 |
| of the protein are deleted compared to GENBANK | | (1435 aa) |
| Identification No. 450874; a start methionine is included. | | |
| GTF 6855, Streptococcus salivarius SK126. The first 178 | 3 ^{a} | 4 |
| amino acids of the protein are deleted compared to | | (1341 aa) |
| GENBANK Identification No. 228476855 (Acc. No. | | |
| ZP_04061500.1); a start methionine is included. | | |
| GTF 2379, Streptococcus salivarius. The first 203 amino | 5 ^{a} | 6 |
| acids of the protein are deleted compared to GENBANK | | (1247 aa) |
| Identification No. 662379; a start methionine is included. | | |
| GTF 7527 or GTFJ, Streptococcus salivarius. The first 42 | 7 ^{a} | 8 |
| amino acids of the protein are deleted compared to | | (1477 aa) |
| GENBANK Identification No. 47527; a start methionine is | | |
| included. | | |
| GTF 1724, Streptococcus downei. The first 162 amino acids | 9 ^{a} | 10 |
| of the protein are deleted compared to GENBANK | | (1436 aa) |
| Identification No. 121724; a start methionine is included. | | |
| GTF 0544, Streptococcus mutans. The first 164 amino acids | 11 ^{a} | 12 |
| of the protein are deleted compared to GENBANK | | (1313 aa) |
| Identification No. 290580544; a start methionine is included. | | |
| GTF 5926, Streptococcus dentirousetti. The first 144 amino | 13 ^{a} | 14 |
| acids of the protein are deleted compared to GENBANK | | (1323 aa) |
| Identification No. 167735926; a start methionine is included. | | |
| GTF 4297, Streptococcus oralis. The first 228 amino acids of | 15 ^{a} | 16 |
| the protein are deleted compared to GENBANK Identification | | (1348 aa) |
| No. 7684297; a start methionine is included. | | |
| GTF 5618, Streptococcus sanguinis. The first 223 amino | 17 ^{a} | 18 |
| acids of the protein are deleted compared to GENBANK | | (1348 aa) |
| Identification No. 328945618; a start methionine is included. | | |
| GTF 2765, unknown Streptococcus sp. C150. The first 193 | 19 ^{a} | 20 |
| amino acids of the protein are deleted compared to | | (1340 aa) |
| GENBANK Identification No. 322372765; a start methionine is | | |
| included. | | |
| GTF 0427, Streptococcus sobrinus. The first 156 amino acids | 25 ^{a} | 26 |
| of the protein are deleted compared to GENBANK | | (1435 aa) |
| Identification No. 940427; a start methionine is included. | | |
| GTF 2919, Streptococcus salivarius PS4. The first 92 amino | 27 ^{a} | 28 |
| acids of the protein are deleted compared to GENBANK | | (1340 aa) |
| Identification No. 383282919; a start methionine is included. | | |
| GTF 2678, Streptococcus salivarius K12. The first 188 amino | 29 ^{a} | 30 |
| acids of the protein are deleted compared to GENBANK | | (1341 aa) |
| Identification No. 400182678; a start methionine is included. | | |
| GTF 3929, Streptococcus salivarius JIM8777. The first 178 | 33 ^{a} | 34 |
| amino acids of the protein are deleted compared to | | (1341 aa) |
| GENBANK Identification No. 387783929; a start methionine is | | |
| included. | | |
| GTF 3298, Streptococcus sp. C150. The first 209 amino | | 59 |
| acids of the protein are deleted compared to GENBANK | | (1242 aa) |
| Identification No. 322373298; a start methionine is included. | | |
| Wild type GTFJ, Streptococcus salivarius. GENBANK | | 60 |
| Identification No. 47527. | | (1518 aa) |
| Wild type GTF corresponding to GTF 2678, Streptococcus | | 61 |
| salivarius K12. | | (1528 aa) |
| Wild type GTF corresponding to GTF 6855, Streptococcus | | 62 |
| salivarius SK126. | | (1518 aa) |
| Wild type GTF corresponding to GTF 2919, Streptococcus | | 63 |
| salivarius PS4. | | (1431 aa) |
| Wild type GTF corresponding to GTF 2765, unknown | | 64 |
| Streptococcus sp. C150. | | (1532 aa) |
| Shorter version of GTF 7527, Streptococcus salivarius, (also | | 65 |
| referred to as “7527-NT” herein. The first 178 amino acids of | | (1341 aa) |
| the protein are deleted compared to GENBANK Identification | | |
| No. 47527; a start methionine is included. | | |
| GTF 8117, mature dextransucrase, Lactobacillus animalis | | 66 |
| KCTC 3501. | | (1614 aa) |
| GTF 6831_a, mature dextransucrase, Streptococcus | | 67 |
| salivarius M18. | | (1557 aa) |
| GTF 6831_b, mature dextransucrase, Streptococcus | | 68 |
| salivarius M18. | | (1552 aa) |
| GTF 5604, mature dextransucrase, Streptococcus criceti | | 69 |
| HS-6. | | (1302 aa) |
| GTFJ18T1, alpha-1,2 branching enzyme, Leuconostoc | | 70 |
| mesenteroides. The first 1664 amino acids of the protein are | | (1107 aa) |
| deleted compared to GENBANK Identification No. | | |
| WP_014324604.1. | | |
| GTF 9905, mature alpha-1,2 branching enzyme, | | 71 |
| Fructobacillus tropaeoli. | | (1637 aa) |

^{a} This DNA coding sequence is codon-optimized for expression in E. coli, and is merely disclosed as an example of a suitable coding sequence.
^{b}SEQ ID NOs: 21-24, 31, 32 and 35-58 are intentionally not included in this table and merely serve as placeholders.

### DETAILED DESCRIPTION

The disclosures of all cited patent and non-patent literature are incorporated herein by reference in their entirety.

Unless otherwise disclosed, the terms “a” and “an” as used herein are intended to encompass one or more (i.e., at least one) of a referenced feature.

Where present, all ranges are inclusive and combinable, except as otherwise noted. For example, when a range of “1 to 5” (i.e., 1-5) is recited, the recited range should be construed as including ranges “1 to 4”, “1 to 3”, “1-2”, “1-2 & 4-5”, “1-3 & 5”, and the like.

The term “copolymer” herein refers to a polymer comprising at least two different types of alpha-glucan, such as dextran and alpha-1,3-glucan.

The terms “graft copolymer”, “branched copolymer” and the like herein generally refer to a copolymer comprising a “backbone” (or “main chain”) and side chains branching from the backbone. The side chains are structurally distinct from the backbone. Examples of graft copolymers herein comprise a dextran backbone that has been modified with about 1%- 35% alpha-1,2 branches, and at least one side chain of alpha-1,3-glucan comprising at least about 50% alpha-1,3 glycosidic linkages. In some aspects, a dextran backbone can have an alpha-1,3-glucan extension, since the non-reducing end of dextran can prime alpha-1,3-glucan synthesis by a glucosyltransferase enzyme. A backbone can be an alpha-1,3-glucan-dextran linear copolymer in some instances.

The terms “alpha-1,3-glucan side chain” and “alpha-1,3-glucan arm” and the like can be used interchangeably herein. Alpha-1,3-glucan side chains are contemplated to be (i) extensions of alpha-1,2 branches, since alpha-1,2 branches present non-reducing ends that can possibly prime alpha-1,3-glucan synthesis by a glucosyltransferase enzyme; (ii) joined via alpha-1,3 linkage directly to the dextran backbone, in which case alpha-1,3-glucan side chain synthesis occurred by a glucosyltransferase enzyme first making an alpha-1,3 branch from the dextran backbone followed by extension of this branch by the enzyme; and/or (iii) joined via other alpha-glycosidic linkages (e.g., alpha-1,4 or alpha-1,2) to the dextran backbone (in some cases, such other linkages might result from the promiscuous activity of an alpha-1,3-glucan-synthesizing glucosyltransferase enzyme).

The terms “alpha-glucan”, “alpha-glucan polymer” and the like are used interchangeably herein. An alpha-glucan is a polymer comprising glucose monomeric units linked together by alpha-glycosidic linkages. In typical embodiments, an alpha-glucan herein comprises at least about 80%, 81%, 82%, 83%, 84%, 85%, 86%, 87%, 88%, 89%, 90%, 91%, 92%, 93%, 94%, 95%, 96%, 97%, 98%, 99%, or 100% alpha-glycosidic linkages. Examples of alpha-glucan polymers herein include graft copolymers as presently disclosed.

The terms “poly alpha-1,3-glucan”, “alpha-1,3-glucan”, “alpha-1,3-glucan polymer” and the like are used interchangeably herein. Alpha-1,3-glucan is a polymer comprising glucose monomeric units linked together by glycosidic linkages, wherein at least about 30% of the glycosidic linkages are alpha-1,3. Alpha-1,3-glucan in certain embodiments comprises at least about 90% or 95% alpha-1,3 glycosidic linkages. Most or all of the other linkages in alpha-1,3-glucan herein typically are alpha-1,6, though some linkages may also be alpha-1,2 and/or alpha-1,4. Alpha-1,3-glucan as presently defined can characterize an alpha-1,3-glucan side chain herein. In some aspects, alpha-1,3-glucan can characterize an alpha-1,3-glucan “homopolymer”, which is alpha-1,3-glucan that is not part of (i) a graft copolymer or (ii) part of a dextran-alpha-1,3-glucan linear copolymer.

The terms “dextran”, “dextran polymer”, “dextran molecule” and the like herein refer to a water-soluble alpha-glucan comprising at least 90% alpha-1,6 glycosidic linkages (with the balance of the linkages typically being alpha-1,3). Enzymes capable of synthesizing dextran from sucrose may be described as “dextransucrases” (EC 2.4.1.5).

A “substantially linear” dextran herein has 5% or less branches, before being modified to contain 1%- 25% alpha-1,2 branches. A “completely linear” dextran has no branches, before being modified to contain 1%- 25% alpha-1,2 branches. Dextran branches, if present prior to modification with alpha-1,2 branches, typically are short, being one (pendant) to three glucose monomers in length, and comprise less than about 10% of all the glucose monomers of a dextran polymer. Dextran as used in a glucosyltransferase reaction herein for alpha-1,3-glucan synthesis can optionally be characterized as a “primer”.

An “alpha-1,2 branch” (and like terms) as referred to herein comprises a glucose that is alpha-1,2-linked to a dextran backbone; thus, an alpha-1,2 branch herein can also be referred to as an alpha-1,2,6 linkage. An alpha-1.2 branch (before possible extension with an alpha-1,3-glucan-synthesizing glucosyltransferase) herein typically has one glucose group (can optionally be referred to as a pendant glucose).

An “alpha-1,3 branch” (and like terms) as referred to herein comprises a glucose that is alpha-1,3-linked to a dextran backbone; thus, an alpha-1,3 branch herein can also be referred to as an alpha-1.3.6 linkage. An alpha-1,3 branch herein is contemplated to possibly result from a glucosyltransferase enzyme first making an alpha-1,3 branch from the dextran backbone followed by extension of this branch by the enzyme.

The percent branching in an alpha-glucan herein refers to that percentage of all the linkages in the alpha-glucan that represent branch points. For example, the percent of alpha-1,2 branching in an alpha-glucan herein refers to that percentage of all the linkages in the glucan that represent alpha-1,2 branch points.

The terms “linkage”, “glycosidic linkage”, “glycosidic bond” and the like refer to the covalent bonds connecting the sugar monomers within a saccharide compound (oligosaccharides and/or polysaccharides). Examples of glycosidic linkages include alpha-linked glucose oligomers with 1,6-alpha-D-glycosidic linkages (herein also referred to as “alpha-1,6” linkages); 1,3-alpha-D-glycosidic linkages (herein also referred to as “alpha-1,3” linkages); 1,4-alpha-D-glycosidic linkages (herein also referred to as “alpha-1,4” linkages); and 1,2-alpha-D-glycosidic linkages (herein also referred to as “alpha-1,2” linkages); and combinations of such linkages typically associated with branched saccharide oligomers. The glycosidic linkages of a glucan polymer herein can also be referred to as “glucosidic linkages”. Herein, “alpha-D-glucose” is referred to as “glucose”. Alpha-1,2 linkages typically only occur at branch points, and do not occur in tandem (i.e., two or more consecutive glucose monomers are not joined by consecutive alpha-1,2 linkages).

The glycosidic linkage profile of an alpha-glucan herein can be determined using any method known in the art. For example, a linkage profile can be determined using methods using nuclear magnetic resonance (NMR) spectroscopy (e.g., ¹³C NMR or ¹H NMR). These and other methods that can be used are disclosed in, for example, Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, FL, 2005), which is incorporated herein by reference.

The “molecular weight” of large alpha-glucan polymers herein can be represented as weight-average molecular weight (Mw) or number-average molecular weight (Mn), the units of which are in Daltons or grams/mole. Alternatively, the molecular weight of large alpha-glucan polymers can be represented as DPw (weight average degree of polymerization) or DPn (number average degree of polymerization). The molecular weight of smaller alpha-glucan polymers such as oligosaccharides typically can be provided as “DP” (degree of polymerization), which simply refers to the number of glucoses comprised within the alpha-glucan; “DP” can also characterize the molecular weight of a polymer on an individual molecule basis. Various means are known in the art for calculating these various molecular weight measurements such as with high-pressure liquid chromatography (HPLC), size exclusion chromatography (SEC), or gel permeation chromatography (GPC).

The term “sucrose” herein refers to a non-reducing disaccharide composed of an alpha-D-glucose molecule and a beta-D-fructose molecule linked by an alpha-1,2-glycosidic bond. Sucrose is known commonly as table sugar. Sucrose can alternatively be referred to as “alpha-D-glucopyranosyl-(1→2)-beta-D-fructofuranoside”. “Alpha-D-glucopyranosyl” and “glucosyl” are used interchangeably herein. The terms “glucosyltransferase”, “glucosyltransferase enzyme”, “GTF”, “glucansucrase” and the like are used interchangeably herein. The activity of a glucosyltransferase herein catalyzes the reaction of the substrate sucrose to make the products alpha-glucan and fructose. Other products (by-products) of a GTF reaction can include glucose, various soluble gluco-oligosaccharides, and leucrose. Wild type forms of glucosyltransferase enzymes generally contain (in the N-terminal to C-terminal direction) a signal peptide (which is typically removed by cleavage processes), a variable domain, a catalytic domain, and a glucan-binding domain. A glucosyltransferase herein is classified under the glycoside hydrolase family 70 (GH70) according to the CAZy (Carbohydrate-Active EnZymes) database (Cantarel et al., Nucleic Acids Res. 37: D233-238, 2009). The term “dextransucrase” (and like terms) can optionally be used to characterize a glucosyltransferase enzyme that produces dextran. The term “alpha-1,2-branching enzyme” (and like terms) can optionally be used to characterize a glucosyltransferase enzyme that introduces one or more alpha-1,2 branches to a dextran backbone.

The term “glucosyltransferase catalytic domain” herein refers to the domain of a glucosyltransferase enzyme that provides alpha-glucan-synthesizing activity to a glucosyltransferase enzyme. A glucosyltransferase catalytic domain typically does not require the presence of any other domains to have this activity.

The terms “enzymatic reaction”, “glucosyltransferase reaction”, “glucan synthesis reaction”, “reaction composition”, “reaction formulation” and the like are used interchangeably herein and generally refer to a reaction that initially comprises water, sucrose, at least one active glucosyltransferase enzyme, and optionally other components such as dextran herein. Components that can be further present in a glucosyltransferase reaction typically after it has commenced include fructose, glucose, leucrose, soluble gluco-oligosaccharides (e.g., DP2-DP7) (such may be considered as products or by-products, depending on the glucosyltransferase used), and/or insoluble alpha-glucan product(s) of DP8 or higher. It would be understood that certain glucans products, such as alpha-1,3-glucan with a degree of polymerization (DP) of at least 8 or 9, are water-insoluble and thus not dissolved in a glucan synthesis reaction, but rather may be present out of solution (e.g., by virtue of having precipitated from the reaction). It is in a glucan synthesis reaction where the step of contacting water, sucrose and a glucosyltransferase enzyme is performed. The term “under suitable reaction conditions” as used herein refers to reaction conditions that support conversion of sucrose to alpha-glucan product(s) via glucosyltransferase enzyme activity. It is during such a reaction that glucosyl groups originally derived from the input sucrose are enzymatically transferred and used in alpha-glucan polymer synthesis; glucosyl groups as involved in this process can thus optionally be referred to as the glucosyl component or moiety (or like terms) of a glucosyltransferase reaction.

The “yield” of insoluble alpha-glucan product in a glucosyltransferase reaction in some aspects herein represents the molar yield based on the converted sucrose. The molar yield of an alpha-glucan product can be calculated based on the moles of insoluble alpha-glucan product divided by the moles of the sucrose converted. Moles of converted sucrose can be calculated as follows: (mass of initial sucrose−mass of final sucrose)/molecular weight of sucrose [342 g/mol]. This molar yield calculation can be considered as a measure of selectivity of the reaction toward the alpha-glucan. In some aspects, the “yield” of insoluble alpha-glucan product in a glucosyltransferase reaction can be based on the glucosyl component of the reaction. Such a yield (yield based on glucosyl) can be measured using the following formula:

$$Insoluble Alpha - Glucan Yield = ( ( IS / 2 - ( FS / 2 + LE / 2 + GL + SO ) ) / ( IS / 2 - FS / 2 ) ) \times 100 \% .$$

The fructose balance of a glucosyltransferase reaction can be measured to ensure that HPLC data, if applicable, are not out of range (90-110% is considered acceptable). Fructose balance can be measured using the following formula:

$$Fructose Balance = ( ( 180 / 342 \times ( FS + LE ) + FR ) / ( 180 / 342 \times IS ) ) \times 100 \% .$$

In the above two formulae, IS is [Initial Sucrose], FS is [Final Sucrose], LE is [Leucrose], GL is [Glucose], SO is [Soluble Oligomers] (gluco-oligosaccharides), and FR is [Fructose]; the concentrations of each foregoing substrate/product provided in double brackets are in units of grams/L and as measured by HPLC, for example.

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The terms “aqueous liquid”, “aqueous fluid” and the like as used herein can refer to water or an aqueous solution. An “aqueous solution” herein can comprise one or more dissolved salts, where the maximal total salt concentration can be about 3.5 wt % in some embodiments. Although aqueous liquids herein typically comprise water as the only solvent in the liquid, an aqueous liquid can optionally comprise one or more other solvents (e.g., polar organic solvent) that are miscible in water. Thus, an aqueous solution can comprise a solvent having at least about 10 wt % water.

An “aqueous composition” herein has a liquid component that comprises at least about 10 wt % water, for example. Examples of aqueous compositions include mixtures, solutions, dispersions (e.g., colloidal dispersions), suspensions and emulsions, for example. An aqueous composition in certain embodiments can comprise an insoluble form of graft copolymer as disclosed herein, in which case the aqueous composition can optionally be characterized as a solid-in-liquid composition, given the insolubility of the graft copolymer. As used herein, the term “colloidal dispersion” refers to a heterogeneous system having a dispersed phase and a dispersion medium, i.e., microscopically dispersed insoluble particles are suspended throughout another substance (e.g., an aqueous composition such as water or aqueous solution). An example of a colloidal dispersion herein is a hydrocolloid. All, or a portion of, the particles of a colloidal dispersion such as a hydrocolloid can comprise a graft copolymer herein. The terms “dispersant” and “dispersion agent” are used interchangeably herein to refer to a material that promotes the formation and/or stabilization of a dispersion.

A glucan (or ether-or ester-derivative thereof) that is “insoluble”, “aqueous-insoluble”, “water-insoluble” (and like terms) (e.g., alpha-1,3-glucan with a DP of 8 or higher) does not dissolve (or does not appreciably dissolve) in water or other aqueous conditions, optionally where the aqueous conditions are further characterized to have a pH of 4-9 (e.g., pH 6-8) and/or temperature of about 1 to 85° C. (e.g., 20-25° C.). In contrast, glucans (or ether-or ester-derivative thereof) such as certain oligosaccharides herein that are “soluble”, “aqueous-soluble”, “water-soluble” and the like (e.g., alpha-1,3-glucan with a DP less than 8) appreciably dissolve under these conditions.

The terms “film”, “sheet” and like terms herein refer to a thin, visually continuous material. A film can be comprised as a thin layer or coating on a material, or can be alone (e.g., not attached to a material surface; free-standing). A “coating” as used herein refers to a thin layer covering a surface of a material.

The term “uniform thickness” as used to characterize a film or coating herein can refer to a contiguous area that (i) is at least 20% of the total film/coating area, and (ii) has a standard deviation of thickness of less than about 50 nm, for example.

The terms “elongation at break”, “elongation”, “rupture elongation” and the like herein refer to the percent change in length of a film under stress, measured from the start of application of stress to the film to when the film breaks.

The term “viscosity” as used herein refers to the measure of the extent to which a fluid (aqueous or non-aqueous) resists a force tending to cause it to flow. Various units of viscosity that can be used herein include centipoise (cP, cps) and Pascal-second (Pa·s), for example. A centipoise is one one-hundredth of a poise; one poise is equal to 0.100 kg·m¹·s⁻¹.

The term “household care product” and like terms typically refer to products, goods and services relating to the treatment, cleaning, caring and/or conditioning of a home and its contents. The foregoing include, for example, chemicals, compositions, products, or combinations thereof having application in such care.

The terms “fabric”, “textile”, “cloth” and the like are used interchangeably herein to refer to a woven material having a network of natural and/or artificial fibers. Such fibers can be in the form of thread or yarn, for example.

A “fabric care composition” and like terms refer to any composition suitable for treating fabric in some manner. Examples of such a composition include laundry detergents and fabric softeners, which are examples of laundry care compositions.

The terms “heavy duty detergent”, “all-purpose detergent” and the like are used interchangeably herein to refer to a detergent useful for regular washing of white and/or colored textiles at any temperature. The terms “low duty detergent”, “fine fabric detergent” and the like are used interchangeably herein to refer to a detergent useful for the care of delicate fabrics such as viscose, wool, silk, microfiber or other fabric requiring special care. “Special care” can include conditions of using excess water, low agitation, and/or no bleach, for example.

A “detergent composition” herein typically comprises at least a surfactant (detergent compound) and/or a builder. A “surfactant” herein refers to a substance that tends to reduce the surface tension of a liquid in which the substance is dissolved. A surfactant may act as a detergent, wetting agent, emulsifier, foaming agent, and/or dispersant, for example.

The term “personal care product” and like terms typically refer to products, goods and services relating to the treatment, cleaning, cleansing, caring or conditioning of a person. The foregoing include, for example, chemicals, compositions, products, or combinations thereof having application in such care.

An “oral care composition” herein is any composition suitable for treating a soft or hard surface in the oral cavity such as dental (teeth) and/or gum surfaces.

Terms used herein regarding “ethers” (e.g., graft copolymer ether-derivative) are defined as in U.S. Patent Appl. Publ. Nos. 2014/179913, 2016/0304629, 2016/0311935, 2015/0239995, 2018/0230241 and/or 2018/0237816, which are incorporated herein by reference.

Terms used herein regarding “esters” (e.g., graft copolymer ester-derivative) are defined as in U.S. Patent Appl. Publ. Nos. 2014/0187767 and/or 2018/0155455, and/or Int. Patent Appl. Publ. No. WO2018/098065, which are incorporated herein by reference.

The term “degree of substitution” (DOS) as used herein refers to the average number of hydroxyl groups that are substituted (with organic groups via ether linkage, or with acyl groups via ester linkage) in each monomeric unit (glucose) of a graft copolymer ether-or ester-derivative herein.

The terms “sequence identity”, “identity” and the like as used herein with respect to a polypeptide amino acid sequence are as defined and determined in U.S. Patent Appl. Publ. No. 2017/0002336, which is incorporated herein by reference.

Various polypeptide amino acid sequences and polynucleotide sequences are disclosed herein as features of certain embodiments. Variants of these sequences that are at least about 70-85%, 85-90%, or 90%- 95% identical to the sequences disclosed herein can be used or referenced. Alternatively, a variant amino acid sequence or polynucleotide sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with a sequence disclosed herein. The variant amino acid sequence or polynucleotide sequence has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, or 99% of the function/activity of the disclosed sequence. Any polypeptide amino acid sequence disclosed herein not beginning with a methionine can typically further comprise at least a start-methionine at the N-terminus of the amino acid sequence. In contrast, any polypeptide amino acid sequence disclosed herein beginning with a methionine can optionally lack such a methionine residue.

The terms “aligns with”, “corresponds with”, and the like can be used interchangeably herein. Some embodiments herein relate to a glucosyltransferase comprising at least one amino acid substitution at a position corresponding with at least one particular amino acid residue of SEQ ID NO:62. An amino acid position of a glucosyltransferase or subsequence thereof (e.g., catalytic domain or catalytic domain plus glucan-binding domains) (can refer to such an amino acid position or sequence as a “query” position or sequence) can be characterized to correspond with a particular amino acid residue of SEQ ID NO:62 (can refer to such an amino acid position or sequence as a “subject” position or sequence) if (1) the query sequence can be aligned with the subject sequence (e.g., where an alignment indicates that the query sequence and the subject sequence [or a subsequence of the subject sequence] are at least about 30%, 40%, 50%, 60%, 70%, 80%, or 90% identical), and (2) if the query amino acid position directly aligns with (directly lines up against) the subject amino acid position in the alignment of (1). In general, one can align a query amino acid sequence with a subject sequence (SEQ ID NO: 62 or a subsequence of SEQ ID NO:62) using any alignment algorithm, tool and/or software described disclosed herein (e.g., BLASTP, ClustalW, ClustalV, Clustal-Omega, EMBOSS) to determine percent identity. Just for further example, one can align a query sequence with a subject sequence herein using the Needleman-Wunsch algorithm (Needleman and Wunsch, J. Mol. Biol. 48:443-453, 1970) as implemented in the Needle program of the European Molecular Biology Open Software Suite (EMBOSS [e.g., version 5.0.0 or later], Rice et al., Trends Genet. 16:276-277, 2000). The parameters of such an EMBOSS alignment can comprise, for example: gap open penalty of 10, gap extension penalty of 0.5, EBLOSUM62 (EMBOSS version of BLOSUM62) substitution matrix.

The numbering of particular amino acid residues of SEQ ID NO:62 herein is with respect to the full-length amino acid sequence of SEQ ID NO:62. The first amino acid (i.e., position 1, Met-1) of SEQ ID NO:62 is at the start of the signal peptide. Unless otherwise disclosed, substitutions herein are with respect to the full-length amino acid sequence of SEQ ID NO:62.

A “non-native glucosyltransferase” herein (“mutant”, “variant”, “modified” and like terms can likewise be used to describe such a glucosyltransferase) has at least one amino acid substitution at a position corresponding with a particular amino acid residue of SEQ ID NO: 62. Such at least one amino acid substitution typically is in place of the amino acid residue(s) that normally (natively) occurs at the same position in the native counterpart (parent) of the non-native glucosyltransferase (i.e., although SEQ ID NO:62 is used as a reference for position, an amino acid substitution herein is with respect to the native counterpart of a non-native glucosyltransferase) (considered another way, when aligning the sequence of a non-native glucosyltransferase with SEQ ID NO:62, determining whether a substitution exists at a particular position does not depend in-and-of-itself on the respective amino acid residue in SEQ ID NO:62, but rather depends on what amino acid exists at the subject position within the native counterpart of the non-native glucosyltransferase). The amino acid normally occurring at the relevant site in the native counterpart glucosyltransferase often (but not always) is the same as (or conserved with) the particular amino acid residue of SEQ ID NO:62 for which the alignment is made. A non-native glucosyltransferase optionally can have other amino acid changes (mutations, deletions, and/or insertions) relative to its native counterpart sequence.

It may be instructive to illustrate a substitution/alignment herein. SEQ ID NO:12 (GTF 0544) is a truncated form of a Streptococcus sobrinus glucosyltransferase. It is noted that Leu-193 of SEQ ID NO: 12 corresponds with Leu-373 of SEQ ID NO:62 (alignment not shown). If SEQ ID NO: 12 is mutated at position 193 to substitute the Leu residue with a different residue (e.g., Gln), then it can be stated that the position 193-mutated version of SEQ ID NO:12 represents a non-native glucosyltransferase having an amino acid substitution at a position corresponding with Leu-373 of SEQ ID NO:62, for example.

The term “isolated” means a substance (or process) in a form or environment that does not occur in nature. A non-limiting example of an isolated substance includes any non-naturally occurring substance such as a graft copolymer herein (as well as the enzymatic reactions and other processes used in preparation thereof). It is believed that the embodiments disclosed herein are synthetic/man-made (could not have been made except for human intervention/involvement), and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein.

New forms of alpha-1,3-glucan are desired to enhance the economic value and performance characteristics of this material in various applications. Compositions comprising alpha-1,3-glucan in the form of a graft copolymer are presently disclosed to address this need.

Certain embodiments of the present disclosure concern a composition comprising a graft copolymer, or an ether-or ester-derivative thereof, wherein said graft copolymer comprises:
(i) a backbone comprising dextran that has been modified with about 1%- 25% alpha-1,2 branches, and
(ii) one or more alpha-1,3-glucan side chains comprising at least about 50% alpha-1,3 glycosidic linkages.

A dextran that forms the backbone of a graft copolymer herein can comprise about 100% alpha-1,6-glycosidic linkages (i.e., completely linear dextran backbone), or about, or at least about, 95%, 96%, 97%, 98%, 99%, or 99.5% alpha-1,6-glycosidic linkages (i.e., substantially linear dextran backbone), for example. Such a percent alpha-1,6 linkage profile is that taking account of the total of all linkages in the dextran (combination of main chain and, if present, branch portions). In some aspects, a substantially linear dextran backbone can comprise 5%, 4%, 3%, 2%, 1%, 0.5% or less branches (as present before modification with alpha-1,2 branches). If present, dextran backbone branches typically are short, being one (pendant) to three glucose monomers in length, and comprise less than about 10%, 9%, 8%, 7%, 6%, 5%, 4%, 3%, 2%, or 1% of all the glucose monomers of the dextran backbone. All the foregoing dextran backbone linkage information pertains to a dextran backbone prior to its modification with alpha-1,2 branches.

A backbone of a graft copolymer in certain embodiments can be comprised entirely of a dextran as presently disclosed. However, in some aspects, a backbone can comprise other elements. For example, a graft copolymer backbone can comprise alpha-1,3-glucan originating from the non-reducing side of a dextran main chain, by virtue of the main chain (at its non-reducing end) serving to prime alpha-1,3-glucan synthesis during synthesis of the graft copolymer.

In some aspects, a dextran backbone, prior to alpha-1,2 branch modification, can have a DP or DPw of about, or at least about, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 85, 90, 95, 100, 105, 110, 150, 200, 250, 300, 400, or 500. This DP or DPw can optionally be expressed as a range between any two of these values. Merely as examples, the DP or DPw can be about 8-20, 8-30, 8-100, or 8-500, 3-4, 3-5, 3-6, 3-7, 3-8, 4-5, 4-6, 4-7, 4-8, 5-6, 5-7, 5-8, 6-7, 6-8, or 7-8. Merely as other examples, this DP or DPw can be 90-120, 95-120, 100-120, 105-120, 110-120, 115-120, 90-115, 95-115, 100-115, 105-115, 110-115, 90-110, 95-110, 100-110, 105-110, 90-105, 95-105, 100-105, 90-100, 95-100, 90-95, 85-95, or 85-90. Yet, in some aspects, a dextran backbone, prior to alpha-1,2 branch modification, can be any as disclosed in U.S. Patent Appl. Publ. Nos. 2016/0122445, 2017/0218093, or 2018/0282385, or International Patent Appl. Publ. Nos. WO2017/079595, WO2015/183714, or WO2017/091533, for example, which are all incorporated herein by reference.

Dextran herein can be produced enzymatically, for example, prior to being modified with alpha-1,2-branches. In certain embodiments, dextran can be synthesized using a dextransucrase and/or methodology as disclosed in International Patent Appl. Publ. Nos. WO2015/183714 or WO2017/091533, or U.S. Patent Appl. Publ. No. 2018/0282385, which are all incorporated herein by reference. The dextransucrase identified as GTF8117 (SEQ ID NO: 66 herein), GTF6831 (SEQ ID NO:67 or 68 herein), or GTF5604 (SEQ ID NO:69 herein) in these references can be used, if desired (or any dextransucrase comprising an amino acid sequence that is at least about 90%, 91%, 92%, 93%, 94%, 95%, 96%, 97%, 98%, or 99% identical to any of these particular dextransucrases). Such enzymatically produced dextran is linear (i.e., 100% alpha-1,6 linkages) and aqueous soluble.

A dextran that forms the backbone of a graft copolymer herein has been modified with about 1%- 25% alpha-1,2 branches. These alpha-1,2 branches are distinct from any branches that may have been present in dextran prior to modification with alpha-1,2 branches. In embodiments employing a linear dextran as a substrate for alpha-1,2 branching, the backbone of a graft copolymer can simply be characterized to comprise about 1%- 25% alpha-1,2 branches (or any alpha-1,2 branching percentage disclosed herein). Such a backbone thus comprises only alpha-1,6 and-1,2 linkages (alpha-1,2,6) (i.e., an alpha-1,6-linked backbone decorated with alpha-1,2-linked pendant glucoses), with no other linkage types present.

The percent alpha-1,2 branching of a backbone of a graft copolymer herein can be about, at least about, or less than about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%, for example. This alpha-1,2 branching profile can optionally be expressed as having a range between any two of these values. Merely as examples, the percentage of alpha-1,2 branches can be about 2-25%, 2-20%, 2-15%. 2-10%, 5-25%, 5-20%, 5-15%, 5-10%, 7-13%, 8-12%, 9-11%, 10-25%, 10-20%, or 10-15%.

One or more alpha-1,2 branches of a backbone herein are typically a single glucose monomer in length (i.e., they are pendant glucoses). All of, or at least 80% or 90% of, the alpha-1,2 branches are a single glucose monomer in length, for instance.

In certain aspects, the DP or DPw of a backbone of a graft copolymer modified with about 1%- 25% alpha-1,2 branches is about 10 to 500. This DP or DPw can be about, or at least about, 10, 20, 30, 40, 50, 60, 70, 80, 85, 90, 95, 100, 105, 110, 115, 120, 125, 130, 140, 150, 160, 170, 180, 190, 200, 300, 400, or 500, for example, and can optionally be expressed as a range between any two of these values. Merely as examples, the DPw of a backbone herein can be about 85-130, 85-125, 85-120, 85-115, 85-110, 85-105, 85-100, 85-95, 85-90, 90-130, 90-125, 90-120, 90-115, 90-110, 90-105, 90-100, 90-95, 95-130, 95-125, 95-120, 95-115, 95-110, 95-105, 95-100, 100-130, 100-125, 100-120, 100-115, 100-110, 100-105, 105-130, 105-125, 105-120, 105-115, 105-110, 110-130, 110-125, 110-120, 110-115, 115-130, 115-125, 115-120, 120-130, or 120-125.

The modification/addition of alpha-1,2 branches to dextran herein is typically done enzymatically. In certain embodiments, alpha-1,2 branches can be synthesized off of dextran using an alpha-1,2 branching enzyme and/or methodology as disclosed in International Patent Appl. Publ. Nos. WO2015/183714 or WO2017/091533, or U.S. Patent Appl. Publ. No. 2018/0282385, which are all incorporated herein by reference. The branching enzyme identified as GTFJ18T1 (SEQ ID NO:70 herein) or GTF9905 (SEQ ID NO: 71 herein) in these references can be used, if desired (or any alpha-1,2 branching enzyme comprising an amino acid sequence that is at least about 90%, 91%, 92%, 93%, 94%, 95%, 96%, 97%, 98%, or 99% identical to any of these particular branching enzymes). Such an enzymatically produced alpha-1,2-branched dextran is aqueous soluble.

A graft copolymer as presently disclosed comprises one or more alpha-1,3-glucan side chains comprising at least about 50% alpha-1,3 glycosidic linkages. These side chains typically result via reacting an alpha-1,2-branched dextran herein with a glucosyltransferase that can synthesize alpha-1,3-glucan. For clarity purposes, these side chains ought not be considered dextran branches per se.

An alpha-1,3-glucan side chain in certain aspects can comprise about, or at least about, 50%, 60%, 70%, 80%, 85%, 90%, 95%, 96%, 97%, 98%, 99%, 99.5%, or 100% alpha-1,3 glycosidic linkages. In some aspects, accordingly, an alpha-1,3-glucan side chain has less than about 50%, 40%, 30%, 20%, 15%, 10%, 9%, 8%, 7%, 6%, 5%, 4%, 3%, 2%, 1%, 0.5%, or 0% glycosidic linkages that are not alpha-1,3. Typically, the glycosidic linkages that are not alpha-1,3 are mostly or entirely alpha-1,6. It should be understood that the higher the percentage of alpha-1,3 linkages present in a side chain, the greater the probability that the side chain is linear, since there are lower occurrences of certain linkages that might be part of branch points in the side chain. In certain embodiments, an alpha-1,3-glucan side chain has no branch points or less than about 5%, 4%, 3%, 2%, or 1% branch points as a percent of the glycosidic linkages in the side chain. Glucosyltransferases contemplated to be useful for producing alpha-1,3-glucan side chains comprising at least about 50% alpha-1,3 linkages as above are disclosed in U.S. Pat. Nos. 7,000,000 and 8,871,474, and Int. Patent Appl. Publ. No. WO2017/079595, all of which are incorporated herein by reference. In aspects in which a backbone comprises 50% alpha-1,3 glycosidic linkages, such a backbone typically does not comprise alternan (alternating alpha-1,3 and-1,6 linkages).

The DP of one or more alpha-1,3-glucan side chains in certain aspects can individually be about, or at least about, 100, 200, 300, 400, 500, 600, 700, 800, 900, 1000, 1100, or 1200. DP can optionally be expressed as a range between any two of these values. Merely as examples, the DP of one or more alpha-1,3-glucan side chains can individually be about 400-1200, 500-1200, 600-1200, 700-1200, 400-1100, 500-1100, 600-1100, 700-1100, 400-1000, 500-1000, 600-1000, 700-1000, 400-900, 500-900, 600-900, or 700-900. As more examples, the DP of one or more alpha-1,3-glucan side chains can individually (i) be about, or less than about, 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 29, 28, 27, 26, 25, 24, 23, 22, 21, 20, 19, 18, 17, 16, 15, 14, 13, 12, or 11, or (ii) range from 11-25, 12-25, 11-22, 12-22, 11-20, 12-20, 20-300, 20-200, 20-150, 20-100, 20-75, 30-300, 30-200, 30-150, 30-100, 30-75, 50-300, 50-200, 50-150, 50-100, 50-75, 75-300, 75-200, 75-150, 75-100, 100-300, 100-200, 100-150, 150-300, 150-200, or 200-300. The DPw of a plurality of alpha-1,3-glucan side chains of a graft copolymer can be referred to, if desired; any of the foregoing DP values, which characterize side chains on an individual basis, can optionally be considered a DPw of all the side chains of a copolymer. In some aspects in which a graft copolymer has a plurality of alpha-1,3-glucan side chains, the individual DP values of the side chains are similar to each other (e.g., the DP values vary by less than about 2.5%, 5%, 10%, 15%, or 20%).

In some embodiments, an alpha-1,3-glucan side chain can comprise at least about 30% alpha-1,3 linkages and a percentage of alpha-1,6 linkages that brings the total of both the alpha-1,3 and-1,6 linkages in the side chain to 100%. For example, the percentage of alpha-1,3 and-1,6 linkages can be about 30-40% and 60-70%, respectively. Glucosyltransferases contemplated to be useful for producing alpha-1,3-glucan side chains comprising at least about 30% alpha-1,3 linkages are disclosed in U.S. Patent Appl. Publ. No. 2015/0232819, which is incorporated herein by reference.

One or more alpha-1,3-glucan side chains in some aspects are contemplated to be (A) extensions of alpha-1,2 branches, since alpha-1,2 branches present non-reducing ends that can possibly prime alpha-1,3-glucan synthesis by a glucosyltransferase enzyme, and/or (B) joined via alpha-1,3 linkage directly to the dextran backbone, in which case alpha-1,3-glucan side chain synthesis occurred by a glucosyltransferase enzyme first making an alpha-1,3 branch from the dextran backbone followed by extension of this branch by the enzyme. In some aspects, alpha-1,3-glucan side chains are all linked to the dextran backbone via the linkage type of (A) or (B), or via a combination of both (A) and (B) linkage types. Regarding the latter, a combination of both linkage types can comprise about 5%, 10%, 15%, 20%, 25%, 30%, 35%, 40%, 45%, 50%, 55%, 60%, 65%, 70%, 75%, 80%, 85%, 90%, or 95% of linkage type (A), with the balance of the other linkage types being of type (B).

The number of alpha-1,3-glucan side chains of a graft copolymer herein can be about, or at least about, 1, 2, 3, 4, 5, 6, 7, 8, 9, 10, 11, 12, 13, 14, 15, 16, 17, 18, 19, or 20, for example, or a range between any two of these values. Merely as examples, the number of alpha-1,3-glucan side chains can be 1-20, 1-15, 1-10, 1-5, 1-6, 2-5, 2-6, 3-5, 3-6, 4-5, 4-6, or 5-6. In some aspects, the number of alpha-1,3-glucan side chains can be up to about 10%, 20%, 30%, 40%, 50%, 60%, 70%, 80%, or 90% of the number of glucose monomers of the dextran portion of the graft copolymer.

A graft copolymer as presently disclosed typically is aqueous insoluble. Such insolubility can be in non-caustic aqueous conditions, such as the conditions of a glucosyltransferase reaction herein (e.g., pH 4-8, see below). In some aspects, a graft copolymer is insoluble in aqueous conditions at a temperature up to about 50, 60, 70, 80, 90, 100, 110, or 120° C. However, a graft copolymer can be aqueous soluble in some aspects. An aqueous composition herein such as an aqueous solution can comprise a solvent having about, or at least about, 10, 20, 30, 40, 50, 60, 70, 80, 90, or 100 wt % water, for example.

The DPw of a graft copolymer herein can be the sum of the DPw of any alpha-1,2-branched dextran backbone herein plus the DPw of any alpha-1,3-glucan side chain herein (taking into account the number of chains), for example. Merely as examples, the DPw of a graft copolymer herein can be about 1000-3000, 1500-3000, 2000-3000, 2500-3000, 1000-2500, 1500-2500, 2000-2500, 1000-2000, 1500-2000, 1000-1500, 200-1400, 200-1200, 200-1000, 200-800, 200-600, 200-400, 400-1400, 400-1200, 400-1000, 400-800, 400-600, 600-1400, 600-1200, 600-1000, or 600-800.

A graft copolymer herein can comprise about, or at least about, 1%, 2%, 3%, 4%, 5%, 6%, 7%, 8%, 9%, 10%, 11% or 12% (e.g., by weight) (or a range between any two of these values) of alpha-1,2-branched dextran backbone, for example. Merely as examples, a graft copolymer can comprise about 1-12%, 1-10%, 1-8%, 1-6%, 2-12%, 2-10%, 2-8%, 2-6%, 4-12%, 4-10%, 4-8%, or 4-6% (e.g., by weight) of alpha-1,2-branched dextran backbone, for example, or a percent (±0.5%) as reported in the below Examples (“% primer”).

A graft copolymer as presently disclosed can be a product of any of the enzymatic reaction processes disclosed below, for example.

Certain embodiments of the present disclosure concern a method of producing (preparing) a graft copolymer as described herein. Such a graft polymer production method can comprise: (a) contacting at least (i) water, (ii) sucrose, (iii) dextran that has been modified with about 1%- 25% alpha-1,2 branches, and (iv) a glucosyltransferase enzyme that synthesizes alpha-1,3-glucan comprising at least about 50% alpha-1,3 glycosidic linkages, thereby producing a graft copolymer as presently disclosed; and (b) optionally, isolating the graft copolymer produced in step (a). Step (a) can optionally be characterized as performing a reaction (or preparing/providing a reaction composition) comprising at least water, sucrose, dextran that has been modified with about 1%- 25% alpha-1,2 branches, and a glucosyltransferase enzyme that synthesizes alpha-1,3-glucan with at least about 50% alpha-1,3 glycosidic linkages. A graft polymer production method herein can optionally further comprise, prior to step (a), modifying dextran with alpha-1,2 branches (i.e., adding alpha-1,2 branches to dextran) to provide dextran with about 1%- 25% alpha-1,2 branches (for use in step [a]). Any of the foregoing features of a graft polymer production method herein (e.g., alpha-1,2-branched dextran, alpha-1,3-glucan, graft copolymer) can be as described elsewhere herein, such as above or in the below Examples. For example, the dextran used can be substantially linear dextran or completely linear dextran (prior to modification with about 1-25% alpha-1,2 branches). A method herein of producing a graft copolymer can also be characterized as a method of producing alpha-1,3-glucan, if desired.

A glucosyltransferase enzyme for producing alpha-1,3-glucan side chains of a graft copolymer herein can be derived from any microbial source, such as bacteria. Examples of bacterial glucosyltransferase enzymes are those derived from a Streptococcus species, Leuconostoc species or Lactobacillus species. Examples of Streptococcus species include S. salivarius, S. sobrinus, S. dentirousetti, S. downei, S. mutans, S. oralis, S. gallolyticus and S. sanguinis. Examples of Leuconostoc species include L. mesenteroides, L. amelibiosum, L. argentinum, L. carnosum, L. citreum, L. cremoris, L. dextranicum and L. fructosum. Examples of Lactobacillus species include L. acidophilus, L. delbrueckii, L. helveticus, L. salivarius, L. casei, L. curvatus, L. plantarum, L. sakei, L. brevis, L. buchneri, L. fermentum and L. reuteri.

A glucosyltransferase enzyme for producing alpha-1,3-glucan side chains of a graft copolymer herein can in some aspects comprise an amino acid sequence as disclosed in any of U.S. Patent Appl. Publ. Nos. 2014/0087431, 2017/0166938, 2017/0002335 and 2018/0072998 (corresponds to Patent. application Ser. No. 15/702,893), and U.S. patent application Ser. No. 16/127,288, all of which are incorporated herein by reference. In some aspects, a glucosyltransferase enzyme herein can comprise an amino acid sequence that is 100% identical to, or at least 90%, 91%, 92%, 93%, 94%, 95%, 96%, 97%, 98%, 98.5%, 99%, or 99.5% identical to, SEQ ID NO:2, 4, 6, 8, 10, 12, 14, 16, 18, 20, 26, 28, 30, 34, or 59 (Table 1), and have glucosyltransferase activity. It is noted that a glucosyltransferase enzyme with SEQ ID NO:2, 4, 8, 10, 14, 20, 26, 28, 30, or 34 can synthesize alpha-1,3-glucan side chains comprising at least about 90% (˜100%) alpha-1,3 linkages.

The amino acid sequence of a glucosyltransferase enzyme in certain aspects has been modified such that the enzyme produces more products (alpha-1,3-glucan and fructose), and less by-products (e.g., glucose, oligosaccharides such as leucrose), from a given amount of sucrose substrate. For example, one, two, three, four, or more amino acid residues of the catalytic domain of a glucosyltransferase herein can be modified/substituted to obtain an enzyme that produces more products (insoluble alpha-glucan and fructose). Examples of a suitable modified glucosyltransferase enzyme are disclosed in the below Examples (Tables A and B). A modified glucosyltransferase enzyme, for example, can comprise one or more amino acid substitutions corresponding with those in Tables A and/or B (or in Table 3 of U.S. Patent Appl. Publ. No. 2018/0072998 [incorporated herein by reference, corresponding to patent application Ser. No. 15/702,893] ) that is/are associated with an alpha-1,3-glucan yield of at least 40% (the position numbering of such at least one substitution corresponds with the position numbering of SEQ ID NO:62). A set of amino acid modifications as listed in Tables A or B can be used, for example.

The amino acid sequence of a glucosyltransferase enzyme in certain aspects has been modified such that the enzyme produces alpha-1,3-glucan with a molecular weight (DPw) that is lower than the molecular weight of alpha-1,3-glucan produced by its corresponding parent glucosyltransferase. Examples of a suitable modified glucosyltransferase enzyme are disclosed in the below Examples (Tables C and D). A modified glucosyltransferase enzyme, for example, can comprise one or more amino acid substitutions corresponding with those in Tables C and/or D that is/are associated with an alpha-1,3-glucan product molecular weight that is at least 5% less than the molecular weight of alpha-1,3-glucan produced by parent enzyme (the position numbering of such at least one substitution corresponds with the position numbering of SEQ ID NO:62). A set of amino acid modifications as listed in Table D can be used, for example.

The amino acid sequence of a glucosyltransferase enzyme in certain aspects has been modified such that the enzyme produces alpha-1,3-glucan with a molecular weight (DPw) that is higher than the molecular weight of alpha-1,3-glucan produced by its corresponding parent glucosyltransferase. Examples of a suitable modified glucosyltransferase enzyme are disclosed in the below Examples (Tables E and F). A modified glucosyltransferase enzyme, for example, can comprise one or more amino acid substitutions corresponding with those in Tables E and/or F that is/are associated with an alpha-1,3-glucan product molecular weight that is at least 5% higher than the molecular weight of alpha-1,3-glucan produced by parent enzyme (the position numbering of such at least one substitution corresponds with the position numbering of SEQ ID NO:62). A set of amino acid modifications as listed in Table 5 of U.S. patent application Ser. No. 16/127,288 can be used, for example.

In some aspects, a modified glucosyltransferase (i) comprises at least one amino acid substitution or a set of amino acid substitutions (as described above regarding yield or molecular weight), and (ii) comprises or consists of a glucosyltransferase catalytic domain that is at least about 90%, 91%, 92%, 93%, 94%, 95%, 96%, 97%, 98%, 99%, or 99.5% identical to amino acid residues 55-960 of SEQ ID NO:4, residues 54-957 of SEQ ID NO:65, residues 55-960 of SEQ ID NO:30, residues 55-960 of SEQ ID NO:28, or residues 55-960 of SEQ ID NO:20. Each of these subsequences are the approximate catalytic domains of each respective reference sequence, and are believed to be able to produce alpha-1,3-glucan comprising at least about 50% (e.g., ≥90% or ≥95%) alpha-1,3 linkages, and optionally further have a DPw of at least 100. In some aspects, a modified glucosyltransferase (i) comprises at least one amino acid substitution or a set of amino acid substitutions (as described above), and (ii) comprises or consists of an amino acid sequence that is at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 69%, 70%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to SEQ ID NO:62 or a subsequence thereof such as SEQ ID NO:4 (without start methionine thereof) or positions 55-960 of SEQ ID NO:4 (approximate catalytic domain).

The temperature of a reaction composition herein can be controlled, if desired, and can be about 5-50° C., 20-40° C., 30-40° C., 20-30° C., 20-25° C., 20° C., 25° C., 30° C., 35° C., or 40° C., for example.

The initial concentration of sucrose in a reaction composition herein can be about, or at least about, 10, 20, 30, 40, 45, 50, 55, 60, 80, 90, 95, 100, 105, 110, 125, 150, 200, 300, 400, 500, or 600 g/L, or a range between any two of these values. Merely as examples, the initial sucrose concentration can be about 10-150, 40-60, 45-55, 90-110, or 95-105 g/L, for example. “Initial concentration of sucrose” refers to the sucrose concentration in a reaction composition just after all the reaction components have been added/combined (e.g., at least water, sucrose, dextran with about 1%- 35% alpha-1,2 branches, glucosyltransferase enzyme).

The initial concentration of an alpha-1,2-branched dextran as presently disclosed in a reaction composition can be about, or at least about, 0.1, 0.5, 1, 2, 2.5, 3, 4, 5, 6, 7, 8, 9, 10, 11, 12, 13, 14, 15, 16, 17, 18, 19, or 20 g/L, or a range between any two of these values. Merely as examples, the initial concentration of an alpha-1,2-branched dextran can be about 0.5-12, 1-12, 2-12, 4-12, 0.5-10, 1-10, 2-10, 4-10, 0.5-8, 1-8, 2-8, 4-8, 0.5-6, 1-6, 2-6, or 4-6 g/L.

In some aspects, the ratio, by weight, of alpha-1,2-branched dextran to sucrose is about 1:100 to about 1:5, or about 1:50 to about 1:5. Such a ratio is with respect to a reaction composition upon its initial setup.

The pH of a reaction composition in certain embodiments can be about 4.0-9.0, 4.0-8.5, 4.0-8.0, 5.0-8.0, 5.5-7.5, or 5.5-6.5. In some aspects, the pH can be about 4.0, 4.5, 5.0, 5.5, 6.0, 6.5, 7.0, 7.5, or 8.0. The pH can be adjusted or controlled by the addition or incorporation of a suitable buffer, including but not limited to: phosphate, tris, citrate, or a combination thereof. The buffer concentration in a reaction composition herein can be about 0.1-300 mM, 0.1-100 mM, 10-100 mM, 5 mM, 10 mM, 20 mM, or 50 mM, for example.

A reaction composition can be contained within any vessel (e.g., an inert vessel/container) suitable for applying one or more of the reaction conditions disclosed herein. An inert vessel in some aspects can be of stainless steel, plastic, or glass (or comprise two or more of these components) and be of a size suitable to contain a particular reaction. For example, the volume/capacity of an inert vessel (and/or the volume of a reaction composition herein), can be about, or at least about, 1, 10, 50, 100, 500, 1000, 2500, 5000, 10000, 12500, 15000, or 20000 liters. An inert vessel can optionally be equipped with a stirring device. Any of the foregoing features, for example, can be used to characterize an isolated reaction herein.

A reaction composition herein can contain one, two, or more different glucosyltransferase enzymes that produce alpha-1,3-glucan side chains, for example. In some embodiments, only one or two glucosyltransferase enzymes is/are comprised in a reaction composition. A reaction composition herein can be, and typically is, cell-free (e.g., no whole cells present).

Completion of a reaction in certain aspects can be determined visually (e.g., no more accumulation of insoluble graft copolymer product), and/or by measuring the amount of sucrose left in the solution (residual sucrose), where a percent sucrose consumption of at least about 90%, 95%, or 99% can indicate reaction completion. In some aspects, a reaction can be considered complete when its sucrose content is at or below about 2-5 g/L. A reaction of the disclosed process can be conducted for about 1 hour to about, or at least about, 2, 4, 6, 8, 10, 12, 14, 16, 18, 20, 22, 24, 36, 48, 60, 72, 96, 120, 144, or 168 hours, for example. A reaction can optionally be terminated and/or otherwise treated to stop glucosyltransferase activity by heating it to at least about 65° C. for at least about 30-60 minutes.

Examples of other conditions and/or components suitable for synthesizing alpha-1,3-glucan side chains in graft copolymer production herein are disclosed in U.S. Patent Appl. Publ. Nos. 2014/0087431, 2017/0166938 and 2017/0002335, which are incorporated herein by reference.

Graft copolymer produced in a reaction composition herein can optionally be isolated. In certain embodiments, isolating graft copolymer can include at least conducting a step of centrifugation, filtration, fractionation, chromatographic separation, dialysis, evaporation, or dilution. Isolation of insoluble graft copolymer can include at least conducting a step of preparing a cake of graft copolymer. Cake preparation can include at least conducting a step of centrifugation (cake is pelleted graft copolymer) and/or filtration (cake is filtered graft copolymer). Isolation can optionally further comprise washing the centrifuged and/or filtered graft copolymer one, two, or more times with water or other aqueous liquid. A wash volume can optionally be at least about 10-100% of the volume of the reaction composition used to produce the graft copolymer, for example. Washing can be done by various modes, as desired, such as by displacement or re-slurry washing. In some aspects, the aqueous portion of the resulting cake has no (detectable) dissolved sugars, or about 0.1-1.5, 0.1-1.25, 0.1-1.0, 0.1-0.75, 0.1-0.5, 0.2-0.6, 0.3-0.5, 0.2, 0.3, 0.4, 0.5, or 0.6 wt % dissolved sugars. Such dissolved sugars can include sucrose, fructose, leucrose, and/or soluble gluco-oligosaccharides, for example. Isolation herein can optionally further comprise drying graft copolymer, and/or preparing a dispersion of graft copolymer. Preparing a dispersion of graft copolymer in some aspects can comprise: preparing a wet cake of insoluble graft copolymer (above), and dispersing the wet cake in water or an aqueous solution. Suitable means for dispersing wet cake herein include, for example, using a suitable dispersal tool such as a disperser, sonicator (e.g., ultrasonicator), homomixer, homogenizer (e.g., rotary or piston, rotar-stator), microfluidizer, planetary mixer, colloid mill, jet mill, vortex, and/or any methodology as described in International Patent Appl. Publ. Nos. WO2016/126685 or WO2016/030234, U.S. Pat. Nos. 5,767,176, 6,139,875, or 8,722,092, or U.S. Patent Appl. Publ. Nos. 2017/0055540, 2018/0021238, or 2017/0281780, all of which publications are incorporated herein by reference.

An isolated graft copolymer herein (or derivative thereof) as provided in a dry form, can comprise no more than 2.0, 1.5, 1.0, 0.5, 0.25, 0.10, 0.05, or 0.01 wt % water, for example. In some aspects, a graft copolymer product (or derivative thereof) is provided in an amount of at least 1 gram (e.g., at least about 2.5, 5, 10, 25, 50, 100, 250, 500, 750, 1000, 2500, 5000, 7500, 10000, 25000, 50000, or 100000 g); such an amount can be a dry amount, for example.

In some aspects, a graft copolymer (or derivative thereof) that has been isolated (optionally characterized as “purified”) can be present in a composition at a wt % (dry weight basis) of at least about 50%, 60%, 70%, 80%, 85%, 90%, 91%, 92%, 93%, 94%, 95%, 96%, 97%, 98%, 98.5%, 99%, 99.5%, 99.8%, or 99.9%. Such isolated graft copolymer (or derivative thereof) can be used as an ingredient/component in a product/application, for example.

In some aspects, the yield of alpha-1,3-glucan as comprised in a graft copolymer product (i.e., alpha-1,3-glucan primed with alpha-1,2-branched dextran herein) of a reaction composition herein can be about, or at least about, 3%, 4%, 5%, 6%, 7%, 8%, 9%, 10%, 3-10%, 3-9%, 3-8%, 4-10%, 4-9%, or 4-8% higher than the yield of alpha-1,3-glucan synthesized in a reaction lacking a dextran primer. This is notable, since the yield of alpha-1,3-glucan in a reaction composition comprising a dextran primer lacking alpha-1,2 branches was observed in the below Examples to be at most about 2.5% higher than the yield of alpha-1,3-glucan synthesized in a reaction lacking a dextran primer (e.g., consider Tables 5 and 11). Typically, such a yield comparison can be made under otherwise identical or similar reaction conditions (e.g., initial sucrose conc., temperature, pH, and/or reaction time), and using any suitable measurement technique (e.g., HPLC or NIR spectroscopy). In some aspects, such a yield comparison can be made with respect to alpha-1,3-glucan primed with dextran having about 5-15%, 8-12%, or 9-10% alpha-1,2 branches, and/or with respect to reactions comprising an 80-120, 90-120, or 100-110 g/L initial sucrose concentration. Alpha-1,3-glucan yield in some aspects can be measured based on the glucosyl component of the reaction.

A composition of the present disclosure can be a film or coating, for example. A film or coating can be a dried film or coating in some aspects, comprising less than about 3, 2, 1, 0.5, or 0.1 wt % water, for example. The amount of graft copolymer (or derivative thereof, such as a graft copolymer ester) comprised in a film or coating herein can be about, or at least about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, or 99.9 wt %, for example.

A film or coating herein can have a thickness of about, or at least about, 0.5, 0.6, 0.7, 0.8, 0.9, 1.0, 1.1, 1.2, 1.3, 1.4, 1.5, 0.5-1.5, 0.8-1.5, 1.0-1.5, 0.5-1.4, 0.8-1.4, or 1.0-1.4 mil (0.001 inch), for instance. In some aspects, such thickness is uniform, which can be characterized by having a contiguous area that (i) is at least 20%, 30%, 40%, or 50% of the total film/coating area, and (ii) has a standard deviation of thickness of less than about 0.06, 0.05, or 0.04 mil. A film or coating herein can be characterized as thin in some aspects. A film herein is typically a cast film.

A film or coating herein can exhibit various degrees of transparency as desired. For example, a film/coating can be highly transparent (e.g., high optical transparency, and/or low haze). Optical transparency as used herein can refer to a film or coating allowing at least about 10-99% light transmission, or at least about 50%, 60%, 70%, 80%, 90%, 95%, 96%, 97%, 98%, or 99% light transparency, for example. High transparency can optionally refer to a film/coating having at least about 90% optical transmittance. Transparency of a film/coating herein can be measured following test ASTM D 1746 (2009, Standard Test Method for Transparency of Plastic Sheeting, ASTM International, West Conshohocken, PA), for example, which is incorporated herein by reference.

A film or coating herein can optionally further comprise a plasticizer such as glycerol, propylene glycol, ethylene glycol, and/or polyethylene glycol. In certain embodiments, other film components (in addition to a graft copolymer herein) can be as disclosed in U.S. Patent. Appl. Publ. No. 2011/0151224 or 2015/0191550, or U.S. Pat. No. 9,688,035 or 3,345,200, all of which are incorporated herein by reference.

A film in certain aspects can exhibit a percent elongation at break of about, or at least about, 20%, 22%, 24%, 26%, 28%, 30%, 32%, 34%, 36%, 20-36%, 24-36%, or 28-36%. A film in certain aspects can exhibit a toughness of about, or at least about, 250, 300, 350, 400, 450, 500, 550, 600, 250-600, 300-600, 350-600, or 400-600 in-lb./in3. A film in certain aspects can exhibit a WTM (“work-to-max”, maximum amount of work applied to film before its breakage) of about, or at least about, 0.15, 0.16, 0.17, 0.18, 0.19, 0.20, 0.22, 0.24, 0.26, 0.28, 0.30, or 0.32 in-lb./in. In some aspects, a film herein can have one or more features (value +5-10% of value) of the graft copolymer-containing films tested in Table 14 (below), and/or of a film as disclosed in U.S. Pat. Nos. 9,688,035 or 5,750,204, which are incorporated herein by reference. Any of the foregoing film features, or any other applicable film features, can be measured optionally following methodology disclosed in Example 7, and/or as disclosed in U.S. Pat. No. 9,688,035 or U.S. Patent. Appl. Publ. No. 2015/0191550, which are both incorporated herein by reference.

The present disclosure also regards a method of producing a film, which method comprises: (a) dissolving an aqueous-insoluble graft copolymer herein, or ether-or ester-derivative thereof, in a solvent to provide a solution; (b) contacting the solution with a surface; and (c) coagulating the solution to form a film. This method can be practiced, for example, following methodology described below in Example 7 and/or as disclosed in U.S. Patent. Appl. Publ. No. 2015/0191550, which is incorporated herein by reference. In some aspects, a film production method can comprise a step (c) of removing the solvent, which removal can be done for example by drying and/or coagulation.

For the preparation of a film herein, a solution of graft copolymer in a solvent is prepared. A solvent can comprise, for example, aqueous NaOH (e.g., 4-15 wt % or 10 wt % NaOH), aqueous KOH (e.g., 4-15 wt % KOH), aqueous tetraethyl ammonium hydroxide (e.g., 20 wt %), or a mixture of DMSO (dimethyl sulfoxide) and LiCl (3-5 wt % LiCl). A solution can comprise about, or at least about, 3, 4, 5, 6, 7, 8, 9, 10, 11, 12, 13, 14, 15, 16, 17, 18, 19, 20, 21, 22, 23, 8-13, 9-13, 10-13, 8-12, 9-12, or 10-12 wt % graft copolymer, for example. In some aspects, a solution comprises less than 15, 14 or 13 wt %, graft copolymer. In some aspects of dissolving graft copolymer in a solvent, a slurry of graft copolymer in water can first be made, followed by addition of concentrated aqueous base.

A solution (for preparing a film) comprising a graft copolymer herein can further comprise additives such as a solubility additive or a rheology modifier, if desired. Examples of additives include urea and glycerol; the amount of either of these additives can be up to the wt % the graft copolymer in the solution, for example. Other additives can optionally be mixed into a graft copolymer solution, such as other polymers (e.g., in cases where a polymer blend is desired).

A graft copolymer solution is contacted with a surface/substrate during a film production process as presently disclosed. For film casting or extrusion, the viscosity of a graft copolymer solution typically is low enough to be flowable, but high enough to form a continuous film without breaking up. In some aspects, a solution concentration of less than 15 wt % (e.g., 3-15 wt %) graft copolymer is suitable for casting a film herein, whereas higher graft copolymer concentrations (e.g., 15-23 wt %) can be more suitable for extruding a film. A film can be produced by casting the graft copolymer solution onto a surface/substrate using a rod coater or a draw-down coater, for example. A suitable surface/substrate herein can be glass (optionally coated with a surfactant) or a polyester film. In some aspects, a film can be produced by extrusion through a slot die.

Solvent is removed from a graft copolymer solution (thereby forming a film) following its being contacted with a surface/substrate. In some aspects, solvent can be removed by drying and/or coagulating the graft copolymer solution. A film can be formed by directly immersing a graft copolymer solution (e.g., as cast onto a surface) in a coagulation media (thereby removing the solvent), or by first subjecting the solution to a drying step to remove a portion of the solvent, followed by coagulation to remove any remaining solvent. Solvent removal can be performed at room temperature (˜20-25° C.) or any suitable temperature between 20-80° C., for example. Coagulation media can comprise a non-solvent for the graft copolymer, such as water, alcohol (e.g., methanol), acid (e.g., sulfuric acid or acetic acid), or a mixture of two or more thereof; one or more salts can also be included, if desired. In some aspects, a coagulation medium can comprise water and about 10-20 wt % (e.g., ˜14 wt %) sulfuric acid and about 20-30 wt % (e.g., ˜25 wt %) sodium sulfate.

A film can optionally be washed following coagulation; water or alcohol (e.g., methanol) can be used for washing, for example. If desired, washing can be done until a neutral pH (e.g., pH 6-8, or ˜7) (of the wash) is achieved. Washing, or a post-washing step, can optionally further include bathing the film in a 1-10 wt % (e.g., ˜5 wt %) plasticizer (e.g., glycerol or ethylene glycol) solution (e.g., water- or alcohol-based) for a suitable period of time (e.g., at least 2, 3, or 4 minutes). A film herein is typically dried, such as by exposing it at a temperature of about 70-85° C. (e.g., ˜80° C.) for a suitable period of time (e.g., 10-20, or ˜15 minutes).

A composition in some aspects of the present disclosure can comprise an ether derivative of a graft copolymer herein (i.e., a graft copolymer can be derivatized to be ether-linked to one or more different organic groups). The degree of substitution (DoS) of a graft copolymer with one or more etherified organic groups can be about 0.001 to about 3.0, for example. The DoS in some aspects can be about, or at least about, or up to about, 0.001, 0.0025, 0.005, 0.01, 0.025, 0.05, 0.075, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 (DoS can optionally be expressed as a range between any two of these values). An ether group can be anionic, uncharged, or cationic; the charge of an ether group can be as it exists when the graft copolymer ether derivative is in an aqueous composition herein, further taking into account the pH of the aqueous composition and whether any salts are present.

An organic group etherified to a graft copolymer herein can comprise an alkyl group such as a methyl, ethyl, propyl, butyl, pentyl, hexyl, heptyl, octyl, nonyl, or decyl group, for example. In some aspects, an organic group etherified to a graft copolymer can be a substituted alkyl group in which there is a substitution on one or more carbons of the alkyl group. The substitution(s) may be one or more hydroxyl, aldehyde, ketone, and/or carboxyl groups. For example, a substituted alkyl group can be a hydroxy alkyl group, dihydroxy alkyl group, or carboxy alkyl group. Examples of suitable hydroxy alkyl groups include hydroxymethyl, hydroxyethyl, hydroxypropyl, hydroxybutyl and hydroxypentyl groups. Other examples include dihydroxy alkyl groups (diols) such as dihydroxymethyl, dihydroxyethyl, dihydroxypropyl, dihydroxybutyl and dihydroxypentyl groups. Examples of suitable carboxy alkyl groups include carboxymethyl (—CH₂COOH), carboxyethyl, carboxypropyl, carboxybutyl and carboxypentyl groups. An organic group in some aspects can comprise an aryl group such as a benzyl group.

An organic group etherified to a graft copolymer herein can be a positively charged (cationic) organic group in some aspects. A positively charged group can be, for example, any of those as disclosed in U.S. Patent Appl. Publ. No. 2016/0311935, which is incorporated herein by reference. A positively charged group can comprise a substituted ammonium group, for example. Examples of substituted ammonium groups are primary, secondary, tertiary and quaternary ammonium groups. An ammonium group can be substituted with one, two, or three alkyl groups (e.g., methyl, ethyl, propyl, butyl, pentyl, hexyl, heptyl, octyl, nonyl, decyl), for example. One of the groups of a substituted ammonium group comprises one carbon, or a chain of carbons, in ether linkage to a graft copolymer; such a carbon or carbon chain can be —CH₂—, —CH₂CH—, —CH₂CH₂CH₂—, —CH₂CH₂CH₂CH₂—, or —CH₂CH₂CH₂CH₂CH₂—, for example. A carbon or carbon chain in this context can optionally have at least one substitution with an oxygen atom (e.g., alcohol group) and/or alkyl group (e.g., methyl, ethyl, propyl, butyl). One or more positively charged organic groups in some aspects can be trimethylammonium hydroxypropyl groups (structure I, when each of R₂, R₃ and R₄ is a methyl group).

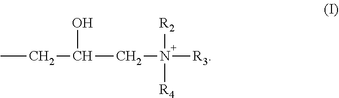

A graft copolymer ether in certain embodiments can contain one type of organic group. A non-limiting example of such a compound is carboxymethyl graft copolymer or benzyl graft copolymer. Alternatively, a graft copolymer ether compound can contain two or more different types of organic groups (e.g., both carboxymethyl and benzyl groups). In some aspects, a graft copolymer ether can comprise at least one nonionic organic group and at least one anionic group as ether groups. In some aspects, a graft copolymer ether can comprise at least one nonionic organic group and at least one positively charged organic group as ether groups. Thus, a graft copolymer ether herein can optionally be amphiphilic.

Any graft copolymer as presently disclosed is suitable for preparing an ether compound. Any suitable process for ether-derivatizing polysaccharides can be employed, such as disclosed in U.S. Pat. Nos. 2,961,439, 2,344,179, 2,203,703, 2,203,704, 2,380,879 and 2,974,134, U.S. Patent Appl. Publ. Nos. 2014/179913, 2016/0304629, 2016/0311935, 2015/0232785, 2015/0239995, 2018/0230241 and 2018/0237816, all of which are incorporated herein by reference.

A composition in some aspects of the present disclosure can comprise an ester derivative of a graft copolymer herein (i.e., a graft copolymer can be derivatized to be ester-linked to one or more different acyl groups). The DoS of a graft copolymer with one or more acyl groups can be about 0.001 to about 3.0, for example. The DoS in some aspects can be about, or at least about, or up to about, 0.001, 0.0025, 0.005, 0.01, 0.025, 0.05, 0.075, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 (DoS optionally be expressed as a range between any two of these values).

An acyl group can be, for example, any of those as disclosed in U.S. Patent Appl. Publ. Nos. 2014/0187767 and 2018/0155455, and Int. Patent Appl. Publ. No. WO2018/098065, which are incorporated herein by reference. Examples of acyl groups herein include methanoyl (formyl), ethanoyl (acetyl), propanoyl (propionyl), butanoyl (butyryl), pentanoyl (valeryl), hexanoyl (caproyl), heptanoyl (enanthyl), octanoyl (caprylyl), nonanoyl (pelargonyl), decanoyl (capryl), undecanoyl, dodecanoyl (lauroyl), tridecanoyl, tetradecanoyl (myristyl), pentadecanoyl, hexadecanoyl (palmityl), heptadecanoyl, octadecanoyl (stearyl), nonadecanoyl, eicosanoyl (arachidyl), uneicosanoyl, docosanoyl (behenyl), tricosanoyl, tetracosanoyl (lignoceryl), pentacosanoyl and hexacosanoyl (cerotyl) groups, for example. Additional examples of acyl groups herein include branched acyl groups (e.g., 2-methylpropanoyl, 2-methylbutanoyl, 2,2-dimethylpropanoyl, 3-methylbutanoyl, 2-methylpentanoyl, 3-methylpentanoyl group, 4-methylpentanoyl, 2,2-dimethylbutanoyl, 2,3-dimethylbutanoyl, 3,3-dimethylbutanoyl group, 2-ethylbutanoyl group, 2-ethylhexanoyl), cyclic acyl groups (e.g., cyclopropanoyl, cyclobutanoyl, cyclopentanoyl, cyclohexanoyl, cycloheptanoyl) and aryl acyl groups (e.g., benzoyl). Additional examples of acyl groups herein include

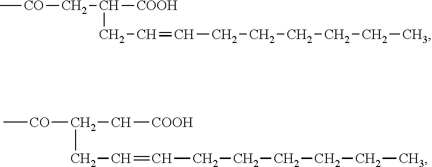 [SMILES: CCCCCCC=CCC(CC(C)=O)C(=O)O ; CCCCCCC=CCC(CC(=O)O)C(C)=O]

and any other acyl group that can be formed using a cyclic organic anhydride as an ester-derivatization agent.

A graft copolymer ester in certain embodiments can contain one type of acyl group (e.g., acetyl group or benzoyl group). Alternatively, a graft copolymer ester compound can contain two or more different types of acyl groups.

Any graft copolymer as presently disclosed is suitable for preparing an ester compound. Any suitable process for ester-derivatizing polysaccharides can be employed, such as disclosed in U.S. Patent Appl. Publ. Nos. 2014/0187767 and 2018/0155455, and Int. Patent Appl. Publ. No. WO2018/098065, which are incorporated herein by reference.

A graft copolymer herein, or ether-or ester-derivative thereof (non-derivatized and derivatized graft copolymers are collectively referred to herein as “graft copolymer compounds”) can be present in a composition, such as an aqueous composition (e.g., colloidal dispersion, solution) or dry composition, at about, at least about, or less than about, 0.01, 0.05, 0.1, 0.2, 0.3, 0.4, 0.5, 0.6, 0.7, 0.8, 0.9, 1.0, 1.2, 1.4, 1.6, 1.8, 2.0, 2.5, 3.0, 3.5, 4.0, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 wt %, for example. The liquid component of an aqueous composition can be water or an aqueous solution, for instance. The solvent of an aqueous solution typically is water, or can comprise about, or at least about, 10, 20, 30, 40, 50, 60, 70, 80, 90, 95, or 98 wt % water, for example.

An aqueous solution in some aspects has no (detectable) dissolved sugars, or about 0.1-1.5, 0.1-1.25, 0.1-1.0, 0.1-0.75, 0.1-0.5, 0.2-0.6, 0.3-0.5, 0.2, 0.3, 0.4, 0.5, or 0.6 wt % dissolved sugars. Such dissolved sugars can include sucrose, fructose, leucrose, and/or soluble gluco-oligosaccharides, for example. An aqueous solution in some aspects can have one or more salts/buffers (e.g., Na⁺, Cl⁻, NaCl, phosphate, tris, citrate) (e.g., ≤0.1, 0.5, 1.0, 2.0, or 3.0 wt %) and/or a pH of about 4.0, 4.5, 5.0, 5.5, 6.0, 6.5, 7.0, 7.5, 8.0, 8.5, 9.0, 4.0-9.0, 4.0-8.5, 4.0-8.0, 5.0-9.0, 5.0-8.5, 5.0-8.0, 6.0-9.0, 6.0-8.5, or 6.0-8.0, for example.

A composition comprising a graft copolymer compound of the present disclosure can, in some aspects, have a viscosity of about, or at least about, 10, 25, 50, 100, 250, 500, 750, 1000, 2000, 3000, 4000, 5000, 6000, 7000, 8000, 9000, 10000, 11000, 12000, 13000, 14000, 15000, 20000, 30000, 40000, 50000, 60000, 70000, 80000, 90000, 100000, 250000, 500000, or 1000000 centipoise (cP). Viscosity can be measured using a viscometer or rheometer, or using any other means known in the art. A rotational shear rate of about, or at least about, 10, 60, 150, 250, 600, 800, 1000, or 10-1000 rpm (revolutions per minute), for example, can be applied when measuring the viscosity of a composition herein. Viscosity can be measured at any suitable temperature (e.g., between 4-30, 20-30, 20-25, or 3-110° C.), and/or at any suitable pressure (e.g., atmospheric pressure, about 760 torr).

A composition comprising a graft copolymer compound of the present disclosure can, in some aspects, be non-aqueous (e.g., a dry composition). Examples of such embodiments include powders, granules, microcapsules, flakes, or any other form of particulate matter. Other examples include larger compositions such as pellets, bars, kernels, beads, tablets, sticks, or other agglomerates. A non-aqueous or dry composition typically has less than 3, 2, 1, 0.5, or 0.1 wt % water comprised therein.

A composition comprising a graft copolymer compound of the present disclosure can, in some aspects, comprise one or more salts such as a sodium salt (e.g., NaCl,

Na2SO4). Other non-limiting examples of salts include those having (i) an aluminum, ammonium, barium, calcium, chromium (II or III), copper (I or II), iron (II or III), hydrogen, lead (II), lithium, magnesium, manganese (II or III), mercury (I or II), potassium, silver, sodium strontium, tin (II or IV), or zinc cation, and (ii) an acetate, borate, bromate, bromide, carbonate, chlorate, chloride, chlorite, chromate, cyanamide, cyanide, dichromate, dihydrogen phosphate, ferricyanide, ferrocyanide, fluoride, hydrogen carbonate, hydrogen phosphate, hydrogen sulfate, hydrogen sulfide, hydrogen sulfite, hydride, hydroxide, hypochlorite, iodate, iodide, nitrate, nitride, nitrite, oxalate, oxide, perchlorate, permanganate, peroxide, phosphate, phosphide, phosphite, silicate, stannate, stannite, sulfate, sulfide, sulfite, tartrate, or thiocyanate anion. Thus, any salt having a cation from (i) above and an anion from (ii) above can be in a composition, for example. A salt can be present in an aqueous composition herein at a wt % of about, or at least about, 0.01, 0.025,. 05, 0.075, 0.1, 0.25, 0.5, 0.75, 1.0, 1.25, 1.5, 1.75, 2.0, 2.5, 3.0, 3.5, 0.01-3.5, 0.5-3.5, 0.5-2.5, or 0.5-1.5 wt % (such wt % values typically refer to the total concentration of one or more salts), for example.

A composition herein comprising a graft copolymer compound may optionally contain one or more active enzymes. Non-limiting examples of suitable enzymes include proteases, cellulases, hemicellulases, peroxidases, lipolytic enzymes (e.g., metallolipolytic enzymes), xylanases, lipases, phospholipases, esterases (e.g., arylesterase, polyesterase), perhydrolases, cutinases, pectinases, pectate lyases, mannanases, keratinases, reductases, oxidases (e.g., choline oxidase), phenoloxidases, lipoxygenases, ligninases, pullulanases, tannases, pentosanases, malanases, beta-glucanases, arabinosidases, hyaluronidases, chondroitinases, laccases, metalloproteinases, amadoriases, glucoamylases, arabinofuranosidases, phytases, isomerases, transferases and amylases. If an enzyme(s) is included, it may be comprised in a composition herein at about 0.0001-0.1 wt % (e.g., 0.01-0.03 wt %) active enzyme (e.g., calculated as pure enzyme protein), for example. In fabric care applications, cellulase can be present in an aqueous composition in which a fabric is treated (e.g., wash liquor) at a concentration that is minimally about 0.01-0.1 ppm total cellulase protein, or about 0.1-10 ppb total cellulase protein (e.g., less than 1 ppm), to maximally about 100, 200, 500, 1000, 2000, 3000, 4000, or 5000 ppm total cellulase protein, for example.

A composition comprising a graft copolymer compound herein, such as an aqueous composition or a non-aqueous composition (above), can be in the form of a household care product, personal care product, industrial product, pharmaceutical product, or food product, for example, such as described in any of U.S. Patent Appl. Publ. Nos. 2018/0022834, 2018/0237816, 2018/0230241, 20180079832, 2016/0311935, 2016/0304629, 2015/0232785, 2015/0368594, 2015/0368595, or 2016/0122445, or International Patent Appl. Publ. Nos. WO2016/160737, WO2016/160738, WO2016/133734, or WO2016/160740, which are all incorporated herein by reference. In some aspects, a composition comprising a graft copolymer compound can comprise at least one component/ingredient of a household care product, personal care product, industrial product, pharmaceutical product, or food product as disclosed in any of the foregoing publications and/or as presently disclosed.

Graft copolymer compounds disclosed herein are believed to be useful for providing one or more of the following physical properties to a personal care product, pharmaceutical product, household product, industrial product, or food product: thickening, freeze/thaw stability, lubricity, moisture retention and release, texture, consistency, shape retention, emulsification, binding, suspension, dispersion, gelation, reduced mineral hardness, for example. Examples of a concentration or amount of a graft copolymer compound in a product can be any of the weight percentages provided herein, for example.

Personal care products herein are not particularly limited and include, for example, skin care compositions, cosmetic compositions, antifungal compositions, and antibacterial compositions. Personal care products herein may be in the form of, for example, lotions, creams, pastes, balms, ointments, pomades, gels, liquids, combinations of these and the like. The personal care products disclosed herein can include at least one active ingredient, if desired. An active ingredient is generally recognized as an ingredient that causes an intended pharmacological effect.

In certain embodiments, a skin care product can be applied to skin for addressing skin damage related to a lack of moisture. A skin care product may also be used to address the visual appearance of skin (e.g., reduce the appearance of flaky, cracked, and/or red skin) and/or the tactile feel of the skin (e.g., reduce roughness and/or dryness of the skin while improved the softness and subtleness of the skin). A skin care product typically may include at least one active ingredient for the treatment or prevention of skin ailments, providing a cosmetic effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these. A skin care product may include one or more natural moisturizing factors such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example. Other ingredients that may be included in a skin care product include, without limitation, glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil.

A personal care product herein can also be in the form of makeup, lipstick, mascara, rouge, foundation, blush, eyeliner, lip liner, lip gloss, other cosmetics, sunscreen, sun block, nail polish, nail conditioner, bath gel, shower gel, body wash, face wash, lip balm, skin conditioner, cold cream, moisturizer, body spray, soap, body scrub, exfoliant, astringent, scruffing lotion, depilatory, permanent waving solution, antidandruff formulation, antiperspirant composition, deodorant, shaving product, pre-shaving product, after-shaving product, cleanser, skin gel, rinse, dentifrice composition, toothpaste, or mouthwash, for example. An example of a personal care product (e.g., a cleanser, soap, scrub, cosmetic) comprises a carrier or exfoliation agent (e.g., jojoba beads [jojoba ester beads]) (e.g., about 1-10, 3-7, 4-6, or 5 wt %); such an agent may optionally be dispersed within the product.

A personal care product in some aspects can be a hair care product. Examples of hair care products herein include shampoo, hair conditioner (leave-in or rinse-out), cream rinse, hair dye, hair coloring product, hair shine product, hair serum, hair anti-frizz product, hair split-end repair product, mousse, hair spray, and styling gel. A hair care product can be in the form of a liquid, paste, gel, solid, or powder in some embodiments. A hair care product as presently disclosed typically comprises one or more of the following ingredients, which are generally used to formulate hair care products: anionic surfactants such as polyoxyethylenelauryl ether sodium sulfate; cationic surfactants such as stearyltrimethylammonium chloride and/or distearyltrimethylammonium chloride; nonionic surfactants such as glyceryl monostearate, sorbitan monopalmitate and/or polyoxyethylenecetyl ether; wetting agents such as propylene glycol, 1,3-butylene glycol, glycerin, sorbitol, pyroglutamic acid salts, amino acids and/or trimethylglycine; hydrocarbons such as liquid paraffins, petrolatum, solid paraffins, squalane and/or olefin oligomers; higher alcohols such as stearyl alcohol and/or cetyl alcohol; superfatting agents; antidandruff agents; disinfectants; anti-inflammatory agents; crude drugs; water-soluble polymers such as methyl cellulose, hydroxycellulose and/or partially deacetylated chitin; antiseptics such as paraben; ultra-violet light absorbers; pearling agents; pH adjustors; perfumes; and pigments.

A pharmaceutical product herein can be in the form of an emulsion, liquid, elixir, gel, suspension, solution, cream, or ointment, for example. Also, a pharmaceutical product herein can be in the form of any of the personal care products disclosed herein, such as an antibacterial or antifungal composition. A pharmaceutical product can further comprise one or more pharmaceutically acceptable carriers, diluents, and/or pharmaceutically acceptable salts. A graft copolymer compound disclosed herein can also be used in capsules, encapsulants, tablet coatings, and as an excipients for medicaments and drugs.

A household and/or industrial product herein can be in the form of drywall tape-joint compounds; mortars; grouts; cement plasters; spray plasters; cement stucco; adhesives; pastes; wall/ceiling texturizers; binders and processing aids for tape casting, extrusion forming, injection molding and ceramics; spray adherents and suspending/dispersing aids for pesticides, herbicides, and fertilizers; fabric care products such as fabric softeners and laundry detergents; hard surface cleaners; air fresheners; polymer emulsions; gels such as water-based gels; surfactant solutions; paints such as water-based paints; protective coatings; adhesives; sealants and caulks; inks such as water-based ink; metal-working fluids; films or coatings; or emulsion-based metal cleaning fluids used in electroplating, phosphatizing, galvanizing and/or general metal cleaning operations, for example.

A graft copolymer compound disclosed herein can be comprised in a personal care product, pharmaceutical product, household product, or industrial product in an amount that provides a desired degree of thickening and/or dispersion, for example. Examples of a concentration or amount of a graft copolymer compound in a product can be any of the weight percentages provided above, for example.

Compositions disclosed herein can be in the form of a fabric care composition. A fabric care composition herein can be used for hand wash, machine wash and/or other purposes such as soaking and/or pretreatment of fabrics, for example. A fabric care composition may take the form of, for example, a laundry detergent; fabric conditioner; any wash-, rinse-, or dryer-added product; unit dose or spray. Fabric care compositions in a liquid form may be in the form of an aqueous composition as disclosed herein. In other aspects, a fabric care composition can be in a dry form such as a granular detergent or dryer-added fabric softener sheet. Other non-limiting examples of fabric care compositions herein include: granular or powder-form all-purpose or heavy-duty washing agents; liquid, gel or paste-form all-purpose or heavy-duty washing agents; liquid or dry fine-fabric (e.g. delicates) detergents; cleaning auxiliaries such as bleach additives, “stain-stick”, or pre-treatments; substrate-laden products such as dry and wetted wipes, pads, or sponges; sprays and mists.

A detergent composition herein may be in any useful form, e.g., as powders, granules, pastes, bars, unit dose, or liquid. A liquid detergent may be aqueous, typically containing up to about 70 wt % of water and 0 wt % to about 30 wt % of organic solvent. It may also be in the form of a compact gel type containing only about 30 wt % water.

A detergent composition herein typically comprises one or more surfactants, wherein the surfactant is selected from nonionic surfactants, anionic surfactants, cationic surfactants, ampholytic surfactants, zwitterionic surfactants, semi-polar nonionic surfactants and mixtures thereof. In some embodiments, the surfactant is present at a level of from about 0.1% to about 60%, while in alternative embodiments the level is from about 1% to about 50%, while in still further embodiments the level is from about 5% to about 40%, by weight of the detergent composition. A detergent will usually contain 0 wt % to about 50 wt % of an anionic surfactant such as linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl-or alkenylsuccinic acid, or soap. In addition, a detergent composition may optionally contain 0 wt % to about 40 wt % of a nonionic surfactant such as alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide (as described for example in WO92/06154, which is incorporated herein by reference).

A detergent composition herein typically comprises one or more detergent builders or builder systems. In some aspects, oxidized poly alpha-1,3-glucan can be included as a co-builder, in which it is used together with one or more additional builders such as any disclosed herein. Oxidized poly alpha-1,3-glucan compounds for use herein are disclosed in U.S. Patent Appl. Publ. No. 2015/0259439. In some embodiments incorporating at least one builder, the cleaning compositions comprise at least about 1%, from about 3% to about 60%, or even from about 5% to about 40%, builder by weight of the composition. Builders (in addition to oxidized poly alpha-1,3-glucan) include, but are not limited to, alkali metal, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1, 3, 5-trihydroxy benzene-2, 4, 6-trisulphonic acid, and carboxymethyloxysuccinic acid, various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Indeed, it is contemplated that any suitable builder will find use in various embodiments of the present disclosure. Additional examples of a detergent builder or complexing agent include zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl-or alkenylsuccinic acid, soluble silicates or layered silicates (e.g., SKS-6 from Hoechst).

In some embodiments, builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present disclosure, including those known in the art (See, e.g., EP2100949). In some embodiments, suitable builders can include phosphate builders and non-phosphate builders. In some embodiments, a builder is a phosphate builder. In some embodiments, a builder is a non-phosphate builder. A builder can be used in a level of from 0.1% to 80%, or from 5% to 60%, or from 10% to 50%, by weight of the composition. In some embodiments, the product comprises a mixture of phosphate and non-phosphate builders. Suitable phosphate builders include mono-phosphates, di-phosphates, tri-polyphosphates or oligomeric-polyphosphates, including the alkali metal salts of these compounds, including the sodium salts. In some embodiments, a builder can be sodium tripolyphosphate (STPP). Additionally, the composition can comprise carbonate and/or citrate, preferably citrate that helps to achieve a neutral pH composition. Other suitable non-phosphate builders include homopolymers and copolymers of polycarboxylic acids and their partially or completely neutralized salts, monomeric polycarboxylic acids and hydroxycarboxylic acids and their salts. In some embodiments, salts of the above mentioned compounds include ammonium and/or alkali metal salts, i.e., lithium, sodium, and potassium salts, including sodium salts. Suitable polycarboxylic acids include acyclic, alicyclic, hetero-cyclic and aromatic carboxylic acids, wherein in some embodiments, they can contain at least two carboxyl groups which are in each case separated from one another by, in some instances, no more than two carbon atoms.

A detergent composition herein can comprise at least one chelating agent. Suitable chelating agents include, but are not limited to copper, iron and/or manganese chelating agents and mixtures thereof. In embodiments in which at least one chelating agent is used, the composition comprises from about 0.1% to about 15%, or even from about 3.0% to about 10%, chelating agent by weight of the composition.

A detergent composition herein can comprise at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

A detergent composition herein can comprise one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. Additional dye transfer inhibiting agents include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents examples of which include ethylene-diamine-tetraacetic acid (EDTA); diethylene triamine penta methylene phosphonic acid (DTPMP); hydroxy-ethane diphosphonic acid (HEDP); ethylenediamine N,N′-disuccinic acid (EDDS); methyl glycine diacetic acid (MGDA); diethylene triamine penta acetic acid (DTPA); propylene diamine tetraacetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethyl ethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP) and derivatives thereof, which can be used alone or in combination with any of the above. In embodiments in which at least one dye transfer inhibiting agent is used, a composition herein may comprise from about 0.0001% to about 10%, from about 0.01% to about 5%, or even from about 0.1% to about 3%, by weight of the composition.

A detergent composition herein can comprise silicates. In some of these embodiments, sodium silicates (e.g., sodium disilicate, sodium metasilicate, and/or crystalline phyllosilicates) find use. In some embodiments, silicates are present at a level of from about 1% to about 20% by weight of the composition. In some embodiments, silicates are present at a level of from about 5% to about 15% by weight of the composition.

A detergent composition herein can comprise dispersants. Suitable water-soluble organic materials include, but are not limited to the homo-or co-polymeric acids or their salts, in which the polycarboxylic acid comprises at least two carboxyl radicals separated from each other by not more than two carbon atoms.

A detergent composition herein may additionally comprise one or more enzymes. Examples of enzymes include proteases, cellulases, hemicellulases, peroxidases, lipolytic enzymes (e.g., metallolipolytic enzymes), xylanases, lipases, phospholipases, esterases (e.g., arylesterase, polyesterase), perhydrolases, cutinases, pectinases, pectate lyases, mannanases, keratinases, reductases, oxidases (e.g., choline oxidase, phenoloxidase), phenoloxidases, lipoxygenases, ligninases, pullulanases, tannases, pentosanases, malanases, beta-glucanases, arabinosidases, hyaluronidases, chondroitinases, laccases, metalloproteinases, amadoriases, glucoamylases, alpha-amylases, beta-amylases, galactosidases, galactanases, catalases, carageenases, hyaluronidases, keratinases, lactases, ligninases, peroxidases, phosphatases, polygalacturonases, pullulanases, rhamnogalactouronases, tannases, transglutaminases, xyloglucanases, xylosidases, metalloproteases, arabinofuranosidases, phytases, isomerases, transferases and/or amylases in any combination.

In some embodiments, a detergent composition can comprise one or more enzymes (e.g., any disclosed herein), each at a level from about 0.00001% to about 10% by weight of the composition and the balance of cleaning adjunct materials by weight of composition. In some other embodiments, a detergent composition can also comprise each enzyme at a level of about 0.0001% to about 10%, about 0.001% to about 5%, about 0.001% to about 2%, or about 0.005% to about 0.5%, by weight of the composition.

Enzymes that may be comprised in a detergent composition herein may be stabilized using conventional stabilizing agents, e.g., a polyol such as propylene glycol or glycerol; a sugar or sugar alcohol; lactic acid; boric acid or a boric acid derivative (e.g., an aromatic borate ester).

A detergent composition in certain embodiments may comprise one or more other types of polymers in addition to a graft copolymer compound as disclosed herein. Examples of other types of polymers useful herein include carboxymethyl cellulose (CMC), dextran, poly (vinylpyrrolidone) (PVP), polyethylene glycol (PEG), poly (vinyl alcohol) (PVA), polycarboxylates such as polyacrylates, maleic/acrylic acid copolymers and lauryl methacrylate/acrylic acid copolymers.

A detergent composition herein may contain a bleaching system. For example, a bleaching system can comprise an H2O2 source such as perborate or percarbonate, which may be combined with a peracid-forming bleach activator such as tetraacetylethylenediamine (TAED) or nonanoyloxybenzenesulfonate (NOBS). Alternatively, a bleaching system may comprise peroxyacids (e.g., amide, imide, or sulfone type peroxyacids). Alternatively still, a bleaching system can be an enzymatic bleaching system comprising perhydrolase, for example, such as the system described in WO2005/056783.

A detergent composition herein may also contain conventional detergent ingredients such as fabric conditioners, clays, foam boosters, suds suppressors, anti-corrosion agents, soil-suspending agents, anti-soil redeposition agents, dyes, bactericides, tarnish inhibiters, optical brighteners, or perfumes. The pH of a detergent composition herein (measured in aqueous solution at use concentration) is usually neutral or alkaline (e.g., pH of about 7.0 to about 11.0).

It is believed that a graft copolymer herein can be included as an anti-redeposition agent and/or clay soil removal agent in a detergent composition such as a fabric care composition, if desired (such agents can optionally be characterized as whiteness maintenance agents in certain aspects). Examples of other suitable anti-redeposition and/or clay soil removal agents herein include polyethoxy zwitterionic surfactants, water-soluble copolymers of acrylic or methacrylic acid with acrylic or methacrylic acid-ethylene oxide condensates (e.g., U.S. Pat. No. 3,719,647), cellulose derivatives such as carboxymethylcellulose and hydroxypropylcellulose (e.g., U.S. Pat. Nos. 3,597,416 and 3,523,088), and mixtures comprising nonionic alkyl polyethoxy surfactant, polyethoxy alkyl quaternary cationic surfactant and fatty amide surfactant (e.g., U.S. Pat. No. 4,228,044). Non-limiting examples of other suitable anti-redeposition and clay soil removal agents are disclosed in U.S. Pat. Nos. 4,597,898 and 4,891,160, and Int. Patent Appl. Publ. No.

WO95/32272, all of which are incorporated herein by reference.

Particular forms of detergent compositions that can be adapted for purposes disclosed herein are disclosed in, for example, US20090209445A1, US20100081598A1, U.S. Pat. No. 7,001,878B2, EP1504994B1, WO2001085888A2, WO2003089562A1, WO2009098659A1, WO2009098660A1, WO2009112992A1, WO2009124160A1,

WO2009152031A1, WO2010059483A1, WO2010088112A1, WO2010090915A1, WO2010135238A1, WO2011094687A1, WO2011094690A1, WO2011127102A1, WO2011163428A1, WO2008000567A1, WO2006045391A1, WO2006007911A1, WO2012027404A1, EP1740690B1, WO2012059336A1, U.S. Pat. No. 6,730,646B1, WO2008087426A1, WO2010116139A1, and WO2012104613A1, all of which are incorporated herein by reference.

Laundry detergent compositions herein can optionally be heavy duty (all purpose) laundry detergent compositions. Exemplary heavy duty laundry detergent compositions comprise a detersive surfactant (10%- 40% wt/wt), including an anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof), and optionally non-ionic surfactant (selected from a group of linear or branched or random chain, substituted or unsubstituted alkyl alkoxylated alcohol, e.g., C8-C18 alkyl ethoxylated alcohols and/or C6-C12 alkyl phenol alkoxylates), where the weight ratio of anionic detersive surfactant (with a hydrophilic index (Hlc) of from 6.0 to 9) to non-ionic detersive surfactant is greater than 1:1. Suitable detersive surfactants also include cationic detersive surfactants (selected from a group of alkyl pyridinium compounds, alkyl quaternary ammonium compounds, alkyl quaternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

A detergent herein such as a heavy duty laundry detergent composition may optionally include, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt %-10 wt %) and/or random graft polymers (typically comprising of hydrophilic backbone comprising monomers selected from the group consisting of: unsaturated C1-C6 carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C4-C25 alkyl group, polypropylene, polybutylene, vinyl ester of a saturated C1-C6 mono-carboxylic acid, C1-C6 alkyl ester of acrylic or methacrylic acid, and mixtures thereof.

A detergent herein such as a heavy duty laundry detergent composition may optionally include additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example REPEL-O-TEX SF, SF-2 AND SRP6, TEXCARE SRA100, SRA300, SRN100, SRN170, SRN240, SRN300 AND SRN325, MARLOQUEST SL), anti-redeposition agent(s) herein (0.1 wt % to 10 wt %), include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); and polymeric carboxylate (such as maleate/acrylate random copolymer or polyacrylate homopolymer).

A detergent herein such as a heavy duty laundry detergent composition may optionally further include saturated or unsaturated fatty acids, preferably saturated or unsaturated C12-C24 fatty acids (0 wt % to 10 wt %); deposition aids (examples for which include polysaccharides, cellulosic polymers, poly diallyl dimethyl ammonium halides

(DADMAC), and co-polymers of DAD MAC with vinyl pyrrolidone, acrylamides, imidazoles, imidazolinium halides, and mixtures thereof, in random or block configuration, cationic guar gum, cationic starch, cationic polyacrylamides, and mixtures thereof.

A detergent herein such as a heavy duty laundry detergent composition may optionally further include dye transfer inhibiting agents, examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents, examples of which include ethylene-diamine-tetraacetic acid (EDTA), diethylene triamine penta methylene phosphonic acid (DTPMP), hydroxy-ethane diphosphonic acid (HEDP), ethylenediamine

N,N′-disuccinic acid (EDDS), methyl glycine diacetic acid (MGDA), diethylene triamine penta acetic acid (DTPA), propylene diamine tetraacetic acid (PDTA), 2-hydroxypyridine-N-oxide (HPNO), or methyl glycine diacetic acid (MGDA), glutamic acid N,N-diacetic acid (N,N-dicarboxymethyl glutamic acid tetrasodium salt (GLDA), nitrilotriacetic acid (NTA), 4,5-dihydroxy-m-benzenedisulfonic acid, citric acid and any salts thereof, N-hydroxyethylethylenediaminetriacetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), and derivatives thereof.

A detergent herein such as a heavy duty laundry detergent composition may optionally include silicone or fatty-acid based suds suppressors; hueing dyes, calcium and magnesium cations, visual signaling ingredients, anti-foam (0.001 wt % to about 4.0 wt %), and/or a structurant/thickener (0.01 wt % to 5 wt %) selected from the group consisting of diglycerides and triglycerides, ethylene glycol distearate, microcrystalline cellulose, microfiber cellulose, biopolymers, xanthan gum, gellan gum, and mixtures thereof). Such structurant/thickener would be, in certain embodiments, in addition to the one or more graft copolymers compounds comprised in the detergent. A structurant can also be referred to as a structural agent.

A detergent herein can be in the form of a heavy duty dry/solid laundry detergent composition, for example. Such a detergent may include: (i) a detersive surfactant, such as any anionic detersive surfactant disclosed herein, any non-ionic detersive surfactant disclosed herein, any cationic detersive surfactant disclosed herein, any zwitterionic and/or amphoteric detersive surfactant disclosed herein, any ampholytic surfactant, any semi-polar non-ionic surfactant, and mixtures thereof; (ii) a builder, such as any phosphate-free builder (e.g., zeolite builders in the range of 0 wt % to less than 10 wt %), any phosphate builder (e.g., sodium tri-polyphosphate in the range of 0 wt % to less than 10 wt %), citric acid, citrate salts and nitrilotriacetic acid, any silicate salt (e.g., sodium or potassium silicate or sodium meta-silicate in the range of 0 wt % to less than 10 wt %); any carbonate salt (e.g., sodium carbonate and/or sodium bicarbonate in the range of 0 wt % to less than 80 wt %), and mixtures thereof; (iii) a bleaching agent, such as any photobleach (e.g., sulfonated zinc phthalocyanines, sulfonated aluminum phthalocyanines, xanthenes dyes, and mixtures thereof), any hydrophobic or hydrophilic bleach activator (e.g., dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, nonanoyloxybenzene sulfonate-NOBS, nitrile quats, and mixtures thereof), any source of hydrogen peroxide (e.g., inorganic perhydrate salts, examples of which include mono or tetra hydrate sodium salt of perborate, percarbonate, persulfate, perphosphate, or persilicate), any preformed hydrophilic and/or hydrophobic peracids (e.g., percarboxylic acids and salts, percarbonic acids and salts, perimidic acids and salts, peroxymonosulfuric acids and salts, and mixtures thereof); and/or (iv) any other components such as a bleach catalyst (e.g., imine bleach boosters examples of which include iminium cations and polyions, iminium zwitterions, modified amines, modified amine oxides, N-sulphonyl imines, N-phosphonyl imines, N-acyl imines, thiadiazole dioxides, perfluoroimines, cyclic sugar ketones, and mixtures thereof), and a metal-containing bleach catalyst (e.g., copper, iron, titanium, ruthenium, tungsten, molybdenum, or manganese cations along with an auxiliary metal cations such as zinc or aluminum and a sequestrate such as EDTA, ethylenediaminetetra (methylenephosphonic acid).

Compositions disclosed herein can be in the form of a dishwashing detergent composition, for example. Examples of dishwashing detergents include automatic dishwashing detergents (typically used in dishwasher machines) and hand-washing dish detergents. A dishwashing detergent composition can be in any dry or liquid/aqueous form as disclosed herein, for example. Components that may be included in certain embodiments of a dishwashing detergent composition include, for example, one or more of a phosphate; oxygen-or chlorine-based bleaching agent; non-ionic surfactant; alkaline salt (e.g., metasilicates, alkali metal hydroxides, sodium carbonate); any active enzyme disclosed herein; anti-corrosion agent (e.g., sodium silicate); anti-foaming agent; additives to slow down the removal of glaze and patterns from ceramics; perfume; anti-caking agent (in granular detergent); starch (in tablet-based detergents); gelling agent (in liquid/gel based detergents); and/or sand (powdered detergents).

Dishwashing detergents such as an automatic dishwasher detergent or liquid dishwashing detergent can comprise (i) a non-ionic surfactant, including any ethoxylated non-ionic surfactant, alcohol alkoxylated surfactant, epoxy-capped poly (oxyalkylated) alcohol, or amine oxide surfactant present in an amount from 0 to 10 wt %; (ii) a builder, in the range of about 5-60 wt %, including any phosphate builder (e.g., mono-phosphates, di-phosphates, tri-polyphosphates, other oligomeric-polyphosphates, sodium tripolyphosphate-STPP), any phosphate-free builder (e.g., amino acid-based compounds including methyl-glycine-diacetic acid [MGDA] and salts or derivatives thereof, glutamic-N, N-diacetic acid [GLDA] and salts or derivatives thereof, iminodisuccinic acid (IDS) and salts or derivatives thereof, carboxy methyl inulin and salts or derivatives thereof, nitrilotriacetic acid [NTA], diethylene triamine penta acetic acid [DTPA], B-alaninediacetic acid [B-ADA] and salts thereof), homopolymers and copolymers of poly-carboxylic acids and partially or completely neutralized salts thereof, monomeric polycarboxylic acids and hydroxycarboxylic acids and salts thereof in the range of 0.5 wt % to 50 wt %, or sulfonated/carboxylated polymers in the range of about 0.1 wt % to about 50 wt %; (iii) a drying aid in the range of about 0.1 wt % to about 10 wt % (e.g., polyesters, especially anionic polyesters, optionally together with further monomers with 3 to 6 functionalities—typically acid, alcohol or ester functionalities which are conducive to polycondensation, polycarbonate-, polyurethane- and/or polyurea-polyorganosiloxane compounds or precursor compounds thereof, particularly of the reactive cyclic carbonate and urea type); (iv) a silicate in the range from about 1 wt % to about 20 wt % (e.g., sodium or potassium silicates such as sodium disilicate, sodium meta-silicate and crystalline phyllosilicates); (v) an inorganic bleach (e.g., perhydrate salts such as perborate, percarbonate, perphosphate, persulfate and persilicate salts) and/or an organic bleach (e.g., organic peroxyacids such as diacyl-and tetraacylperoxides, especially diperoxydodecanedioic acid, diperoxytetradecanedioic acid, and diperoxyhexadecanedioic acid); (vi) a bleach activator (e.g., organic peracid precursors in the range from about 0.1 wt % to about 10 wt %) and/or bleach catalyst (e.g., manganese triazacyclononane and related complexes; Co, Cu, Mn, and Fe bispyridylamine and related complexes; and pentamine acetate cobalt (III) and related complexes); (vii) a metal care agent in the range from about 0.1 wt % to 5 wt % (e.g., benzatriazoles, metal salts and complexes, and/or silicates); and/or (viii) any active enzyme disclosed herein in the range from about 0.01 to 5.0 mg of active enzyme per gram of automatic dishwashing detergent composition, and an enzyme stabilizer component (e.g., oligosaccharides, polysaccharides, and inorganic divalent metal salts).

Compositions disclosed herein can be in the form of an oral care composition, for example. Examples of oral care compositions include dentifrices, toothpaste, mouth wash, mouth rinse, chewing gum, and edible strips that provide some form of oral care (e.g., treatment or prevention of cavities [dental caries], gingivitis, plaque, tartar, and/or periodontal disease). An oral care composition can also be for treating an “oral surface”, which encompasses any soft or hard surface within the oral cavity including surfaces of the tongue, hard and soft palate, buccal mucosa, gums and dental surfaces. A “dental surface” herein is a surface of a natural tooth or a hard surface of artificial dentition including a crown, cap, filling, bridge, denture, or dental implant, for example.

An oral care composition herein can comprise about 0.01-15.0 wt % (e.g., ˜0.1-10 wt % or ˜0.1-5.0 wt %, ˜0.1-2.0 wt %) of one or more graft copolymer compounds as disclosed herein, for example. One or more graft copolymer compounds comprised in an oral care composition can sometimes be provided therein as a thickening agent and/or dispersion agent, which may be useful to impart a desired consistency and/or mouth feel to the composition. One or more other thickening or dispersion agents can also be provided in an oral care composition herein, such as a carboxyvinyl polymer, carrageenan (e.g., L-carrageenan), natural gum (e.g., karaya, xanthan, gum arabic, tragacanth), colloidal magnesium aluminum silicate, or colloidal silica, for example. An oral care composition herein may be a toothpaste or other dentifrice, for example. Such compositions, as well as any other oral care composition herein, can additionally comprise, without limitation, one or more of an anticaries agent, antimicrobial or antibacterial agent, anticalculus or tartar control agent, surfactant, abrasive, pH-modifying agent, foam modulator, humectant, flavorant, sweetener, pigment/colorant, whitening agent, and/or other suitable components. Examples of oral care compositions to which one or more graft copolymer compounds can be added are disclosed in U.S. Patent Appl. Publ. Nos. 2006/0134025, 2002/0022006 and 2008/0057007, which are incorporated herein by reference.

An anticaries agent herein can be an orally acceptable source of fluoride ions. Suitable sources of fluoride ions include fluoride, monofluorophosphate and fluorosilicate salts as well as amine fluorides, including olaflur (N′-octadecyltrimethylendiamine-N,N,N′-tris (2-ethanol)-dihydrofluoride), for example. An anticaries agent can be present in an amount providing a total of about 100-20000 ppm, about 200-5000 ppm, or about 500-2500 ppm, fluoride ions to the composition, for example. In oral care compositions in which sodium fluoride is the sole source of fluoride ions, an amount of about 0.01-5.0 wt %, about 0.05-1.0 wt %, or about 0.1-0.5 wt %, sodium fluoride can be present in the composition, for example.

An antimicrobial or antibacterial agent suitable for use in an oral care composition herein includes, for example, phenolic compounds (e.g., 4-allylcatechol; p-hydroxybenzoic acid esters such as benzylparaben, butylparaben, ethylparaben, methylparaben and propylparaben; 2-benzylphenol; butylated hydroxyanisole; butylated hydroxytoluene; capsaicin; carvacrol; creosol; eugenol; guaiacol; halogenated bisphenolics such as hexachlorophene and bromochlorophene; 4-hexylresorcinol; 8-hydroxyquinoline and salts thereof; salicylic acid esters such as menthyl salicylate, methyl salicylate and phenyl salicylate; phenol; pyrocatechol; salicylanilide; thymol; halogenated diphenylether compounds such as triclosan and triclosan monophosphate), copper (II) compounds (e.g., copper (II) chloride, fluoride, sulfate and hydroxide), zinc ion sources (e.g., zinc acetate, citrate, gluconate, glycinate, oxide, and sulfate), phthalic acid and salts thereof (e.g., magnesium monopotassium phthalate), hexetidine, octenidine, sanguinarine, benzalkonium chloride, domiphen bromide, alkylpyridinium chlorides (e.g. cetylpyridinium chloride, tetradecylpyridinium chloride, N-tetradecyl-4-ethylpyridinium chloride), iodine, sulfonamides, bisbiguanides (e.g., alexidine, chlorhexidine, chlorhexidine digluconate), piperidino derivatives (e.g., delmopinol, octapinol), magnolia extract, grapeseed extract, rosemary extract, menthol, geraniol, citral, eucalyptol, antibiotics (e.g., augmentin, amoxicillin, tetracycline, doxycycline, minocycline, metronidazole, neomycin, kanamycin, clindamycin), and/or any antibacterial agents disclosed in U.S. Pat. No. 5,776,435, which is incorporated herein by reference. One or more antimicrobial agents can optionally be present at about 0.01-10 wt % (e.g., 0.1-3 wt %), for example, in the disclosed oral care composition.

An anticalculus or tartar control agent suitable for use in an oral care composition herein includes, for example, phosphates and polyphosphates (e.g., pyrophosphates), polyaminopropanesulfonic acid (AMPS), zinc citrate trihydrate, polypeptides (e.g., polyaspartic and polyglutamic acids), polyolefin sulfonates, polyolefin phosphates, diphosphonates (e.g.,azacycloalkane-2,2-diphosphonates such as azacycloheptane-2,2-diphosphonic acid), N-methyl azacyclopentane-2,3-diphosphonic acid, ethane-1-hydroxy-1,1-diphosphonic acid (EHDP), ethane-1-amino-1,1-diphosphonate, and/or phosphonoalkane carboxylic acids and salts thereof (e.g., their alkali metal and ammonium salts). Useful inorganic phosphate and polyphosphate salts include, for example, monobasic, dibasic and tribasic sodium phosphates, sodium tripolyphosphate, tetrapolyphosphate, mono-, di-, tri-and tetra-sodium pyrophosphates, disodium dihydrogen pyrophosphate, sodium trimetaphosphate, sodium hexametaphosphate, or any of these in which sodium is replaced by potassium or ammonium. Other useful anticalculus agents in certain embodiments include anionic polycarboxylate polymers (e.g., polymers or copolymers of acrylic acid, methacrylic, and maleic anhydride such as polyvinyl methyl ether/maleic anhydride copolymers). Still other useful anticalculus agents include sequestering agents such as hydroxycarboxylic acids (e.g., citric, fumaric, malic, glutaric and oxalic acids and salts thereof) and aminopolycarboxylic acids (e.g., EDTA). One or more anticalculus or tartar control agents can optionally be present at about 0.01-50 wt % (e.g., about 0.05-25 wt % or about 0.1-15 wt %), for example, in the disclosed oral care composition.

A surfactant suitable for use in an oral care composition herein may be anionic, non-ionic, or amphoteric, for example. Suitable anionic surfactants include, without limitation, water-soluble salts of C₈₋₂₀ alkyl sulfates, sulfonated monoglycerides of C₈₋₂₀ fatty acids, sarcosinates, and taurates. Examples of anionic surfactants include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable non-ionic surfactants include, without limitation, poloxamers, polyoxyethylene sorbitan esters, fatty alcohol ethoxylates, alkylphenol ethoxylates, tertiary amine oxides, tertiary phosphine oxides, and dialkyl sulfoxides. Suitable amphoteric surfactants include, without limitation, derivatives of C₈₋₂₀ aliphatic secondary and tertiary amines having an anionic group such as a carboxylate, sulfate, sulfonate, phosphate or phosphonate. An example of a suitable amphoteric surfactant is cocoamidopropyl betaine. One or more surfactants are optionally present in a total amount of about 0.01-10 wt % (e.g., about 0.05-5.0 wt % or about 0.1-2.0 wt %), for example, in the disclosed oral care composition.

An abrasive suitable for use in an oral care composition herein may include, for example, silica (e.g., silica gel, hydrated silica, precipitated silica), alumina, insoluble phosphates, calcium carbonate, and resinous abrasives (e.g., a urea-formaldehyde condensation product). Examples of insoluble phosphates useful as abrasives herein are orthophosphates, polymetaphosphates and pyrophosphates, and include dicalcium orthophosphate dihydrate, calcium pyrophosphate, beta-calcium pyrophosphate, tricalcium phosphate, calcium polymetaphosphate and insoluble sodium polymetaphosphate. One or more abrasives are optionally present in a total amount of about 5-70 wt % (e.g., about 10-56 wt % or about 15-30 wt %), for example, in the disclosed oral care composition. The average particle size of an abrasive in certain embodiments is about 0.1-30 microns (e.g., about 1-20 microns or about 5-15 microns).

An oral care composition in certain embodiments may comprise at least one pH-modifying agent. Such agents may be selected to acidify, make more basic, or buffer the pH of a composition to a pH range of about 2-10 (e.g., pH ranging from about 2-8, 3-9, 4-8, 5-7, 6-10, or 7-9). Examples of pH-modifying agents useful herein include, without limitation, carboxylic, phosphoric and sulfonic acids; acid salts (e.g., monosodium citrate, disodium citrate, monosodium malate); alkali metal hydroxides (e.g. sodium hydroxide, carbonates such as sodium carbonate, bicarbonates, sesquicarbonates); borates; silicates; phosphates (e.g., monosodium phosphate, trisodium phosphate, pyrophosphate salts); and imidazole.

A foam modulator suitable for use in an oral care composition herein may be a polyethylene glycol (PEG), for example. High molecular weight PEGs are suitable, including those having an average molecular weight of about 200000-7000000 (e.g., about 500000-5000000 or about 1000000-2500000), for example. One or more PEGs are optionally present in a total amount of about 0.1-10 wt % (e.g. about 0.2-5.0 wt % or about 0.25-2.0 wt %), for example, in the disclosed oral care composition.

An oral care composition in certain embodiments may comprise at least one humectant. A humectant in certain embodiments may be a polyhydric alcohol such as glycerin, sorbitol, xylitol, or a low molecular weight PEG. Most suitable humectants also may function as a sweetener herein. One or more humectants are optionally present in a total amount of about 1.0-70 wt % (e.g., about 1.0-50 wt %, about 2-25 wt %, or about 5-15 wt %), for example, in the disclosed oral care composition.

A natural or artificial sweetener may optionally be comprised in an oral care composition herein. Examples of suitable sweeteners include dextrose, sucrose, maltose, dextrin, invert sugar, mannose, xylose, ribose, fructose, levulose, galactose, corn syrup (e.g., high fructose corn syrup or corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, and cyclamates. One or more sweeteners are optionally present in a total amount of about 0.005-5.0 wt %, for example, in the disclosed oral care composition.

A natural or artificial flavorant may optionally be comprised in an oral care composition herein. Examples of suitable flavorants include vanillin; sage; marjoram; parsley oil; spearmint oil; cinnamon oil; oil of wintergreen (methylsalicylate); peppermint oil; clove oil; bay oil; anise oil; eucalyptus oil; citrus oils; fruit oils; essences such as those derived from lemon, orange, lime, grapefruit, apricot, banana, grape, apple, strawberry, cherry, or pineapple; bean-and nut-derived flavors such as coffee, cocoa, cola, peanut, or almond; and adsorbed and encapsulated flavorants. Also encompassed within flavorants herein are ingredients that provide fragrance and/or other sensory effect in the mouth, including cooling or warming effects. Such ingredients include, without limitation, menthol, menthyl acetate, menthyl lactate, camphor, eucalyptus oil, eucalyptol, anethole, eugenol, cassia, oxanone, Irisone®, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), and menthone glycerol acetal (MGA). One or more flavorants are optionally present in a total amount of about 0.01-5.0 wt % (e.g., about 0.1-2.5 wt %), for example, in the disclosed oral care composition.

An oral care composition in certain embodiments may comprise at least one bicarbonate salt. Any orally acceptable bicarbonate can be used, including alkali metal bicarbonates such as sodium or potassium bicarbonate, and ammonium bicarbonate, for example. One or more bicarbonate salts are optionally present in a total amount of about 0.1-50 wt % (e.g., about 1-20 wt %), for example, in the disclosed oral care composition. An oral care composition in certain embodiments may comprise at least one whitening agent and/or colorant. A suitable whitening agent is a peroxide compound such as any of those disclosed in U.S. Pat. No. 8,540,971, which is incorporated herein by reference. Suitable colorants herein include pigments, dyes, lakes and agents imparting a particular luster or reflectivity such as pearling agents, for example. Specific examples of colorants useful herein include talc; mica; magnesium carbonate; calcium carbonate; magnesium silicate; magnesium aluminum silicate; silica; titanium dioxide; zinc oxide; red, yellow, brown and black iron oxides; ferric ammonium ferrocyanide; manganese violet; ultramarine; titaniated mica; and bismuth oxychloride. One or more colorants are optionally present in a total amount of about 0.001-20 wt % (e.g., about 0.01-10 wt % or about 0.1-5.0 wt %), for example, in the disclosed oral care composition.

Additional components that can optionally be included in an oral composition herein include one or more enzymes (above), vitamins, and anti-adhesion agents, for example. Examples of vitamins useful herein include vitamin C, vitamin E, vitamin B5, and folic acid. Examples of suitable anti-adhesion agents include solbrol, ficin, and quorum-sensing inhibitors.

The present disclosure also concerns a method of treating a material. This method comprises contacting a material with an aqueous composition comprising at least one graft copolymer compound as disclosed herein.

A material contacted with an aqueous composition in a contacting method herein can comprise a fabric in certain embodiments. A fabric herein can comprise natural fibers, synthetic fibers, semi-synthetic fibers, or any combination thereof. A semi-synthetic fiber herein is produced using naturally occurring material that has been chemically derivatized, an example of which is rayon. Non-limiting examples of fabric types herein include fabrics made of (i) cellulosic fibers such as cotton (e.g., broadcloth, canvas, chambray, chenille, chintz, corduroy, cretonne, damask, denim, flannel, gingham, jacquard, knit, matelassé, oxford, percale, poplin, plissé, sateen, seersucker, sheers, terry cloth, twill, velvet), rayon (e.g., viscose, modal, lyocell), linen, and Tencel®; (ii) proteinaceous fibers such as silk, wool and related mammalian fibers; (iii) synthetic fibers such as polyester, acrylic, nylon, and the like; (iv) long vegetable fibers from jute, flax, ramie, coir, kapok, sisal, henequen, abaca, hemp and sunn; and (v) any combination of a fabric of (i)-(iv). Fabric comprising a combination of fiber types (e.g., natural and synthetic) include those with both a cotton fiber and polyester, for example. Materials/articles containing one or more fabrics herein include, for example, clothing, curtains, drapes, upholstery, carpeting, bed linens, bath linens, tablecloths, sleeping bags, tents, car interiors, etc. Other materials comprising natural and/or synthetic fibers include, for example, non-woven fabrics, paddings, paper, and foams.

An aqueous composition that is contacted with a fabric can be, for example, a fabric care composition (e.g., laundry detergent, fabric softener). Thus, a treatment method in certain embodiments can be considered a fabric care method or laundry method if employing a fabric care composition therein. A fabric care composition herein is contemplated to effect one or more of the following fabric care benefits (i.e., surface substantive effects): wrinkle removal, wrinkle reduction, wrinkle resistance, fabric wear reduction, fabric wear resistance, fabric pilling reduction, extended fabric life, fabric color maintenance, fabric color fading reduction, reduced dye transfer, fabric color restoration, fabric soiling reduction, fabric soil release, fabric shape retention, fabric smoothness enhancement, anti-redeposition of soil on fabric, anti-greying of laundry, improved fabric hand/handle, and/or fabric shrinkage reduction.

Examples of conditions (e.g., time, temperature, wash/rinse volumes) for conducting a fabric care method or laundry method herein are disclosed in WO1997/003161 and U.S. Pat. Nos. 4,794,661, 4,580,421 and 5,945,394, which are incorporated herein by reference.

In other examples, a material comprising fabric can be contacted with an aqueous composition herein: (i) for at least about 5, 10, 20, 30, 40, 50, 60, 70, 80, 90, 100, 110, or 120 minutes; (ii) at a temperature of at least about 10, 15, 20, 25, 30, 35, 40, 45, 50, 55, 60, 65, 70, 75, 80, 85, 90, or 95° C. (e.g., for laundry wash or rinse: a “cold” temperature of about 15-30° C., a “warm” temperature of about 30-50° C., a “hot” temperature of about 50-95° C.); (iii) at a pH of about 2, 3, 4, 5, 6, 7, 8, 9, 10, 11, or 12 (e.g., pH range of about 2-12, or about 3-11); (iv) at a salt (e.g., NaCl) concentration of at least about 0.5, 1.0, 1.5, 2.0, 2.5, 3.0, 3.5, or 4.0 wt %; or any combination of (i)-(iv).

The contacting step in a fabric care method or laundry method can comprise any of washing, soaking, and/or rinsing steps, for example. Contacting a material or fabric in still further embodiments can be performed by any means known in the art, such as dissolving, mixing, shaking, spraying, treating, immersing, flushing, pouring on or in, combining, painting, coating, applying, affixing to, and/or communicating an effective amount of a graft copolymer compound herein with the fabric or material. In still further embodiments, contacting may be used to treat a fabric to provide a surface substantive effect. As used herein, the term “fabric hand” or “handle” refers to a person's tactile sensory response towards fabric which may be physical, physiological, psychological, social or any combination thereof. In one embodiment, the fabric hand may be measured using a

PhabrOmeter® System for measuring relative hand value (available from Nu Cybertek, Inc. Davis, CA) (American Association of Textile Chemists and Colorists [AATCC test method “202-2012, Relative Hand Value of Textiles: Instrumental Method”]).

In certain embodiments of treating a material comprising fabric, a graft copolymer compound component(s) of the aqueous composition adsorbs to the fabric. This feature is believed to render graft copolymer compounds herein useful as anti-redeposition agents and/or anti-greying agents in fabric care compositions disclosed (in addition to their viscosity-modifying effect). An anti-redeposition agent or anti-greying agent herein helps keep soil from redepositing onto clothing in wash water after the soil has been removed. It is further contemplated that adsorption of one or more graft copolymer compounds herein to a fabric enhances mechanical properties of the fabric.

Adsorption of a graft copolymer compound to a fabric herein can be measured using a colorimetric technique (e.g., Dubois et al., 1956, Anal. Chem. 28:350-356; Zemljič et al., 2006, Lenzinger Berichte 85:68-76; both incorporated herein by reference), for example, or any other method known in the art.

Other materials that can be contacted in the above treatment method include surfaces that can be treated with a dish detergent (e.g., automatic dishwashing detergent or hand dish detergent). Examples of such materials include surfaces of dishes, glasses, pots, pans, baking dishes, utensils and flatware made from ceramic material, china, metal, glass, plastic (e.g., polyethylene, polypropylene, polystyrene, etc.) and wood (collectively referred to herein as “tableware”). Thus, the treatment method in certain embodiments can be considered a dishwashing method or tableware washing method, for example. Examples of conditions (e.g., time, temperature, wash volume) for conducting a dishwashing or tableware washing method herein are disclosed in U.S. Pat. No. 8,575,083, which is incorporated herein by reference. In other examples, a tableware article can be contacted with an aqueous composition herein under a suitable set of conditions such as any of those disclosed above with regard to contacting a fabric-comprising material.

Other materials that can be contacted in the above treatment method include oral surfaces such as any soft or hard surface within the oral cavity including surfaces of the tongue, hard and soft palate, buccal mucosa, gums and dental surfaces (e.g., natural tooth or a hard surface of artificial dentition such as a crown, cap, filling, bridge, denture, or dental implant). Thus, a treatment method in certain embodiments can be considered an oral care method or dental care method, for example. Conditions (e.g., time, temperature) for contacting an oral surface with an aqueous composition herein should be suitable for the intended purpose of making such contact. Other surfaces that can be contacted in a treatment method also include a surface of the integumentary system such as skin, hair or nails.

Thus, certain embodiments of the present disclosure concern material (e.g., fabric) that comprises a graft copolymer compound herein. Such material can be produced following a material treatment method as disclosed herein, for example. A material may comprise a graft copolymer compound in certain embodiments if the compound is adsorbed to, or otherwise in contact with, the surface of the material.

Certain embodiments of a method of treating a material herein further comprise a drying step, in which a material is dried after being contacted with the aqueous composition. A drying step can be performed directly after the contacting step, or following one or more additional steps that might follow the contacting step (e.g., drying of a fabric after being rinsed, in water for example, following a wash in an aqueous composition herein). Drying can be performed by any of several means known in the art, such as air drying (e.g., ˜20-25° C.), or at a temperature of at least about 30, 40, 50, 60, 70, 80, 90, 100, 120, 140, 160, 170, 175, 180, or 200° C., for example. A material that has been dried herein typically has less than 3, 2, 1, 0.5, or 0.1 wt % water comprised therein. Fabric is a preferred material for conducting an optional drying step.

An aqueous composition used in a treatment method herein can be any aqueous composition disclosed herein, such as in the above embodiments or in the below Examples. Thus, the graft copolymer component(s) of an aqueous composition can be any as disclosed herein. Examples of aqueous compositions include detergents (e.g., laundry detergent or dish detergent) and water-containing dentifrices such as toothpaste.

Certain embodiments of the present disclosure concern a method of producing alpha-1,3-glucan, which method comprises: (a) contacting at least (i) water, (ii) sucrose, (iii) dextran that has been modified with about 1%- 50% alpha-1,2 branches, and (iv) a glucosyltransferase enzyme that synthesizes alpha-1,3-glucan comprising at least about 50% alpha-1,3 glycosidic linkages, thereby providing an enzymatic reaction, whereby alpha-1,3-glucan comprising at least about 50% alpha-1,3 glycosidic linkages is produced, and (b) optionally, isolating the alpha-1,3-glucan produced in step (a). This method is in addition to a method of producing a graft copolymer herein, which also represents a way of producing alpha-1,3-glucan. Significantly, step (a) of this a method can be used to modulate the molecular weight of the alpha-1,3-glucan product of the enzymatic reaction. In particular, using dextran with a lower percent alpha-1,2 branching results in production of alpha-1,3-glucan of higher/increased molecular weight compared to when using dextran with a higher percent alpha-1,2 branching (all other reaction conditions being identical or similar). For example, it is noted from the below Examples that reactions (˜100-110 g/L sucrose) comprising dextran with about 29.2%, 18.6%, 9.2%, or 0% alpha-1,2 branches produced, respectively, alpha-1,3-glucan graft copolymer with a DPw of about, on average, 1499 (Table 12), 1577 (Table 2), 2115 (Table 4), or 2790 (Table 10). In some aspects, the percent alpha-1,2 branching of dextran selected for use is decreased from about 30%, 25%, 20%, 15%, or 10% to about 20%, 15%, 10%, 5%, or 2.5%, as appropriate, to achieve an increase in molecular weight of graft copolymer product. In some aspects, an increase in molecular weight can be by about, or at least about, 5%, 10%, 20%, 30%, 40%, 50%, 60%, 70%, 80%, 90%, or 100%.

In some aspects, increasing the concentration of a dextran modified with alpha-1,2 branches results in production of alpha-1,3-glucan graft copolymer of lower molecular weight compared to when using dextran at a lower concentration (all other reaction conditions being identical or similar). This feature can be observed in the below Examples (e.g., see Tables 2, 4, 10, 15 and 16), for instance. In some aspects, a decrease in molecular weight can be by about, or at least about, 5%, 10%, 20%, 30%, 40%, 50%, 60%, or 70%.

Any of the foregoing features of a method herein for modulating (up or down) alpha-1,3-glucan product molecular weight can be as described elsewhere herein, such as above or in the below Examples. For example, the dextran used can be substantially linear dextran or completely linear dextran (prior to modification with about 1-50% alpha-1,2 branches). In some aspects, dextran with about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 1-31%, 1-30%, 9-31%, 9-30%, 10-31%, or 10-30% alpha-1,2 branches can be employed. In some aspects (e.g., lowering graft copolymer product molecular weight), the concentration of dextran used in a reaction herein can be increased from about 0.05, 0.1, 0.2, 0.5, 1.0, 2.0, or 2.5 g/L to about 0.5, 1.0, 2.0, 2.5, 5.0, or 10.0 g/L, as appropriate.

Non-limiting examples of compositions and methods disclosed herein include:
1. A composition comprising a graft copolymer, or an ether-or ester-derivative thereof, wherein the graft copolymer comprises: (i) a backbone comprising dextran that has been modified with about 1%- 25% alpha-1,2 branches, and (ii) one or more alpha-1,3-glucan side chains comprising at least about 50% alpha-1,3 glycosidic linkages.
2. The composition of embodiment 1, wherein the dextran is substantially linear dextran (e.g., completely linear dextran) that has been modified with about 1%- 25% alpha-1,2 branches.
3. The composition of embodiment 1 or 2, wherein the dextran has been modified with about 5%- 20% alpha-1,2 branches.
4. The composition of embodiment 1, 2, or 3, wherein one or more of the alpha-1,2 branches are a single glucose monomer in length.
5. The composition of embodiment 1, 2, 3, or 4, wherein the weight-average degree of polymerization (DPw) of the backbone is about 10 to 500.
6. The composition of embodiment 1, 2, 3, 4, or 5, wherein the alpha-1,3-glucan side chains comprise at least about 90% alpha-1,3 glycosidic linkages.
7. The composition of embodiment 1, 2, 3, 4, 5, or 6, wherein the DP or DPw of the one or more alpha-1,3-glucan side chains is at least about 10 or 100.
8. The composition of embodiment 1, 2, 3, 4, 5, 6, or 7, wherein the graft copolymer or ether-or ester-derivative thereof is insoluble under aqueous conditions, or wherein the graft copolymer or ether-or ester-derivative thereof is soluble under aqueous conditions.
9. The composition of embodiment 1, 2, 3, 4, 5, 6, 7, or 8, wherein the graft copolymer is produced in a reaction composition comprising at least water, sucrose, the dextran, and a glucosyltransferase enzyme that synthesizes alpha-1,3-glucan with at least about 50% alpha-1,3 glycosidic linkages.
10. The composition of embodiment 1, 2, 3, 4, 5, 6, 7, 8, or 9, wherein the composition is a household care product, personal care product, industrial product, pharmaceutical product, or food product.
11. The composition of embodiment 1, 2, 3, 4, 5, 6, 7, 8, 9, or 10, wherein the composition is: (a) a film or coating, optionally wherein the percent elongation at break of the film is at least about 20%, or (b) a detergent composition that optionally is a fabric care composition.
12. A method of producing a graft copolymer, the method comprising: (a) contacting at least (i) water, (ii) sucrose, (iii) dextran that has been modified with about 1%- 25% alpha-1,2 branches, and (iv) a glucosyltransferase enzyme that synthesizes alpha-1,3-glucan comprising at least about 50% alpha-1,3 glycosidic linkages, whereby a graft copolymer according to the composition of any one of embodiments 1-8 is produced; and (b) optionally, isolating the graft copolymer produced in step (a).
13. The method of embodiment 12, wherein the dextran is substantially linear dextran (e.g., completely linear dextran) that has been modified with about 1%- 25% alpha-1,2 branches.
14. The method of embodiment 12 or 13, wherein the dextran has been modified with about 5%- 20% alpha-1,2 branches.
15. The method of embodiment 12, 13, or 14, wherein the ratio, by weight, of the dextran to the sucrose is about 1:100 to about 1:5.
16. A method for producing a film, the method comprising: (a) dissolving the graft copolymer, or ether-or ester-derivative thereof, of any one of embodiments 1-8 (or an insoluble graft copolymer produced in a reaction composition according to embodiment 9) in a solvent to provide a solution; (b) contacting the solution with a surface; and (c) removing the solvent (e.g., by drying and/or coagulating the solution) to form a film.
17 The method of embodiment 16, wherein the solution of (a) comprises less than 15 wt % of the graft copolymer or ether-or ester-derivative thereof.
18. A method of producing alpha-1,3-glucan, the method comprising: (a) contacting at least (i) water, (ii) sucrose, (iii) dextran that has been modified with about 1%- 50% alpha-1,2 branches (e.g., substantially linear dextran, or completely linear dextran, that has been modified with about 1%- 50% alpha-1,2 branches), and (iv) a glucosyltransferase enzyme that synthesizes alpha-1,3-glucan comprising at least about 50% alpha-1,3 glycosidic linkages, thereby providing an enzymatic reaction, whereby alpha-1,3-glucan comprising at least about 50% alpha-1,3 glycosidic linkages is produced, and (b) optionally, isolating the alpha-1,3-glucan produced in step (a).

### EXAMPLES

The present disclosure is further exemplified in the following Examples. It should be understood that these Examples, while indicating certain aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### General Methods

### Analysis of Reaction Profiles

High-performance liquid chromatography (HPLC) samples were analyzed using an Agilent 1200 series instrument equipped with a refractive index (RI) detector. Analysis was performed on two separate columns: Bio-Rad AMINEX 87C (column 1) for separation of <DP3 sugars and Bio-Rad AMINEX 42A (column 2) for separation of DP2-DP7 and higher DP oligosaccharides (DP8+). Linear calibration curves were constructed for sucrose (0.1-50 g/L), fructose (0.1-100 g/L), leucrose (0.1-100 g/L), and glucose (0.1-100 g/L) for quantitation on column 1. Linear calibration curves were constructed for DP2 (0.04-10 g/L), DP3 (0.04-10 g/L), DP4 (0.04-10 g/L), DP5 (0.04-10 g/L), DP6 (0.04-10 g/L), DP7 (0.04-10 g/L), and DP8+ (0.25-10 g/L) oligosaccharides for quantitation on column 2. DP8+ oligosaccharide calibration was performed using P1 primer (described in Example 1 below). The yield of alpha-1,3-glucan, as synthesized in unprimed or primed reactions, was measured based on the glucosyl component of the reaction.

### Analysis of Glucan Molecular Weight

Insoluble glucan polymer wet cake isolated from glucosyltransferase reactions was treated with N,N-dimethylacetamide (DMAc) with 5% lithium chloride (LiCl) at 100° C. for 16 hours to form a glucan polymer solution. This solution (100 μL) was then injected into an Alliance™ 2695 HPLC (Waters Corporation, Milford, MA) equipped with a differential refractometer detector and multiangle light-scattering photometer from Wyatt Technologies (Santa Barbara, CA), both operating at 50° C. The mobile phase (DMAc containing 0.11 wt % LiCl) passed at a flow rate of 0.5 mL/min through four styrene-divinyl benzene columns in series; specifically, one KD-802, one KD-801, and two linear KD-806M columns (Shodex, Japan). The molecular weight distribution of the glucan polymer sample, as well as its average molar masses (Mn, Mw and Mz) were determined using Astra™ SEC-LS data reduction software package from Wyatt. Values of average degree of polymerization, e.g., DPw, were calculated by dividing a corresponding average molar mass by 162.

### Determination of Glycosidic Linkages

Glycosidic linkages in glucan products synthesized by a glucosyltransferase were determined by ¹H NMR (nuclear magnetic resonance) spectroscopy. Dry glucan polymer (6 to 8 mg) was dissolved in 0.75 mL of 3 wt % lithium chloride (LiCl) in deuterated dimethyl sulfoxide (DMSO-d6) by stirring overnight at ambient temperature. Deuterated water (D20) was then added (0.05 mL), and the sample was heated at 80° C. for about one hour to exchange protonated hydroxyls on the glucan polymer and ensure complete dissolution. 600 μL of the resulting clear homogeneous solution was transferred to a 5-mm NMR tube. ¹H NMR spectra was used to quantify glycosidic linkage and a 2D ¹H, ¹³C homo/hetero-nuclear suite of experiments was used to identify glucan linkages. The data were collected at 80° C. and processed on a Bruker Avance III NMR spectrometer, operating at either 500 MHz or 600 MHZ. The systems are equipped with a proton-optimized cryoprobe.

### Percent Primer Incorporation

The percent incorporation of dextran primer herein was calculated using the following formula: (g/L of primer consumed)/(g/L of starting primer)×100%.

### High Yielding Alpha-1,3-Glucan-Producing Glucosyltransferase Enzymes

The amino acid sequence of the glucosyltransferase used to prepare amino acid substitutions was SEQ ID NO:4 (GTF 6855), which essentially is an N-terminally truncated (signal peptide and variable region removed) version of the full-length wild type glucosyltransferase (represented by SEQ ID NO:62) from Streptococcus salivarius SK126 (see Table 1). Substitutions made in SEQ ID NO:4 can be characterized as substituting for native amino acid residues, as each amino acid residue/position of SEQ ID NO:4 (apart from the Met-1 residue of SEQ ID NO:4) corresponds accordingly with an amino acid residue/position within SEQ ID NO:62. In reactions comprising at least sucrose and water, the glucosyltransferase of SEQ ID NO:4 typically produces alpha-glucan having about 100% alpha-1,3 linkages and a DPw of 400 or greater (e.g., refer to U.S. Pat. Nos. 8,871,474 and 9,169,506, and U.S. Patent Appl. Publ. Nos. 2017/0002336 and 2017/0002335, which are incorporated herein by reference). This alpha-glucan product, which is insoluble, can be isolated following enzymatic synthesis via filtration, for example.

Briefly, certain combinations of amino acid substitutions were made to SEQ ID NO:4 (GTF 6855). These substitutions are listed in Tables A and B below. Each variant enzyme listed in Table A was entered into a glucan synthesis reaction with parameters that were the same as, or similar to, the following: vessel, 250-mL indented shake flask agitated at 120 rpm; initial pH, 5.7; reaction volume, 50 mL; sucrose, 75 g/L; GTF, 1.5 mL lysate of E. coli cells heterologously expressing enzyme; KH₂PO4, 20 mM; temperature, 30° C.; time, about 20-24 hours. The alpha-1,3-glucan yields of these reactions (measured by HPLC analysis) are provided in Table A.

TABLE A
Alpha-1,3-Glucan Yields of GTF 6855 (SEQ ID NO: 4)
Variants with Multiple Amino Acid Substitutions

| | Alpha-1,3- |
|---|---|
| GTF^{a} | Glucan^{b} Yield^{c} |
| A510D/F607Y/R741S | 72.6% |
| A510D/F607Y/N743S | 79.2% |
| A510D/F607Y/D948G | 88.2% |
| A510D/R741S/D948G | 74.5% |
| A510D/F607Y/R741S/D948G | 82.8% |
| A510E/F607Y/R741S/R1172C | 78.2% |
| A510D/F607Y/D820G/D948G | 87.8% |
| A510D/F607Y/D948G/R1172C | 88.6% |
| A510D/F607Y/N743S/D948G/R1172C | 89.4% |
| A510D/F607Y/R741S/L784Q/F929L/R1172C | 79.3% |

^{a}Each listed GTF is a version of GTF 6855 (SEQ ID NO: 4) comprising substitutions at respective positions, where each position number is in correspondence with the residue numbering of SEQ ID NO: 62. The wild type residue is listed first (before residue position number) and the substituting residue is listed second (after the residue position number).
^{b}Insoluble alpha-1,3-glucan product with 100% alpha-1,3 linkages.
^{c}Alpha-1,3-glucan yield based on glucosyl. The average yield of unmodified GTF 6855 (SEQ ID NO: 4, no substitutions) was about 29%.

Each variant enzyme listed in Table B was entered into a glucan synthesis reaction with parameters that were the same as, or similar to, the following: vessel, 500-mL jacketed reactor with Teflon®-pitched blade turbine (45-degree angle) on a glass stir rod and agitated at 50-200 rpm; initial pH, 5.5; reaction volume, 500 mL; sucrose, 108 g/L; KH₂PO₄, 1 mM; temperature, 39° C.; time, about 18-24 hours; filtrate from a previous alpha-1,3-glucan synthesis reaction, 50 vol %. The alpha-1,3-glucan yields of these reactions (measured by HPLC analysis) are provided in Table B.

TABLE B
Alpha-1,3 Glucan Yields of GTF 6855 (SEQ ID NO: 4) Variants with Multiple Amino Acid-Substitutions

| | | | | | | | | | | | | | Alpha-1,3- |
|---|---|---|---|---|---|---|---|---|---|---|---|---|---|
| | | | | | | | | | | | | | Glucan^{b} |
| GTF^{a} | | | | | | | | | | | | | Yield^{c} |
| A510D | Q588L | F607Y | R741S | D948G | | | R722H | T877K | | M1253I | K1277N | | 88% |
| A510D | Q588L | F607Y | R741S | D948G | | | R722H | T877K | V1188E | M1253I | Q957P | | 92% |
| A510D | Q588L | F607Y | R741S | D948G | | | | T877K | V1188E | M1253I | Q957P | | 91% |
| A510D | Q588L | F607Y | R741S | D948G | | | | | | M1253I | | | 89% |
| A510D | Q588L | F607W | R741S | D948G | | | | | | | | | 91% |
| | Q588L | F607Y | R741S | D948G | | | | | | | | | 91% |
| A510D | Q588L | F607Y | R741S | D948G | N628D | T635A | | T877K | | M1253I | F929L | R1172C | 92% |
| A510D | Q588L | F607W | R741S | D948G | S631T | S710G | R722H | T877K | V1188E | M1253I | | | 94% |
| A510D | Q588L | F607W | R741S | D948G | S631T | S710G | R722H | T877K | V1188E | | | | 93% |
| A510D | Q588L | F607W | R741S | D948G | S631T | S710G | | T877K | V1188E | M1253I | | | 96% |
| A510D | Q588L | F607Y | R741S | D948G | | | | | | | | | 89% |
| A510D | Q588L | F607Y | R741S | D948G | | | | | V1188E | | | | 88% |
| A510D | Q588L | F607W | R741S | D948G | S631T | S710G | | | V1188E | | | | 96% |
| A510D | Q588L | F607W | R741S | D948G | | S710G | R722H | T877K | | M1253I | | | 96% |
| A510D | Q588L | F607Y | R741S | D948G | S631T | | R722H | T877K | V1188E | M1253I | | | 96% |
| A510D | Q588L | F607W | R741S | D948G | S631T | | | T877K | V1188E | M1253I | | | 94% |
| A510D | Q588L | F607W | R741S | D948G | S631T | | | | V1188E | | | | 98% |
| A510D | Q588L | F607Y | R741S | D948G | S631T | | R722H | T877K | V1188E | M1253I | | | 95% |
| A510D | Q588L | F607W | R741S | D948G | | | | | V1188E | M1253I | | | 93% |

^{a}Each listed GTF is a version of GTF 6855 (SEQ ID NO: 4) comprising substitutions at respective positions, where each position number is in correspondence with the residue numbering of SEQ ID NO: 62.
^{b}Insoluble alpha-1,3 glucan product.
^{c}Alpha-1,3-glucan yield based on glucosyl.

Glucosyltransferase Enzymes that Produce Lower Molecular Weight Alpha-1,3-Glucan

As discussed above, SEQ ID NO:4 (GTF 6855) is an amino acid sequence of a glucosyltransferase that can be used to prepare amino acid substitutions. Briefly, certain combinations of amino acid substitutions can be made to SEQ ID NO:4 to provide a glucosyltransferase that produces alpha-1,3-glucan of lower molecular weight (as compared to alpha-1,3-glucan product of non-modified GTF 6855). These substitutions are listed in Tables C and D below. For collecting the data in these tables, each variant enzyme was entered into a glucan synthesis reaction with parameters that were the same as, or similar to, the following: vessel, 50-mL indented shake flask agitated at 75 rpm; initial pH, 5.7; reaction volume, 10 mL; sucrose, 400 g/L; GTF, 0.3 mL of culture supernatant (prepared from lysate of E. coli cells heterologously expressing enzyme); KH₂PO4, 5 mM; temperature, 35° C.; time, about two days; de-activation, heated at 80° C. for 30 minutes. Insoluble glucan polymers produced in the reactions were individually harvested, water-washed, and analyzed for molecular size (DPw) via a standard SEC approach (see Tables C and D for DPw data). Glucosyltransferases with any of the amino acid substitution(s) listed in Tables C and D should be useful in practicing the presently disclosed subject matter.

TABLE C
DPw of Insoluble Alpha-1,3-Glucan Produced by GTF 6855 (SEQ
ID NO: 4) and Single Amino Acid-Substituted Variants thereof

| GTF | DPw | GTF | DPw | GTF | DPw |
|---|---|---|---|---|---|
| 6855^{a} | 350 | S553A | 127 | N573A | 123 |
| L513A^{b} | 194 | S553C | 125 | N573A | 125 |
| L513C | 119 | S553C | 126 | N573D | 108 |
| L513C | 159 | S553E | 105 | N573D | 134 |
| L513D | 147 | S553E | 122 | N573G | 126 |
| L513D | 640 | S553F^{c} | | N573G | 120 |
| L513E | 129 | S553F^{c} | | N573H | 148 |
| L513E | 130 | S553H^{c} | | N573I^{c} | |
| L513F | 171 | S553H^{c} | | N573K | 145 |
| L513G | 138 | S553I | 79 | N573K | 148 |
| L513H | 153 | S553I | 97 | N573L^{c} | |
| L513H | 175 | S553M | 129 | N573M^{c} | |
| L513I | 186 | S553M | 140 | N573N | 222 |
| L513K | 143 | S553N | 77 | N573P | 100 |
| L513K | 160 | S553N | 69 | N573T | 102 |
| L513M | 183 | S553R | 63 | N573T | 109 |
| L513M | 210 | S553T | 226 | N573V | 91 |
| L513N^{c} | | S553T | 124 | N573W | 249 |
| L513N | 372 | S553V | 86 | N573W | 237 |
| L513P | 173 | S553Y | 110 | | |
| L513Q | 138 | S553Y | 52 | | |
| L513Q | 152 | | | | |
| L513R | 134 | | | | |
| L513R | 141 | | | | |
| L513S | 138 | | | | |
| L513S | 152 | | | | |
| L513T | 146 | | | | |
| L513V | 175 | | | | |
| L513V^{c} | | | | | |
| L513W | 146 | | | | |
| L513W | 171 | | | | |
| L513Y | 156 | | | | |
| GTF | DPw | GTF | DPw | GTF | DPw |
| 6855^{a} | 350 | K578A | 110 | Q616A | 175 |
| D575A^{b} | 74 | K578A | 113 | Q616A | 440 |
| D575A | 199 | K578C | 132 | Q616C | 81 |
| D575C | 97 | K578D | 156 | Q616D | 115 |
| D575C^{c} | | K578E | 95 | Q616E | 50 |
| D575C | 94 | K578E^{c} | | Q616G | 66 |
| D575E | 90 | K578F | 103 | Q616G^{c} | |
| D575E | 88 | K578G | 113 | Q616H | 61 |
| D575F | 74 | K578G | 103 | Q616I | 82 |
| D575G | 90 | K578H | 212 | Q616K | 58 |
| D575G | 89 | K578H | 187 | Q616K | 59 |
| D575H | 70 | K578I | 179 | Q616L | 61 |
| D575H | 134 | K578L | 177 | Q616L | 62 |
| D575I | 76 | K578M | 135 | Q616M | 164 |
| D575I | 98 | K578M | 141 | Q616N | 269 |
| D575K | 52 | K578N | 185 | Q616N | 211 |
| D575K | 95 | K578P | 126 | Q616P | 75 |
| D575L | 74 | K578P | 128 | Q616P | 78 |
| D575L^{c} | | K578Q | 111 | Q616R | 103 |
| D575M | 66 | K578R | 214 | Q616R | 167 |
| D575M | 72 | K578R | 294 | Q616S | 72 |
| D575N | 90 | K578S | 105 | Q616T | 79 |
| D575N | 191 | K578S | 105 | Q616V | 88 |
| D575N^{c} | | K578T | 131 | Q616V | 97 |
| D575P | 50 | K578T | 157 | Q616W | 60 |
| D575R | 65 | K578V | 146 | Q616W | 101 |
| D575R | 71 | K578V | 145 | Q616Y | 65 |
| D575S | 104 | K578W | 106 | | |
| D575S | 96 | K578W | 122 | | |
| D575V | 54 | K578Y | 145 | | |
| D575W | 69 | | | | |
| D575W | 167 | | | | |
| D575Y | 124 | | | | |
| D575Y | 69 | | | | |

^{a}GTF 6855, SEQ ID NO: 4. The DPw of insoluble alpha-1,3-glucan produced by GTF 6855 averaged to be about 350.
^{b}Each listed GTF with a substitution is a version of GTF 6855 comprising a substitution at a respective position, where the position number is in correspondence with the residue numbering of SEQ ID NO: 62.
^{c}Insoluble alpha-1,3-glucan not produced or detected.

TABLE D
DPw of Insoluble Alpha-1,3-Glucan Produced by Multiple Amino
Acid-Substituted Variants of GTF 6855 (SEQ ID NO: 4)

| GTF^{a} | | | | | | DPw |
|---|---|---|---|---|---|---|
| P550L | N557I | N581P | | | | 12 |
| L535P | S553C | N558D | D575V | T585P | K697R | 12 |
| P550V | S553R | N581P | T585P | | | 12 |
| P550L | S553F | N581P | | | | 12 |
| P550V | N557E | T585P | | | | 12 |
| P550L | N557E | D575V | T585P | | | 13 |
| L538P | P550L | S553Y | | | | 13 |
| P544L | P550V | S553C | N573I | T585P | S589G | 13 |
| P550V | G576D | T585P | | | | 13 |
| P550L | N558D | T585P | T679I | | | 13 |
| P550L | N557E | T585P | S589G | | | 14 |
| P550L | N557E | T569L | N581P | | | 14 |
| P550L | N557E | T569L | T585P | | | 14 |
| P550V | S553T | N558D | T585P | G730D | | 14 |
| E577G | P550L | N557I | T569L | N573I | | 14 |
| P550L | S553C | D575A | T585P | S589G | | 14 |
| S553R | N573V | K578N | S631G | T660A | | 14 |
| P550V | S553R | W571V | G576D | | | 14 |
| P550V | N557E | K578D | T585P | | | 14 |
| P550V | N558D | N573P | T585P | | | 14 |
| P550L | N558D | W571V | N581P | K593E | | 14 |
| P550V | S553E | N581P | | | | 15 |
| P550L | N573I | T585P | W725R | | | 15 |
| P550L | N557I | N573P | | | | 15 |
| N557E | N573V | N581P | | | | 15 |
| P550L | N557I | G576D | Q643L | | | 15 |
| P550V | S553N | T585P | V586G | S710G | | 15 |
| P550V | S553C | D575A | T585P | | | 15 |
| S553R | N573V | K578N | S631G | T660A | | 15 |
| P550L | S553K | D575A | Y580H | | | 16 |
| P550V | D575A | T585P | S589G | K713E | | 16 |
| P550V | S553N | N573I | Y693C | | | 16 |
| P544L | P550L | N557E | N573I | T585P | | 16 |
| P550L | N558D | W571V | N581P | T585P | | 16 |
| S504G | P550V | N557Q | N581P | | | 16 |
| P550L | S553R | D575A | | | | 16 |
| P550V | N558D | W571D | D575A | T585P | | 16 |
| P544L | P550V | N557Q | N581P | | | 17 |
| P550V | S553K | T585P | | | | 17 |
| P550V | S553N | T585P | | | | 17 |
| P550L | T569L | N573I | | | | 17 |
| P550L | N558D | D575V | | | | 17 |
| L537P | P550L | N558D | N573I | | | 17 |
| P550L | S553C | W571C | G576D | T585P | | 17 |
| P550L | N557Q | W571C | G576D | T585P | | 17 |
| P550L | N558D | W571V | N581P | | | 17 |
| A542V | P550V | N558D | W571V | T585P | | 17 |
| P550V | N558D | W571D | G576D | | | 18 |
| P550V | S553N | N573I | | | | 18 |
| P550V | N557Q | D575V | A669T | | | 18 |
| P550V | N581P | I636T | | | | 18 |
| P550V | N557E | N581P | | | | 18 |
| P550V | N573I | T585P | | | | 18 |
| P550L | S553K | N558D | K578R | Y700N | | 19 |
| P550V | S553T | N558D | W571V | | | 19 |
| P514L | P550V | N557Q | T585P | D602N | | 20 |
| P550L | N557I | T569A | G576D | | | 20 |
| P550V | N557T | N558D | W571D | | | 21 |
| P550L | N557E | D575A | T585P | | | 21 |
| I545V | P550V | N557Q | T585P | D638N | | 21 |
| P544L | P550V | N557I | T585P | | | 22 |
| Y518C | P550V | N581P | T585P | | | 22 |
| P550L | N557E | D575A | | | | 22 |

^{a}Each listed GTF is a version of GTF 6855 (SEQ ID NO: 4) comprising substitutions at respective positions, where each position number is in correspondence with the residue numbering of SEQ ID NO: 62.

Glucosyltransferase Enzymes that Produce Higher Molecular Weight Alpha-1,3-Glucan

As discussed above, SEQ ID NO:4 (GTF 6855) is an amino acid sequence of a glucosyltransferase that can be used to prepare amino acid substitutions. Briefly, certain amino acid substitutions can be made to SEQ ID NO:4 to provide a glucosyltransferase that produces alpha-1,3-glucan of higher molecular weight (as compared to alpha-1,3-glucan product of non-modified GTF 6855). Some of these substitutions are listed in Tables E and F below, along with mutations that did not have a substantial effect on molecular weight. For collecting the data in these tables, each variant enzyme was entered into a glucan synthesis reaction with parameters as disclosed in U.S. patent application Ser. No. 16/127,288, which is incorporated herein by reference. A glucosyltransferase with any of the amino acid substitution(s) listed in Tables E and F is contemplated to be useful in practicing the presently disclosed subject matter.

TABLE E
DPw of Insoluble Alpha-1,3-Glucan Produced by GTF 6855
(SEQ ID NO: 4) and Single Amino Acid-Substituted Variants thereof

| GTF | DPw | GTF | DPw | GTF | DPw | GTF | DPw |
|---|---|---|---|---|---|---|---|
| 6855^{a} | 626 | T635H | 539 | P1499Y | 587 | A510E | 625 |
| V186A^{b} | 589 | T635W | 528 | Y219C | 591 | N904E | 554 |
| V186M | 580 | I636H | 521 | E243H | 631 | K930G | 637 |
| E194C | 580 | Y848E | 843 | A377I | 514 | K930V | 582 |
| L434N | 613 | D947G | 408 | I411F | 586 | D947F | 619 |
| A472C | 530 | F951Y | 325 | I411S | 591 | D947I | 610 |
| A472S | 374 | E849M | 610 | D425Q | 681 | D947K | 559 |
| A510E | 654 | Q1007A | 394 | L428V | 577 | D947N | 635 |
| A510I | 621 | D1003G | 486 | M529N | 560 | D947Q | 635 |
| A510V | 655 | A1022M | 303 | N531G | 977 | D947S | 603 |
| M529L | 558 | D1028L | 416 | G576R | 416 | D947V | 621 |
| R534G | 711 | D1028Q | 537 | Y580H | 554 | D947Y | 624 |
| R534I | 789 | A1057H | 624 | K593M | 792 | Q1007S | 578 |
| R534L | 763 | N1096A | 562 | I608Y | 708 | D1003N | 570 |
| R534M | 776 | Y1104M | 611 | N613G | 644 | I1026H | 621 |
| G576H | 436 | N1122K | 614 | N613L | 618 | D1028A | 568 |
| Q588L | 817 | E1132A | 589 | D617E | 419 | D1028M | 535 |
| I591K | 816 | E1132H | 611 | E621T | 603 | V1037A | 591 |
| I591R | 832 | E1132K | 610 | I627W | 506 | K1041A | 583 |
| Y605W | 524 | E1132R | 622 | S631D | 521 | K1041M | 648 |
| F607N | 561 | V1135K | 612 | S631E | 545 | D1080M | 554 |
| F607W | 624 | V1188E | 641 | S631R | 521 | F1244P | 589 |
| A610C | 799 | L1212N | 630 | G633W | 493 | F1244Q | 534 |
| N613I | 555 | E1250R | 606 | F634A | 523 | E1250H | 553 |
| N613M | 587 | T1381E | 612 | T635E | 561 | E1250K | 591 |
| N613T | 526 | T1431M | 625 | T635I | 648 | T1431Q | 663 |
| N613V | 578 | A1442R | 609 | T635Y | 518 | E1450D | 585 |
| K625A | 638 | E1450F | 611 | R722H | 793 | G1484P | 627 |
| K625M | 623 | E1450W | 618 | T728S | 769 | I1453G | 881 |
| A510E | 622 | I1453M | 635 | M732L | 791 | W1437N | 654 |
| S631T | 532 | V1491F | 604 | A777N | 755 | R722N | 766 |

^{a}GTF 6855, SEQ ID NO: 4. The DPw of insoluble alpha-1,3-glucan produced by GTF 6855 averaged to be about 626.
^{b}Each listed GTF with a substitution is a version of GTF 6855 comprising a substitution at a respective position, where the position number is in correspondence with the residue numbering of SEQ ID NO: 62.

TABLE F
DPw of Insoluble Alpha-1,3-Glucan Produced by GTF 6855 (SEQ
ID NO: 4) and Single Amino Acid-Substituted Variants thereof

| GTF | DPw |
|---|---|
| 6855^{a} | 558 |
| E567Q^{b} | 1001 |
| I591V | 859 |
| L661P | 842 |
| N743D | 700 |
| N743S | 937 |
| N743T | 874 |
| R741A | 831 |
| R741P | 871 |
| R741Q | 886 |
| R741S | 887 |
| R741T | 693 |
| T563A | 910 |
| V586T | 874 |

^{a}GTF 6855, SEQ ID NO: 4.
^{b}Each listed GTF with a substitution is a version of GTF 6855 comprising a substitution at a respective position, where the position number is in correspondence with the residue numbering of SEQ ID NO: 62.

### EXAMPLE 1

### Producing Alpha-1,3-Glucan in Glucosyltransferase Reactions Containing Dextran with 18.6% Alpha-1,2 Branches

This Example describes priming alpha-1,3-glucan synthesis reactions with dextran that was previously modified to have 18.6% alpha-1,2 branches. Graft copolymers were synthesized comprising a dextran backbone and alpha-1,3-glucan arms.

Alpha-1,2-branched dextran was produced to prime glucosyltransferase reactions for alpha-1,3-glucan synthesis. Preparation of this primer was performed essentially as described in International Patent. Appl. Publ. No. WO2017/091533 and U.S. Patent Appl. Publ. No. 2018/0282385, which are both incorporated herein by reference. Briefly, linear dextran (i.e., polysaccharide with 100% alpha-1,6 glycosidic linkages) of molecular weight ˜14 kDa (DPw of ˜89) was produced using glucosyltransferase GTF8117 (SEQ ID NO:66). This dextran was then modified to have 18.6% alpha-1,2-branches using alpha-1,2-branching enzyme GTFJ18T1 (SEQ ID NO:70). The resulting alpha-1,2-branched dextran, which had a DPw of about 110 (˜17 kDa), is referred to herein as P1 primer.

P1 primer was then included in alpha-1,3-glucan synthesis reactions comprising a glucosyltransferase to produce graft copolymers comprising a dextran backbone and alpha-1,3-glucan arms. The glucosyltransferase used in these reactions, which produces alpha-1,3-glucan with about 100% alpha-1,3 linkages, was a Streptococcus salivarius glucosyltransferase modified in its catalytic domain such that the enzyme could produce more products (alpha-1,3-glucan and fructose), and less by-products (e.g., glucose, oligosaccharides such as leucrose and DP2-7 gluco-oligosaccharides), from sucrose substrate, as compared to the enzyme's unmodified counterpart. The General Methods section describes preparation of this non-native glucosyltransferase (Table A).

Eight separate 100-mL alpha-1,3-glucan synthesis reactions were performed in plastic 250-mL shake flasks. Each reaction comprised water, sucrose (roughly 50 or 100 g/L), phosphate buffer (5 mM), P1 primer (roughly 0-10 g/L), and a glucosyltransferase (above). The reactions were prepared by mixing 10 or 20 mL of a 500 g/L sucrose stock solution with 5 mL of a 100 mM sodium phosphate buffer stock (adjusted to pH 5.5 with a few drops of NaOH or phosphoric acid solution). A 25 g/L P1 primer stock was used to achieve a final concentration of 0, 1, 2.5, or 10 g/L by adding 0, 4, 10 or 40 mL to each preparation containing 50 or 100 g/L sucrose to yield a total of eight separate reaction preparations. Deionized, sub-micron filtered water was added to bring the final volume to 100 mL. A 0.5-mL aliquot was withdrawn from each preparation, after which 0.2256 mL of glucosyltransferase enzyme stock was added (for final enzyme concentration of 100 U/L) to initiate each reaction for alpha-1,3-glucan synthesis. The reactions were carried out at 30° C.

At 1, 2, 3 and 24 hours after commencing the reactions, 1 mL of each reaction was withdrawn to a micro-centrifuge tube. Samples were heated to 80° C. for ˜10 minutes in a dry heat block to deactivate the glucosyltransferase to stop the reaction. Deactivated samples were then spun in a table centrifuge for 10 minutes at 12,000 RPM to pellet the water-insoluble polymer products. The supernatant was withdrawn and filtered through a 0.45-μm WHATMAN syringeless filter for HPLC analysis. After 24 hours, all the reactions were placed into an 80° C. water bath for ˜30 minutes to deactivate the glucosyltransferase to stop the reaction. The deactivated reactions were charged on a ˜10-μm CHEMRUS fritted filter and vacuum-filtered until near-dry before washing twice with ˜100 ml of cold deionized water to form a polymer wet cake. Approximately 300-500 mg of each wet cake was reserved for size-exclusion chromatography (SEC) analysis, while the remainder of each wet cake was dried in a vacuum oven at 40° C., −40 cmHg.

SEC analysis was performed to determine the molecular weight and degree of polymerization (DP) of the insoluble polymer product. PDI (polydispersity index) values were calculated using these data. The glycosidic linkage profile of each insoluble polymer product was determined by ¹H NMR analysis using dried polymer dissolved in 3% LiCl/DMSO-d₆ containing a small amount of D2O. Table 2 provides the results of these analyses.

TABLE 2
Structural Analysis of Alpha-1,3-Glucan Produced in
Glucosyltransferase Reactions (24-hour) Containing P1 Primer

| | | Alpha-1,3-Glucan Product Profile | | | | | | |
|---|---|---|---|---|---|---|---|---|
| Reaction | | | | | Linkages | | | |
| Sucrose | Primer | | | % | (% of total residues) | | | |
| (g/L) | (g/L) | DPw | PDI | primer | 1,3 | 1,2,6 | 1,2 | 1,6 |
| 53 | 0.0 | 860.0 | 1.66 | 0.0 | 100.0 | 0.0 | 0.0 | 0.0 |
| | 1.1 | 2434.7 | 1.9 | 2.8 | 97.2 | 0.3 | 0.4 | 2.2 |
| | 2.6 | 1987.4 | 1.6 | 4.4 | 95.6 | 0.6 | 0.6 | 3.2 |
| | 10.5 | 2081.1 | 2.0 | 6.2 | 93.8 | 0.7 | 0.8 | 4.6 |
| 105 | 0.0 | 792.0 | 1.7 | 0.0 | 100.0 | 0.0 | 0.0 | 0.0 |
| | 1.0 | 1666.6 | 2.0 | 1.6 | 98.4 | 0.2 | 0.2 | 1.2 |
| | 2.6 | 1495.2 | 1.8 | 2.7 | 97.3 | 0.3 | 0.3 | 2.0 |
| | 10.4 | 1572.6 | 2.0 | 3.7 | 96.3 | 0.4 | 0.5 | 2.8 |

HPLC data were used to estimate the rates of the glucosyltransferase reactions based on sucrose consumption, glucan polymer yield (based on glucosyl [throughout the Examples]), and primer consumption. Monitoring the consumption of primer allowed for estimating primer incorporation into glucan polymer products. The size of individual alpha-1,3-glucan arms grafted on primer in each reaction was expected to be very similar to the size of alpha-1,3-glucan produced in control reactions that did not contain added primer; the control reaction products comprised alpha-1,3-glucan homopolymer. A glucan-to-primer molar ratio (M ratio) was calculated at 3-hour and 24-hour reaction times using this control DPw value (860 or 792) and the DPw of the P1 primer (˜110). The data from these analyses are provided in Table 3. Differences in the glucan-to-primer molar ratios between the 3-and 24-hour timepoints indicated the consistency of the priming reactions.

TABLE 3
Profiles of Glucosyltransferase Reactions
(24-hour) Containing P1 Primer

| | | Alpha-1,3- | | Glucan/ | Glucan/ |
|---|---|---|---|---|---|
| | | Glucan | Sucrose | Primer | Primer |
| Sucrose | Primer | Yield | Rate | 3 hour | 24 hour |
| (g/L) | (g/L) | (Glucosyl) | (g/L/h) | (M ratio)^{a} | (M ratio)^{a} |
| 53 | 0.0 | 79% | 7.5 | NA | NA |
| | 1.1 | 77% | 7.3 | 2.2 | 3.5 |
| | 2.6 | 78% | 8.2 | 2.2 | 2.6 |
| | 10.5 | 82% | 9.2 | 3.2 | 2.2 |
| 105 | 0.0 | 82% | 10.8 | NA | NA |
| | 1.0 | 79% | 10.1 | 3.2 | 7.3 |
| | 2.6 | 81% | 11.0 | 2.5 | 4.5 |
| | 10.4 | 82% | 12.5 | 2.6 | 5.5 |

^{a}Glucan/primer molar ratio was calculated using DPw (estimated) of glucan arm (860) for 53 g/L sucrose reactions, 792 for 105 g/L sucrose reactions) and DPw of primer (110).

Thus, by providing an alpha-1,2-branched dextran primer to alpha-1,3-glucan synthesis reactions, graft copolymers were synthesized comprising a dextran backbone and alpha-1,3-glucan arms. While primer was demonstrably consumed and incorporated into graft copolymer products (based on HPLC and NMR results), changes in reaction rate seemed minimal. It seemed that a higher primer concentration resulted in higher primer incorporation and higher glucan yield. Molecular weight increases were observed when primer was present (especially at 1 g/L); this was a sign of generating primed glucan product. The effect of priming on DPw and primer consumption was less in reactions with 100 g/L sucrose than those with 50 g/L sucrose, possibly indicating that less priming occurs as more sucrose is consumed. The graft copolymer products likely had an average of one to two alpha-1,3-glucan arms per dextran backbone.

### EXAMPLE 2

### Producing Alpha-1,3-Glucan in Glucosyltransferase Reactions Containing Dextran with 9.2% Alpha-1.2 Branches

This Example describes priming alpha-1,3-glucan synthesis reactions with dextran that was previously modified to have 9.2% alpha-1,2 branches. Graft copolymers were synthesized comprising a dextran backbone and alpha-1,3-glucan arms. In view of the results provided in Example 1, this Example indicates that alpha-1,3-glucan priming efficacy can be controlled by varying the alpha-1,2 branching frequency of a dextran primer.

Alpha-1,2-branched dextran was produced to prime glucosyltransferase reactions for alpha-1,3-glucan synthesis. Preparation of this primer was performed as described in Example 1 following the procedures disclosed in International Patent. Appl. Publ. No. WO2017/091533 and U.S. Patent Appl. Publ. No. 2018/0282385, with the exception that alpha-1,2-branching enzyme GTF9905 (SEQ ID NO:71) was used (instead of GTFJ18T1) with a different amount of sucrose, and the level of alpha-1,2 branches in the primer was 9.2% (instead of 18.6%). This primer, which had a molecular weight of ˜15 kDa (DPw ˜90), is referred to herein as P2 primer. Thus, the only apparent difference between the P1 and P2 primers is their respective levels of alpha-1,2 branching.

P2 primer was then included in alpha-1,3-glucan synthesis reactions comprising a glucosyltransferase (the same enzyme as used in Example 1) to produce graft copolymers comprising a dextran backbone and alpha-1,3-glucan arms. Briefly, eight plastic shake flasks were charged with 10 or 20 mL of a 500 g/L sterile-filtered sucrose stock solution, 5 mL of a 100 mM sodium phosphate buffer stock (adjusted to pH 5.5 with a few drops of NaOH or phosphoric acid solution), and 0, 0.48, 1.20, or 4.81 mL of a 208.1 g/L P2 primer stock. Deionized, sub-micron filtered water was added to bring the final volume to 100 mL. Thus, eight preparations were made containing roughly 50 or 100 g/L sucrose, 5 mM phosphate buffer, and roughly 0, 1, 2.5, or 10 g/L P2 primer. A 0.5-mL aliquot was withdrawn from each preparation for HPLC analysis prior to adding the glucosyltransferase enzyme (t=0). Each reaction was initiated by adding 0.226 mL of glucosyltransferase enzyme stock (for final enzyme concentration of 100 U/L). The reactions were carried out at 30° C.

At 1, 2, 3, 5 and 24 hours after commencing the reactions, 1 mL of each reaction was withdrawn to a micro-centrifuge tube. Samples were heated to 80° C. for ˜10 minutes in a dry heat block to deactivate the glucosyltransferase to stop the reaction. Deactivated samples were then spun in a table centrifuge for 10 minutes at 12,000 RPM to pellet the water-insoluble polymer products. The supernatant was withdrawn and filtered through a 0.45-μm WHATMAN syringeless filter for HPLC analysis. After 24 hours, all the reactions were placed into an 80° C. water bath for ˜30 minutes to deactivate the glucosyltransferase to stop the reaction. Polymer wet cakes were generated from each terminated reaction as described in Example 1. HPLC analysis of soluble sugars and analysis of insoluble polymer samples by NMR and SEC were carried out as described in Example 1 and the General Methods. The results of these analyses are provided in Tables 4 and 5.

TABLE 4
Structural Analysis of Alpha-1,3-Glucan Produced in
Glucosyltransferase Reactions (24-hour) Containing P2 Primer

| | | Alpha-1,3-Glucan Product Profile | | | | | | |
|---|---|---|---|---|---|---|---|---|
| Reaction | | | | | Linkages | | | |
| Sucrose | Primer | | | % | (% of total residues) | | | |
| (g/L) | (g/L) | DPw | PDI | primer | 1,3 | 1,2,6 | 1,2 | 1,6 |
| 53 | 0.0 | 841.0 | 1.7 | 0.0 | 100.0 | 0.0 | 0.0 | 0.0 |
| | 1.2 | 3092.5 | 1.9 | 4.1 | 95.9 | 0.2 | 0.3 | 3.6 |
| | 2.6 | 2615.3 | 1.7 | 6.9 | 93.1 | 0.4 | 0.5 | 6.1 |
| | 9.8 | 2049.2 | 1.9 | 10.5 | 89.5 | 0.6 | 0.6 | 9.3 |
| 106 | 0.0 | 850 | 1.6 | 0.0 | 100.0 | 0.0 | 0.0 | 0.0 |
| | 1.1 | 2459.3 | 2.8 | 2.1 | 97.9 | 0.1 | 0.1 | 1.9 |
| | 2.6 | 2118.7 | 2.3 | 4.2 | 95.8 | 0.2 | 0.3 | 3.7 |
| | 10.0 | 1769.8 | 2.0 | 8.2 | 91.8 | 0.5 | 0.5 | 7.2 |

TABLE 5
Profiles of Glucosyltransferase Reactions
(24-hour) Containing P2 Primer

| | | Alpha-1,3- | | Glucan/ | Glucan/ |
|---|---|---|---|---|---|
| | | Glucan | Sucrose | Primer | Primer |
| Sucrose | Primer | Yield | Rate | 3 hour | 24 hour |
| (g/L) | (g/L) | (Glucosyl) | (g/L/h) | (M ratio)^{a} | (M ratio)^{a} |
| 53 | 0.0 | 81% | 7.7 | NA | NA |
| | 1.2 | 80% | 9.9 | 1.9 | 2.3 |
| | 2.6 | 84% | 11.9 | 1.5 | 1.4 |
| | 9.8 | 88% | 14.2 | 1.3 | 1.2 |
| 106 | 0.0 | 81% | 10.0 | NA | NA |
| | 1.0 | 81% | 13.2 | 2.3 | 4.3 |
| | 2.6 | 83% | 16.0 | 1.6 | 2.3 |
| | 10.0 | 87% | 17.9 | 1.5 | 1.6 |

^{a}Glucan/primer molar ratio was calculated using DPw (estimated) of glucan arm (841) for 53 g/L sucrose reactions, 850 for 106 g/L sucrose reactions) and DPw of primer (90).

As indicated in Tables 4 and 5, significantly improved priming was observed compared to reactions in Example 1 (Tables 2 and 3). In general, and as a trend, the P2 priming reactions exhibited higher reaction rates, higher yield, and likely most importantly, a higher percentage of primer incorporation. The glucan-to-primer ratios were also better at both the 3- and 24-hour time points, implying that there were more consistent priming reactions. Increases in alpha-1,3-glucan molecular weight were also observed for all the P2-primed reactions (especially at 1 g/L), and the values seemed higher than those from corresponding reactions using P1 primer. Similar to reactions using primer P1, the effect of priming with P2 on alpha-1,3-glucan DPw and primer consumption was less in reactions with 100 g/L sucrose than those with 50 g/L sucrose.

Thus, it is fair to conclude that the P2 primer, which had a lower branching frequency (9.2% alpha-1,2 branches compared to 18.6% in P1 primer), but the same dextran backbone as the P1 primer, allowed for more efficient alpha-1,3-glucan synthesis compared to the P1 primer. This conclusion suggests that the addition of alpha-1,2 branches to the dextran backbone of a primer downregulates its ability to prime glucan synthesis in a glucosyltransferase reaction. This observation was surprising because it had been expected that the addition of more non-reducing ends (more free hydroxyl groups) to a dextran primer by a branching enzyme would have allowed for better priming activity, and therefore better alpha-1,3-glucan synthesis. Alpha-1,2 branching technology can therefore be used as an input for regulating/controlling glucan synthesis in primed glucosyltransferase reactions.

### EXAMPLE 3

### Producing Alpha-1,3-Glucan in Glucosyltransferase Reactions Containing Dextran with 9.2% Alpha-1,2 Branches (2-Liter Scale)

This Example describes scaling up the P2-primed reactions described in Example 2, which were done in shake flasks at a 100-mL scale, to 2-L reactor scale. Other than the reaction volumes, similar reaction conditions (as described in Example 2) were used in this Example to synthesize graft copolymers comprising a dextran backbone and alpha-1,3-glucan arms. Thus, in brief, this Example describes additional alpha-1,3-glucan synthesis reactions primed with dextran previously modified to have 9.2% alpha-1,2 branches.

Two glucosyltransferase reactions were prepared. Each was made by dissolving 200 grams of sucrose in ˜1 L of water, with addition of 10 mL of 1 M sodium phosphate buffer (pH 5.5) and either 9.61 or 48.05 mL of a 208 g/L P2 primer stock. The final volumes of both preparations were adjusted to 2 L using a volumetric flask. The final primer concentrations were about 1 and 5 g/L, respectively. Each preparation was filtered through a 0.22-micron filter, and then charged into a 2-L glass-jacketed reactor fitted with overhead stirring. A 0.5-mL aliquot was withdrawn from each preparation for HPLC analysis prior to adding the glucosyltransferase enzyme (t=0). Each reaction was initiated by adding 4.535 mL of glucosyltransferase enzyme stock (for final enzyme concentration of 100 U/L) (same enzyme as used in above Examples). The reactions were carried out at 30° C.

At 1, 2, 3 and 24 hours after commencing the reactions, 1 mL of each reaction was withdrawn to a micro-centrifuge tube. Samples were heated to 80° C. for ˜10 minutes in a dry heat block to deactivate the glucosyltransferase to stop the reaction. Deactivated samples were then spun in a table centrifuge for 10 minutes at 12,000 RPM to pellet the water-insoluble polymer products. The supernatant was withdrawn and filtered through a 0.45-μm WHATMAN syringeless filter for HPLC analysis. After 24 hours, both reactions were placed into an 80° C. water bath for ˜30 minutes to deactivate the glucosyltransferase to stop the reaction. Both terminated reactions were loaded on a ˜40-μm CHEMRUS disposable fritted filter, and fully suction-filtered. Retained material was then washed with ˜3×1-L de-ionized (DI) water to remove any residual soluble components, leaving behind a clean glucan polymer wet cake. Approximately 300-500 mg of each wet cake was reserved for SEC analysis. Each remainder was dried in a vacuum oven at 40° C., −40 cmHg. HPLC analysis of soluble sugars and analysis of insoluble polymer samples by NMR and SEC were carried out as described in Example 1 and the General Methods. The results of these analyses are provided in Tables 6 and 7.

TABLE 6
Structural Analysis of Alpha-1,3-Glucan Produced in
Glucosyltransferase Reactions (24-hour, 2-L) Containing P2 Primer

| | | Alpha-1,3-Glucan Product Profile | | | | | | |
|---|---|---|---|---|---|---|---|---|
| Reaction | | | | | Linkages | | | |
| Sucrose | Primer | | | % | (% of total residues) | | | |
| (g/L) | (g/L) | DPw | PDI | primer | 1,3 | 1,2,6 | 1,2 | 1,6 |
| 104 | 1.1 | 2396.2 | 2.6 | 1.9 | 98.1 | 0.1 | 0.1 | 1.7 |
| | 5.0 | 2232.9 | 2.0 | 5.7 | 94.3 | 0.4 | 0.4 | 5.0 |

TABLE 7
Profiles of Glucosyltransferase Reactions
(24-hour, 2-L) Containing P2 Primer

| | | Alpha-1,3- | | Glucan/ | Glucan/ |
|---|---|---|---|---|---|
| | | Glucan | Sucrose | Primer | Primer |
| Sucrose | Primer | Yield | Rate | 3 hour | 24 hour |
| (g/L) | (g/L) | (Glucosyl) | (g/L/h) | (M ratio)^{a} | (M ratio)^{a} |
| 104 | 1.1 | 79% | 12.9 | 2.2 | 4.6 |
| | 5.0 | 84% | 16.7 | 1.5 | 1.6 |

^{a}Glucan/primer molar ratio was calculated using DPw (estimated) of glucan arm (850) and DPw of primer (90).

By comparing the data in Tables 6 and 7 with Tables 4 and 5, respectively, it was observed that the scaled-up reactions (2-L) of this Example provided similar results (e.g., DPw of, and primer incorporation in, alpha-1,3-glucan product) with those of the reactions performed in Example 2 on a smaller scale (100-mL).

### EXAMPLE 4

### Producing Alpha-1,3-Glucan in Glucosyltransferase Reactions Containing Dextran with 9.2% Alpha-1,2 Branches (50-Liter Scale)

This Example describes scaling up the P2-primed reactions described in Example 3, which were done at 2-L scale, to 50-L scale. Other than the reaction volumes and primer purity (discussed below), similar reaction conditions (as described in Example 2) were used in this Example to synthesize graft copolymers comprising a dextran backbone and alpha-1,3-glucan arms. Thus, in brief, this Example describes additional alpha-1,3-glucan synthesis reactions primed with dextran previously modified to have 9.2% alpha-1,2 branches.

The primers employed in Examples 1-3 had been purified prior to their use in alpha-1,3-glucan synthesis reactions. However, in this Example, the P2 primer was provided in alpha-1,3-glucan synthesis reactions in the form of a crude reaction mix resulting from the enzymatic 1,2-branching of dextran (as described in Example 2).

A glucosyltransferase reaction with the following conditions was prepared: roughly 100 g/L sucrose, 5 mM sodium phosphate (pH 5.5), roughly 10 g/L P2 primer. Briefly, a 50-L glass-jacketed reactor outfitted with overhead stirring was charged with ˜20 L of DI water, after which 5 kg sucrose, 2.3 L of a 216.1 g/L crude P2 primer stock, and 250 ml of a 1 M sodium phosphate buffer (pH 5.5) were added. The solids were allowed to dissolve with stirring before the reactor was filled to 50 L by addition of DI water. A 1-mL aliquot was withdrawn (for t=0 HPLC analysis) before initiating the reaction by adding 113.38 mL of glucosyltransferase enzyme stock (for final enzyme concentration of 100 U/L) (same enzyme as used in above Examples). The reaction was carried out at 30° C.

HPLC samples were taken at 2.5 and 24 hours after starting the reaction, and were processed and analyzed as described in Example 1. After 24 hours from starting the reaction, the entire reaction was heated to ˜80° C. for ˜30 minutes to deactivate the glucosyltransferase to stop the reaction. The terminated reaction was loaded onto a cloth filter and fully vacuum-filtered. Retained material was washed with 3×˜20-L DI water to remove any residual soluble components, and then pressed overnight by suctioning on a latex rubber sheet to form a compact clean glucan polymer wet cake. A sample for SEC was taken as described in Example 1. The wet cake was then dried in a vacuum oven for ˜3 days at 60° C., −40 cmHg. A dried polymer sample was submitted for NMR analysis as described in Example 1. The results of these analyses are provided in Tables 8 and 9.

TABLE 8
Structural Analysis of Alpha-1,3-Glucan Produced in
Glucosyltransferase Reaction (24-hour, 50-L) Containing P2 Primer

| | | Alpha-1,3-Glucan Product Profile | | | | | | |
|---|---|---|---|---|---|---|---|---|
| Reaction | | | | | Linkages | | | |
| Sucrose | Primer | | | % | (% of total residues) | | | |
| (g/L) | (g/L) | DPw | PDI | primer | 1,3 | 1,2,6 | 1,2 | 1,6 |
| 104 | 9.6 | 2224.7 | 2.1 | 8.7 | 91.3 | 0.7 | 0.6 | 7.4 |

TABLE 9
Profiles of Glucosyltransferase Reaction
(24-hour, 50-L) Containing P2 Primer

| | | Alpha-1,3- | | Glucan/ | Glucan/ |
|---|---|---|---|---|---|
| | | Glucan | Sucrose | Primer | Primer |
| Sucrose | Primer | Yield | Rate | 3 hour | 24 hour |
| (g/L) | (g/L) | (Glucosyl) | (g/L/h) | (M ratio)^{a} | (M ratio)^{a} |
| 104 | 9.6 | 80% | 17.8 | 1.5 | 1.4 |

^{a}Glucan/primer molar ratio was calculated using DPw (estimated) of glucan arm (850) and DPw of primer (90).

These results indicate that further scaled-up reactions likely perform in a manner consistent with reactions run on a smaller scale.

### EXAMPLE 5

### Producing Alpha-1,3-Glucan in Glucosyltransferase Reactions Containing Dextran Lacking Alpha-1.2 Branches

This Example describes priming alpha-1,3-glucan synthesis reactions with dextran that was not previously modified to have alpha-1,2 branches (i.e., the dextran was unbranched). This Example allows comparing the use of alpha-1,2-branched dextran as primer for alpha-1,3-glucan synthesis (see results of Examples 1-4 and 6) versus using an unbranched dextran primer.

The procedure as described in Example 1 was followed to synthesize a series of four alpha-1,3-glucan polymers using an unbranched dextran as primer. The dextran used in this Example was the same one as used in Examples 1-4, but was not subjected to alpha-1,2 branching (i.e., the dextran primer in this Example has 0% alpha-1,2 branching). This linear dextran primer is referred to herein as P3 primer, and has a molecular weight of ˜14 kDa (DPw of ˜89). Thus, each of the P1-P3 primers share the same dextran backbone, but only the P1 and P2 primers have alpha-1,2 branches.

All the reactions were prepared to have roughly 100 g/L sucrose, 5 mM sodium phosphate (pH 5.5), and about 0, 1, 2.5, or 10 g/L of P3 primer. Four plastic 250-mL shake flasks were charged with 20 ml of a 500 g/L sterile-filtered sucrose solution, 5 mL of a 100 mM sterile-filtered sodium phosphate stock (pH 5.5), and 0, 1.045, 2.612, or 10.448 mL of a 95.7 g/L P3 primer stock. DI water was added to bring the final volume to 100 mL. A 0.5-mL t=0 hour HPLC aliquot was withdrawn from each preparation before adding 0.2256 mL of a glucosyltransferase enzyme stock (for final enzyme concentration of 100 U/L) (same enzyme as used in above Examples) to initiate each reaction. The reactions were carried out at 30° C.

At 1, 2, 3, 4 and 24 hours following reaction initiation, 1-mL samples were withdrawn for HPLC analysis. HPLC samples were processed and analyzed as described in Example 1. At 24 hours following reaction initiation, all reaction vessels were placed in an 80° C. water bath for ˜30 minutes to deactivate the glucosyltransferase enzyme thereby stopping the reactions. The terminated reactions were then loaded onto a 0.45-micron NALGENE RAPID FLOW disposable PES filter and fully suction-filtered. Retained material was then washed with 3×100 mL of DI water to remove any residual soluble components, leaving behind a clean glucan polymer wet cake. Each wet cake was then dried in a vacuum oven at 40° C., −40 cmHg. HPLC analysis of soluble sugars and analysis of insoluble polymer samples by NMR and SEC were carried out as described in Example 1 and the General Methods. The results of these analyses are provided in Tables 10 and 11.

TABLE 10
Structural Analysis of Alpha-1,3-Glucan Produced in
Glucosyltransferase Reactions (24-hour) Containing P3 Primer

| | | Alpha-1,3-Glucan Product Profile | | | | | | |
|---|---|---|---|---|---|---|---|---|
| Reaction | | | | | Linkages | | | |
| Sucrose | Primer | | | % | (% of total residues) | | | |
| (g/L) | (g/L) | DPw | PDI | primer | 1,3 | 1,2,6 | 1,2 | 1,6 |
| 108 | 0.0 | 919.0 | 1.7 | 0.0 | 100.0 | 0.0 | 0.0 | 0.0 |
| | 1.0 | 3429.0 | 2.7 | 2.4 | 97.6 | 0.0 | 0.0 | 2.3 |
| | 2.5 | 2940.0 | 2.0 | 4.5 | 95.5 | 0.0 | 0.0 | 4.5 |
| | 10.2 | 2000.0 | 2.0 | 8.1 | 91.9 | 0.0 | 0.0 | 8.1 |

TABLE 11
Profiles of Glucosyltransferase Reactions
(24-hour) Containing P3 Primer

| | | Alpha-1,3- | | Glucan/ | Glucan/ |
|---|---|---|---|---|---|
| | | Glucan | Sucrose | Primer | Primer |
| Sucrose | Primer | Yield | Rate | 3 hour | 24 hour |
| (g/L) | (g/L) | (Glucosyl) | (g/L/h) | (M ratio)^{a} | (M ratio)^{a} |
| 108 | 0.0 | 84% | 18.3 | NA | NA |
| | 1.0 | 80% | 13.4 | 2.4 | 4.5 |
| | 2.5 | 83% | 17.3 | 1.7 | 2.0 |
| | 10.2 | 86% | 20.5 | 2.0 | 1.4 |

^{a}Glucan/primer molar ratio was calculated using DPw (estimated) of glucan arm (919) and DPw of primer (89).

As shown in Tables 10-11, a series of four alpha-1,3-glucan polymers was synthesized in glucosyltransferase reactions comprising an unbranched dextran primer. Some interesting differences were observed by comparing the results of this Example (using unbranched dextran primer P3) with those of Example 2 (using 9.2% alpha-1,2-branched primer P2), both of which Examples utilized the same reaction conditions except for different primers. For example, there appeared to be a more substantial increase in alpha-1,3-glucan yield when ˜2.5 g/L or ˜10 g/L of P2 primer was used (Table 5) as opposed to when ˜2.5 g/L or ˜10 g/L of P3 primer was used (Table 11). As another example, the molecular weights of alpha-1,3-glucan products produced using P2 primer (and P1 primer) were generally less than the molecular weights observed when using P3 primer. Alpha-1,2-branched dextran primers could therefore be useful for down-regulating alpha-1,3-glucan product molecular weight.

### EXAMPLE 6

### Producing Alpha-1,3-Glucan in Glucosyltransferase Reactions Containing Dextran with ˜29.2% Alpha-1,2 Branches

This Example, in addition to Examples 1-4, describes how the efficacy of dextran priming of alpha-1,3-glucan synthesis can be controlled by varying the alpha-1,2 branching frequency on the dextran primer. In particular, this Example shows that a dextran primer with about 29.2% alpha-1,2-branches failed to prime alpha-1,3-glucan synthesis in a glucosyltransferase reaction. Alpha-1,3-glucan homopolymer was produced, whereas very little or no alpha-1,3-glucan was synthesized from dextran primer.

The procedure as described in Example 1 was followed to synthesize a series of four alpha-1,3-glucan graft copolymers using a dextran primer having ˜29.2% alpha-1,2 branches. This dextran primer is referred to herein as P4 primer, and has a molecular weight of ˜22 kDa (DPw of ˜139). The P4 primer shares the same dextran backbone as primers P1-P3.

All the reactions were prepared to have roughly 100 g/L sucrose, 5 mM sodium phosphate (pH 5.5), and about 0, 1, 5, or 10 g/L of P4 primer. Four plastic 250-mL shake flasks were charged with 20 mL of a 500 g/L sterile-filtered sucrose solution, 5 mL of a 100 mM sterile-filtered sodium phosphate stock (pH 5.5), and 0, 0.504, 2.520 or 5.040 mL of a 198.4 g/L P4 primer stock. DI water was added to bring the final volume to 100 mL. A 0.5-mL t=0 hour HPLC aliquot was withdrawn from each preparation before adding 0.2256 mL of a glucosyltransferase enzyme stock (for final enzyme concentration of 100 U/L) (same enzyme as used in above Examples) to initiate each reaction. The reactions were carried out at 30° C.

At 1, 2, 3, 4 and 24 hours following reaction initiation, 1-mL samples were withdrawn for HPLC analysis. HPLC samples were processed and analyzed as described in Example 1. At 24 hours following reaction initiation, all reaction vessels were placed in an 80° C. water bath for ˜30 minutes to deactivate the glucosyltransferase enzyme thereby stopping the reactions. A polymer wet cake was generated as described in Example 4. HPLC analysis of soluble sugars and analysis of insoluble polymer samples by NMR and SEC were carried out as described in Example 1 and the General Methods. The results of these analyses are provided in Tables 12 and 13.

TABLE 12
Structural Analysis of Alpha-1,3-Glucan Produced in
Glucosyltransferase Reactions (24-hour) Containing P4 Primer

| | | Alpha-1,3-Glucan Product Profile | | | | | | |
|---|---|---|---|---|---|---|---|---|
| Reaction | | | | | Linkages | | | |
| Sucrose | Primer | | | % | (% of total residues) | | | |
| (g/L) | (g/L) | DPw | PDI | primer | 1,3 | 1,2,6 | 1,2 | 1,6 |
| 109 | 0.0 | 919.0 | 1.7 | 0.0 | 100.0 | 0.0 | 0.0 | 0.0 |
| | 0.9 | 1547.0 | 2.1 | 0.0 | 100.0 | 0.0 | 0.0 | 0.0 |
| | 5.0 | 1494.0 | 2.0 | 0.0 | 100.0 | 0.0 | 0.0 | 0.0 |
| | 9.9 | 1457.0 | 2.2 | 0.0 | 100.0 | 0.0 | 0.0 | 0.0 |

TABLE 13
Profiles of Glucosyltransferase Reactions
(24-hour) Containing P4 Primer

| | | Alpha-1,3- | | Glucan/ | Glucan/ |
|---|---|---|---|---|---|
| | | Glucan | Sucrose | Primer | Primer |
| Sucrose | Primer | Yield | Rate | 3 hour | 24 hour |
| (g/L) | (g/L) | (Glucosyl) | (g/L/h) | (M ratio)^{a} | (M ratio)^{a} |
| 109 | 0.0 | 84% | 18.3 | NA | NA |
| | 0.9 | 81% | 16.1 | 13.9 | 27.4 |
| | 5.0 | 83% | 18.0 | 14.5 | 35.8 |
| | 9.9 | 83% | 20.8 | 9.7 | 57.8 |

^{a}Glucan/primer molar ratio was calculated using DPw (estimated) of glucan arm (919) and DPw of primer (139).

As shown in Tables 12-13, a series of four alpha-1,3-glucan polymers was synthesized in glucosyltransferase reactions comprising a ˜29.2% alpha-1,2-branched dextran primer. Based on SEC, NMR and HPLC results, minimal P4 primer was incorporated into the final insoluble alpha-1,3-glucan products. The linkage profile of the glucan products in reactions containing the P4 primer was nearly identical to that of the product of the reaction not containing the P4 primer. Only a tiny amount of alpha-1,6 linkages was observed by NMR, but were below quantifiable levels, for glucan products of P4-containing reactions. No significant changes in glucan product DPw were observed by SEC, and only trace amounts of primer were consumed based on HPLC results, for P4-containing reactions. Thus, it appears that nearly all of the insoluble glucan produced in P4-containing reactions was alpha-1,3-glucan homopolymer.

As noted in Example 5, the molecular weights of alpha-1,3-glucan products produced using P2 primer (9.2% alpha-1,2 branches) or P1 primer (18.6% alpha-1,2 branches) were generally less than the product molecular weights observed when using P3 primer (no alpha-1,2 branches). It is noted in this Example that the same phenomenon also occurred when using the P4 primer (29.2% alpha-1,2 branches). Further, by comparing Tables 2, 4, 10 and 12, it is apparent that, as the percent of primer alpha-1,2 branching increases, the molecular weight of alpha-1,3-glucan product (of a reaction comprising the primer) decreases. This is further evidence that alpha-1,2-branched dextran primers could be useful for down-regulating alpha-1,3-glucan product molecular weight in glucosyltransferase reactions.

### EXAMPLE 7

### Preparation and Use of Film Comprising Dextran-Alpha-1,3-Glucan Graft Copolymer

This Example describes producing film comprising dextran-alpha-1,3-glucan graft copolymer as produced in alpha-1,3-glucan synthesis reactions primed with P2 primer (dextran with 9.2% alpha-1,2 branches). This film was compared to film comprising insoluble alpha-1,3-glucan homopolymer as produced in glucosyltransferase reactions lacking dextran primer. Film comprising the dextran-alpha-1,3-glucan graft copolymer exhibited enhanced performance characteristics compared to film comprising alpha-1,3-glucan homopolymer.

### Materials

Sodium hydroxide was obtained from Fisher Scientific (Fair Lawn, NJ). Sulfuric acid and sodium sulfate were obtained from EMD Millipore (Darmstadt, Germany). Dextran-alpha-1,3-glucan graft copolymer preparations were as described in Example 3 (Tables 6-7, product of alpha-1,3-glucan synthesis reaction comprising 5.0 g/L of P2 primer and 104 g/L of sucrose, termed herein as “F-104-5”), Example 3 (Tables 6-7, product of alpha-1,3-glucan synthesis reaction comprising 1.1 g/L of P2 primer and 104 g/L of sucrose, termed herein as “F-104-1”), and Example 4 (Tables 8-9, product of alpha-1,3-glucan synthesis reaction comprising 9.6 g/L of P2 primer and 104 g/L of sucrose, termed herein as “F-104-10”). Alpha-1,3-glucan homopolymer (DPw ˜800) was prepared in an unprimed glucosyltransferase reaction comprising about 100 g/L sucrose and the non-native glucosyltransferase used in the above Examples.

### Solution and Film Preparation

Solutions of each alpha-1,3-glucan polymer were prepared using an IKA overhead stirrer with a PTFE oval paddle blade. Slurries of polymer and water (37.5 g total mass) were mixed by hand and then stir-added to 22.5 g of a 10 wt % solution of NaOH, after which the solutions were stirred for 3 hours. An appropriate amount of polymer was used to achieve a particular desired final polymer concentration (10-12 wt %, see Table 14). Each polymer's moisture content was measured and accounted for during solution preparation. Each solution was transferred to a FALCON tube and centrifuged to de-aerate the solution.

For film preparation, solutions were poured onto individuals glass plates with 5-mil tape on the sides (to control film thickness) and casted using a glass rod. Films were then coagulated in an acid bath (14 wt % sulfuric acid, 25 wt % sodium sulfate, 61 wt % DI water) for 3 minutes. Films were then washed in DI water to a neutral pH, transferred to a 5 wt % glycerol bath for 3 minutes, and then placed in an oven at 80° C. for 15 minutes.

### Film Test Methods

Film thickness was determined using a FEDERAL 22P-10 micrometer. Tensile properties were measured on an INSTRON 5500R Model 1122 using 1-inch grips and a 2-inch gauge length, in accordance with ASTM D882-09 (“Standard Test Method for Tensile Properties of Thin Plastic Sheeting”), which is incorporated herein by reference. Films were cut into 3-inch dog bones for tensile strength measurements, which are represented in Table 14 below as an average of 4-5 breaks.

TABLE 14
Characteristics of Films Comprising Alpha-1,3-Glucan Polymers

| | Film Comprising: | | | | | |
|---|---|---|---|---|---|---|
| | Alpha-1,3- | | | | | |
| | Glucan | | | | | |
| | Homopolymer | | | | | |
| Property | Only | F-104-5^{a} | F104-10^{b} | | | F104-1^{c} |
| Solution | 12 | 10 | 10 | 12 | 12 | 10 |
| solids (wt %) | | | | | | |
| Thickness | 0.89 | 0.92 | 0.95 | 1.36 | 0.98 | 0.89 |
| (mil) | | | | | | |
| ± | 0.064 | 0.034 | 0.051 | 0.055 | 0.049 | 0.056 |
| Width (in.) | 0.5 | 0.5 | 0.5 | 0.5 | 0.5 | 0.5 |
| Initial | 543.6 | 417.86 | 344.35 | 614.17 | 516.66 | 486.21 |
| Modulus (MPa) | | | | | | |
| ± | 45.385 | 52.545 | 63.135 | 122.286 | 123.832 | 74.08 |
| Stress @ | 14.619 | 16.144 | 13.087 | 19.096 | 17.564 | 14.747 |
| Max (MPa) | | | | | | |
| ± | 1.542 | 2.141 | 0.565 | 1.974 | 1.868 | 2.12 |
| % Elongation | 17.25 | 33.84 | 33.48 | 20.02 | 21.99 | 26.99 |
| at Break | | | | | | |
| ± | 4.44 | 7.997 | 4.342 | 1.969 | 6.562 | 7.401 |
| WTM ^{d} | 0.12 | 0.265 | 0.224 | 0.295 | 0.212 | 0.196 |
| (in-lb./in) | | | | | | |
| ± | 0.043 | 0.092 | 0.042 | 0.027 | 0.055 | 0.073 |
| Toughness | 269.066 | 579.656 | 469.127 | 435.411 | 429.626 | 441.463 |
| (in-lb./in³) | | | | | | |
| ± | 86.285 | 203.173 | 73.130 | 58.528 | 91.178 | 160.838 |

^{a}Alpha-1,3-glucan product of glucan synthesis reaction comprising 5.0 g/L of P2 primer and 104 g/L of sucrose (Tables 6-7, Example 3).
^{b}Alpha-1,3-glucan product of glucan synthesis reaction comprising 1.1 g/L of P2 primer and 104 g/L of sucrose (Tables 6-7, Example 3).
^{c}Alpha-1,3-glucan product of glucan synthesis reaction comprising 9.6 g/L of P2 primer and 104 g/L of sucrose (Tables 8-9, Example 4).
^{d} WTM, “work-to-max”. WTM is dependent on the maximum load measurement, indicating the work necessary to break the film.

As shown in Table 14, film comprising alpha-1,3-glucan synthesized in a glucosyltransferase reaction primed with alpha-1,2-branched (9.2%) dextran exhibited, for example, an improvement in elongation (e.g., % elongation at break) and maintained strength (Stress @ Max), as compared to film comprising alpha-1,3-glucan homopolymer. Also, toughness and WTM were improved. Thus, film comprising a dextran-alpha-1,3-glucan graft copolymer can exhibit enhanced performance characteristics compared to film comprising alpha-1,3-glucan homopolymer.

### EXAMPLE 8

### Reactions Containing Dextran with 18.6% Alpha-1,2 Branches and Glucosyltransferase that Produces Low Molecular Weight Alpha-1,3-Glucan

This Example describes synthesis of alpha-1,3-glucan in glucosyltransferase reactions containing a dextran primer that was previously modified to have 18.6% alpha-1,2 branches. The glucosyltransferase enzyme used in these reactions was modified to produce alpha-1,3-glucan of lower molecular weight compared to glucan product made by the enzyme's unmodified counterpart. Graft copolymers were synthesized comprising a dextran backbone and alpha-1,3-glucan arms.

Alpha-1,2-branched dextran was produced to prime glucosyltransferase reactions for alpha-1,3-glucan synthesis. Preparation of this primer was performed as described in Example 1 (above) for the P1 primer The resulting dextran, which had a DPw of about 104 (˜17 kDa) and 18.6% alpha-1,2-branches, is referred to herein as P5 primer.

P5 primer was then included in alpha-1,3-glucan synthesis reactions comprising a glucosyltransferase to produce graft copolymers comprising a dextran backbone and alpha-1,3-glucan arms. The glucosyltransferase used in these reactions, which produces alpha-1,3-glucan with about 100% alpha-1,3 linkages, was a Streptococcus salivarius glucosyltransferase modified in its catalytic domain such that the enzyme could produce alpha-glucan of lower molecular weight, as compared to the molecular weight of alpha-glucan produced by the enzyme's unmodified counterpart. The General Methods section describes assaying this non-native glucosyltransferase (Table D). Enzyme preparation in this Example was conducted as follows. Briefly, KOBW-3a cells heterologously expressing the glucosyltransferase were grown in LB medium containing 100 ng/ml ampicillin and 0.025% L-arabinose at 30° C. overnight. Cells were pelleted by centrifugation and then lysed with a 10% volume of BugBuster® Protein Extraction Reagent (EMD Millipore) at room temperature for at least 1 hour; cell debris was removed by centrifugation, after which clear cell lysate was collected for testing.

Thirteen separate 4.0-mL alpha-1,3-glucan synthesis reactions were performed in glass 50-mL indented flasks. Each reaction comprised water, sucrose (roughly 20, 50, or 200 g/L), phosphate buffer (5 mM, pH 5.7), optionally P5 primer (roughly 0.2, 1.0, or 5.0 g/L), and a glucosyltransferase (above). The reactions were prepared by mixing an appropriate amount of P5 primer (using 50 g/L stock solution), 0.2 mL of cell lysate (containing the glucosyltransferase) and corresponding buffer. Each of these preparations and a sucrose stock solution (400 g/L) was warmed at 35° C. for at least 30 minutes. An appropriate volume of sucrose solution was then added to each preparation to initiate alpha-1,3-glucan synthesis. The reactions were carried out at 35° C. with shaking (10 rpm) for about 60 hours. Following this incubation, the entire contents of the reactions were individually transferred to 15-mL centrifuge tubes, which were placed in an 85° C. oven for about 30 minutes to deactivate the glucosyltransferase thereby terminating the reactions. Approximately 300-500 mg of wet cake produced in each reaction was analyzed by SEC to determine the molecular weight and DP of the insoluble polymer products. Table 15 provides the results of these analyses.

TABLE 15
Analysis of Alpha-1,3-Glucan Produced in Glucosyltransferase
Reactions Containing P5 Primer

| Reaction | | | | | | |
|---|---|---|---|---|---|---|
| Primer^{a} | Sucrose | Alpha-1,3-Glucan Product Profile | | | | |
| (g/L) | (g/L) | Polymer Type | DPw | Mw | Mp | Mw/Mn |
| 0.0 | 20 | non-grafted | 58 | 9439 | 9948 | 1.22 |
| 0.2 | | grafted | 625 | 101191 | 97726 | 1.33 |
| 1.0 | | grafted | 402 | 65163 | 62668 | 1.32 |
| 5.0 | | grafted | 245 | 39628 | 35874 | 1.21 |
| 0.0 | 50 | non-grafted | 52 | 8363 | 8117 | 1.25 |
| 0.2 | | grafted | 680 | 110189 | 95028 | 1.36 |
| 1.0 | | grafted | 425 | 68878 | 68877 | 1.28 |
| 5.0 | | grafted | 251 | 40718 | 36926 | 1.22 |
| 0.0 | 200 | non-grafted | 41 | 6690 | 5945 | 1.22 |
| 0.2 | | grafted | 517 | 83679 | 88410 | 1.3 |
| 1.0 | | grafted | 374 | 60530 | 59285 | 1.24 |
| 5.0 | | grafted | 236 | 38264 | 35119 | 1.2 |

^{a}Primer P5 is DPw 104 (Mw = 16869 Da) (Mw/Mn = 1.48).

The size of individual alpha-1,3-glucan arms grafted onto primer in the glucan product of each primer-containing reaction is contemplated to be very similar to the size of alpha-1,3-glucan produced in control reactions that did not contain added primer; the control reaction products comprised alpha-1,3-glucan homopolymer. That said, it seems notable that, as the concentration of primer was increased in each set of reaction series (20, 50, or 200 g/L sucrose), graft copolymer product DPw decreased significantly.

Glucan-to-primer molar ratios (M ratios) were calculated based on the respective control DPw value (homopolymer DPw) under each tested condition and the DPw of the P5 primer (˜104). The data from these analyses are provided in Table 16.

TABLE 16
Glucan-to-Primer Molar Ratio of Alpha-1,3-Glucan Graft Copolymer
Produced in Glucosyltransferase Reactions Containing P5 Primer

| | | |
|---|---|---|
| Primer | Sucrose | Alpha-1,3-Glucan Product Profile |

| (g/L) | (g/L) | DPw | M Ratio |
|---|---|---|---|
| 0.2 | 20 | 625 | 9 |
| 1.0 | | 402 | 5 |
| 5.0 | | 245 | 2 |
| 0.2 | 50 | 680 | 11 |
| 1.0 | | 425 | 6 |
| 5.0 | | 251 | 3 |
| 0.2 | 200 | 517 | 10 |
| 1.0 | | 374 | 7 |
| 5.0 | | 236 | 3 |

Thus, by providing an alpha-1,2-branched dextran primer to alpha-1,3-glucan synthesis reactions, graft copolymers were synthesized comprising a dextran backbone and alpha-1,3-glucan arms. Primer was demonstrably consumed and incorporated into graft copolymer products.

### EXAMPLE 9

### Reactions Containing Dextran with 9.7% Alpha-1,2 Branches and Glucosyltransferase that Produces Low Molecular Weight Alpha-1,3-Glucan

This Example describes synthesis of alpha-1,3-glucan in glucosyltransferase reactions containing a dextran primer that was previously modified to have 9.7% alpha-1,2 branches. The glucosyltransferase enzyme used in these reactions was modified to produce alpha-1,3-glucan of lower molecular weight compared to glucan product made by the enzyme's unmodified counterpart. Graft copolymers were synthesized comprising a dextran backbone and alpha-1,3-glucan arms.

Alpha-1,2-branched dextran was produced to prime glucosyltransferase reactions for alpha-1,3-glucan synthesis. Preparation of this primer was performed essentially as described in International Patent. Appl. Publ. No. WO2017/091533, which is incorporated herein by reference. Briefly, linear dextran (i.e., polysaccharide with 100% alpha-1,6 glycosidic linkages) of molecular weight ˜40 kDa (DPw of ˜250) was produced using glucosyltransferase GTF6831 (SEQ ID NO:68). This dextran was then modified to have 9.7% alpha-1,2-branches using alpha-1,2-branching enzyme GTFJ18T1 (SEQ ID NO:70). The resulting alpha-1,2-branched dextran, which had a DPw of about 271 (˜44 kDa), is referred to herein as P6 primer.

P6 primer was then included in alpha-1,3-glucan synthesis reactions comprising a glucosyltransferase to produce graft copolymers comprising a dextran backbone and alpha-1,3-glucan arms. The glucosyltransferase used in these reactions was the same as used and prepared in Example 8.

Thirteen separate 4.0-mL alpha-1,3-glucan synthesis reactions were performed in glass 50-mL indented flasks. Each reaction comprised water, sucrose (roughly 20, 50, or 200 g/L), phosphate buffer (5 mM, pH 5.7), optionally P6 primer (roughly 0.2, 1.0, or 5.0 g/L), and a glucosyltransferase (above). The reactions were prepared by mixing an appropriate amount of P6 primer (using 50 g/L stock solution), 0.2 mL of cell lysate (containing the glucosyltransferase) and corresponding buffer. Each of these preparations and a sucrose stock solution (400 g/L) was warmed at 35° C. for at least 30 minutes. An appropriate volume of sucrose solution was then added to each preparation to initiate alpha-1,3-glucan synthesis. The reactions were carried out at 35° C. with shaking (10 rpm) for about 60 hours. Following this incubation, the entire contents of the reactions were individually transferred to 15-mL centrifuge tubes, which were placed in an 85° C. oven for about 30 minutes to deactivate the glucosyltransferase thereby terminating the reactions. Approximately 300-500 mg of wet cake produced in each reaction was analyzed by SEC to determine the molecular weight and DP of the insoluble polymer products. Table 17 provides the results of these analyses.

TABLE 17
Analysis of Alpha-1,3-Glucan Produced in Glucosyltransferase
Reactions Containing P6 Primer

| Reaction | | | | | | |
|---|---|---|---|---|---|---|
| Primer^{a} | Sucrose | Alpha-1,3-Glucan Product Profile | | | | |
| (g/L) | (g/L) | Polymer Type | DPw | Mw | Mp | Mw/Mn |
| 0.0 | 20 | non-grafted | 58 | 9439 | 9948 | 1.22 |
| 0.2 | | grafted | 1224 | 198295 | 222340 | 1.55 |
| 1.0 | | grafted | 1023 | 167308 | 177954 | 1.7 |
| 5.0 | | grafted | 769 | 124646 | 95437 | 1.6 |
| 0.0 | 50 | non-grafted | 52 | 8363 | 8117 | 1.25 |
| 0.2 | | grafted | 1211 | 196134 | 224349 | 1.59 |
| 1.0 | | grafted | 1085 | 175800 | 187403 | 1.58 |
| 5.0 | | grafted | 768 | 124454 | 112146 | 1.64 |
| 0.0 | 200 | non-grafted | 41 | 6690 | 5945 | 1.22 |
| 0.2 | | grafted | 993 | 160848 | 206992 | 1.58 |
| 1.0 | | grafted | 1060 | 171788 | 184475 | 1.45 |
| 5.0 | | grafted | 749 | 121391 | 114253 | 1.61 |

^{a}Primer P6 is DPw 271 (Mw = 43958 Da) (Mw/Mn = 1.19).

The size of individual alpha-1,3-glucan arms grafted onto primer in the glucan product of each primer-containing reaction is contemplated to be very similar to the size of alpha-1,3-glucan produced in control reactions that did not contain added primer; the control reaction products comprised alpha-1,3-glucan homopolymer. That said, it seems notable that generally, as the concentration of primer was increased in each set of reaction series (20, 50, or 200 g/L sucrose), graft copolymer product DPw decreased significantly.

Glucan-to-primer molar ratios (M ratios) were calculated based on the respective control DPw value (homopolymer DPw) under each tested condition and the DPw of the P6 primer (271). The data from these analyses are provided in Table 18.

TABLE 18
Glucan-to-Primer Molar Ratio of Alpha-1,3-Glucan Graft Copolymer
Produced in Glucosyltransferase Reactions Containing P5 Primer

| | | |
|---|---|---|
| Primer | Sucrose | Alpha-1,3-Glucan Product Profile |

| (g/L) | (g/L) | DPw | M Ratio |
|---|---|---|---|
| 0.2 | 20 | 1224 | 16 |
| 1.0 | | 1023 | 13 |
| 5.0 | | 769 | 9 |
| 0.2 | 50 | 1211 | 18 |
| 1.0 | | 1085 | 16 |
| 5.0 | | 768 | 10 |
| 0.2 | 200 | 993 | 18 |
| 1.0 | | 1060 | 19 |
| 5.0 | | 749 | 12 |

Thus, by providing an alpha-1,2-branched dextran primer to alpha-1,3-glucan synthesis reactions, graft copolymers were synthesized comprising a dextran backbone and alpha-1,3-glucan arms. Primer was demonstrably consumed and incorporated into graft copolymer products.

1-17. (canceled)

18. A composition comprising an ether-or ester-derivative of a graft copolymer, wherein said graft copolymer comprises:
(i) a backbone comprising a dextran, and
(ii) one or more alpha-1,3-glucan side chains comprising at least about 50% alpha-1,3 glycosidic linkages.

19. The composition of claim 18, wherein the dextran comprises 100% alpha-1,6 glycosidic linkages.

20. The composition of claim 18, wherein the weight-average degree of polymerization (DPw) of the backbone is at least about 10.

21. The composition of claim 20, wherein the DPw of the backbone is at least about 50.

22. The composition of claim 21, wherein the DPw of the backbone is at least about 500.

23. The composition of claim 18, wherein the dextran comprises a product of a dextransucrase enzyme that comprises an amino acid sequence that is at least 90% identical to SEQ ID NO:66.

24. The composition of claim 18, wherein the dextran comprises a product of a dextransucrase enzyme that comprises an amino acid sequence that is at least 90% identical to SEQ ID NO:67 or SEQ ID NO:68.

25. The composition of claim 18, wherein the dextran comprises a product of a dextransucrase enzyme that comprises an amino acid sequence that is at least 90% identical to SEQ ID NO:69.

26. The composition of claim 18, wherein the graft copolymer comprises at least two of said alpha-1,3-glucan side chains.

27. The composition of claim 26, wherein the graft copolymer comprises at least three of said alpha-1,3-glucan side chains.

28. The composition of claim 27, wherein the graft copolymer comprises at least four of said alpha-1,3-glucan side chains.

29. The composition of claim 18, wherein the alpha-1,3-glucan side chains comprise at least about 90% alpha-1,3 glycosidic linkages.

30. The composition of claim 26, wherein the alpha-1,3-glucan side chains comprise at least about 90% alpha-1,3 glycosidic linkages.

31. The composition of claim 18, wherein the DP or DPw of said one or more alpha-1,3-glucan side chains is at least about 10.

32. The composition of claim 26, wherein the DP or DPw of said at least two alpha-1,3-glucan side chains is at least about 10.

33. The composition of claim 18, wherein the composition comprises the ether-derivative of the graft copolymer.

34. The composition of claim 18, wherein the composition comprises the ester-derivative of the graft copolymer.

35. The composition of claim 18, wherein the composition is a household care product, personal care product, industrial product, pharmaceutical product, or food product.

36. The composition of claim 18, wherein the composition is a detergent composition.

37. The composition of claim 18, wherein the composition is a dishwashing detergent composition.

38. The composition of claim 37, wherein the dishwashing detergent composition is an automatic dishwashing detergent composition.

39. The composition of claim 18, wherein the composition is a fabric care composition.
